SEQUENCE LISTING (50 sequences)

SEQ ID NO: 1 - protein, 166 aa, Homo sapiens
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 2 - protein, 165 aa, Homo sapiens
SYNLLGFLQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 3 - protein, 17 aa, Homo sapiens
HYLKAKEYSHCAWTIVR

SEQ ID NO: 4 - protein, 20 aa, Artificial
  Synthetic
IDKISDVSTIVPYIGPALNI

SEQ ID NO: 5 - protein, 21 aa, Artificial
  Synthetic
AQSIALSSLMVAQAIPLVGEL

SEQ ID NO: 6 - protein, 11 aa, Artificial
  Synthetic
TVFYNIPPMPL

SEQ ID NO: 7 - protein, 22 aa, Artificial
  Synthetic
FNNFTVSFWLRVPKVSASHLET

SEQ ID NO: 8 - protein, 19 aa, Artificial
  Synthetic
QYIKANSKFIGITELKKLE

SEQ ID NO: 9 - protein, 17 aa, Artificial
  Synthetic
MQYIKANSKFIGITELK

SEQ ID NO: 10 - protein, 15 aa, Artificial
  Synthetic
DKGEIRRIWRGANNG

SEQ ID NO: 11 - protein, 19 aa, Artificial
  Synthetic
TRSAYERMCNILKGKFQTA

SEQ ID NO: 12 - protein, 22 aa, Artificial
  Synthetic
SLVGIDPFKLLQNSQVYSLIRP

SEQ ID NO: 13 - protein, 22 aa, Artificial
  Synthetic
LRVLSFIRGTKVSPRGKLSTRG

SEQ ID NO: 14 - protein, 17 aa, Artificial
  Synthetic
MSYNLLGFLQRSSNFQC

SEQ ID NO: 15 - protein, 17 aa, Artificial
  Synthetic
LGFLQRSSNFQCQKLLW

SEQ ID NO: 16 - protein, 17 aa, Artificial
  Synthetic
RSSNFQCQKLLWQLNGR

SEQ ID NO: 17 - protein, 17 aa, Artificial
  Synthetic
FQCQKLLWQLNGRLEYC

SEQ ID NO: 18 - protein, 17 aa, Artificial
  Synthetic
LWQLNGRLEYCLKDRMN

SEQ ID NO: 19 - protein, 17 aa, Artificial
  Synthetic
GRLEYCLKDRMNFDIPE

SEQ ID NO: 20 - protein, 17 aa, Artificial
  Synthetic
CLKDRMNFDIPEEIKQL

SEQ ID NO: 21 - protein, 17 aa, Artificial
  Synthetic
MNFDIPEEIKQLQQFQK

SEQ ID NO: 22 - protein, 17 aa, Artificial
  Synthetic
PEEIKQLQQFQKEDAAL

SEQ ID NO: 23 - protein, 17 aa, Artificial
  Synthetic
KQLQQFQKEDAALTIYE

SEQ ID NO: 24 - protein, 17 aa, Artificial
  Synthetic
FQKEDAALTIYEMLQNI

SEQ ID NO: 25 - protein, 18 aa, Artificial
  Synthetic
ALTIYEMLQNIFAIFRQD

SEQ ID NO: 26 - protein, 17 aa, Artificial
  Synthetic
EMLQNIFAIFRQDSSST

SEQ ID NO: 27 - protein, 17 aa, Artificial
  Synthetic
IFAIFRQDSSSTGWNET

SEQ ID NO: 28 - protein, 17 aa, Artificial
  Synthetic
RQDSSSTGWNETIVENL

SEQ ID NO: 29 - protein, 17 aa, Artificial
  Synthetic
STGWNETIVENLLANVY

SEQ ID NO: 30 - protein, 17 aa, Artificial
  Synthetic
ETIVENLLANVYHQINH

SEQ ID NO: 31 - protein, 17 aa, Artificial
  Synthetic
NLLANVYHQINHLKTVL

SEQ ID NO: 32 - protein, 17 aa, Artificial
  Synthetic
VYHQINHLKTVLEEKLE

SEQ ID NO: 33 - protein, 17 aa, Artificial
  Synthetic
NHLKTVLEEKLEKEDFT

SEQ ID NO: 34 - protein, 17 aa, Artificial
  Synthetic
VLEEKLEKEDFTRGKLM

SEQ ID NO: 35 - protein, 17 aa, Artificial
  Synthetic
LEKEDFTRGKLMSSLHL

SEQ ID NO: 36 - protein, 17 aa, Artificial
  Synthetic
FTRGKLMSSLHLKRYYG

SEQ ID NO: 37 - protein, 17 aa, Artificial
  Synthetic
LMSSLHLKRYYGRILHY

SEQ ID NO: 38 - protein, 17 aa, Artificial
  Synthetic
HLKRYYGRILHYLKAKE

SEQ ID NO: 39 - protein, 17 aa, Artificial
  Synthetic
YGRILHYLKAKEYSHCA

SEQ ID NO: 40 - protein, 17 aa, Artificial
  Synthetic
HYLKAKEYSHCAWTIVR

SEQ ID NO: 41 - protein, 17 aa, Artificial
  Synthetic
KEYSHCAWTIVRVEILR

SEQ ID NO: 42 - protein, 17 aa, Artificial
  Synthetic
HCAWTIVRVEILRNFYF

SEQ ID NO: 43 - protein, 17 aa, Artificial
  Synthetic
VRVEILRNFYFINRLTG

SEQ ID NO: 44 - protein, 16 aa, Artificial
  Synthetic
LRNFYFINRLTGYLRN

SEQ ID NO: 45 - protein, 17 aa, Artificial
  Synthetic
SYNLLGFLQRSSNFQCQ

SEQ ID NO: 46 - protein, 17 aa, Artificial
  Synthetic
LGFLQRSSNFQSQKLLW

SEQ ID NO: 47 - protein, 15 aa, Artificial
  Synthetic
SSLHLKRYYGRILHY

SEQ ID NO: 48 - protein, 13 aa, Artificial
  Synthetic
PKYVKQNTLKLAT

SEQ ID NO: 49 - protein, 11 aa, Artificial
  Synthetic
TVFYNIPPMPL

SEQ ID NO: 50 - protein, 17 aa, Artificial
  Synthetic
ENPVVHFFANIVTPRTP

(19) United States
(12) Patent Application Publication
(10) Pub. No.: US 2006/0148009 A1
(43) Pub. Date: Jul. 6, 2006

(54) Prediction and assessment of immunogenicity

(75) Inventors: Maria D.F.S. Barbosa, San Dimas, CA (US); Arthur J. Chirino, Camarillo, CA (US)

Correspondence Address: Robin M. Silva, Esq.;Dorsey & Whitney LLP, Intellectual Property Department, Suite 1000, 555 California Street, San Francisco, CA, 94104-1513, US

(73) Assignee: Xencor, Inc.

(21) Appl. No.: 11/249,692

(22) Filed: Oct. 12, 2005

Related U.S. Application Data
(60) Provisional application No. 60/659,586, filed on Mar. 8, 2005.
(60) Provisional application No. 60/618,154, filed on Oct. 12, 2004.

(51) Int. Cl. G01N 33/567

(52) U.S. Cl. 435/7.21

(57) ABSTRACT

A system and method to predict and assess immunogenicity, especially prior to on-set of immunogenic conditions. Also disclosed are methods to identify relevant peptides associated with the formation of antibodies in patients treated with a given protein therapeutic. In various aspects, the present application is directed to methods of determining the immunological compatibility of a subject with a therapeutic agent such as a proteinaceous therapeutic agent, methods of determining vaccine efficacy by determining the immunological compatibility of a subject with a therapeutic agent, and selecting a therapeutic agent for a subject in need of treatment. Methods of designing a therapeutic agent with reduced immunogenicity for a subject and methods for designing vaccines with enhanced immunogenicity for a subject are also contemplated.

This application claims of benefit under 35 U.S.C. §119(e) to U.S. Ser. Nos. 60/618,154, filed Oct. 12, 2004 and 60/659,586, filed Mar. 8, 2005, each of which is incorporated herein by reference in its entirety.

### FIELD OF THE INVENTION

The present invention relates to methods of determining the immunological compatibility of a subject with a therapeutic agent, determining the immunological compatibility of a subject with a therapeutic agent, and methods of designing variant therapeutic agents and vaccines.

### BACKGROUND OF THE INVENTION

Immunogenicity is a complex series of responses to a substance that is perceived as foreign and may include production of neutralizing and non-neutralizing antibodies, formation of immune complexes, complement activation, mast cell activation, inflammation, hypersensitivity responses, and anaphylaxis. A particularly prevalent problem in the administration of therapeutic agents, including those that include peptides or proteins (e.g. as vaccines or drugs) is the generation of improper immunogenicity responses in the patient. Properly modulating the immunogenicity of proteins may greatly improve the safety and efficacy of protein vaccines and protein drugs. The ability to predict the immunogenicity of a protein therapeutic in a particular patient, prior to administration of the therapeutic, would be extremely useful. Furthermore, methods to predict the immunogenicity of novel engineered proteins are also desirable for the development and clinical use of designed protein therapeutics.

In the case of protein vaccines, the goal is typically to promote a robust T cell or B cell-based immune response to a pathogen, cancer, toxin, or the like. For protein therapeutics, however, unwanted immunogenicity can reduce drug efficacy and lead to dangerous side effects. Immunogenicity has been clinically observed for most protein therapeutics, including drugs with entirely human sequence content, and can be highly patient-specific and unpredictable.

To elicit an immune response, a protein vaccine or therapeutic must productively interact with several classes of immune cells, including antigen presenting cells (APCs), T cells, and B cells. Each of these classes of cells recognize distinct antigen features: APCs express MHC molecules that recognize MHC agretopes, T cells express T cell receptors (TCRs) that recognize T cell epitopes in the context of peptide-MHC complexes, and B cells express MHC molecules and B-cell receptors (BCRs) that recognize B-cell epitopes. Furthermore, uptake by APCs is promoted by binding to any of a number of receptors on the surface of APCs.

Immunogenicity may be dramatically reduced by blocking any of these recognition events. Similarly, immunogenicity may be enhanced by promoting these recognition events. Several factors can contribute to protein immunogenicity, including but not limited to the protein sequence, the route and frequency of administration, and the patient population. Accordingly, modifying these and other factors may serve to modulate protein immunogenicity. A number of examples of methods to increase or decrease immunogenicity have been disclosed.

An assay to predict protein immunogenicity in humans is needed. Currently no efficient tools are available to predict protein immunogenicity to humans during early stages of drug development. Traditional animal models that have been routinely used during protein drug development can't reliably predict protein immunogenicity to humans, due to (a) differences between the immune system of those models and humans and (b) lack of 100% homology between human therapeutic protein and non-human endogenous protein. The usual practice has been to monitor patients taking an already approved protein drug regarding the formation of antibodies.

There also exists a need for the development and discovery of proteins with improved properties, including but not limited to increased efficacy, decreased side effects, decreased immunogenicity, increased solubility, and enhanced soluble prokaryotic expression. Improved interferon therapeutics may be useful for the treatment of a variety of diseases and conditions, including autoimmune diseases, viral infections, inflammatory diseases, and cancer, among others. In addition, interferons may be used to promote the establishment of pregnancy in certain mammals.

Further, there is a need for designing therapeutic agents, such as therapeutic proteins or peptides, to be less immunogenic. A key limitation of current computational protein design algorithms is that the immunological properties of the generated sequences are not explicitly considered. As immunogenicity may significantly affect the safety and efficacy of protein therapeutics and protein vaccines, methods to evaluate the immunogenicity of designed proteins intended for use as drugs or vaccines would be useful.

There is a need for additional immunogenicity reduction methods for non-human proteins, and even proteins with fully human sequences. A need still remains for methods to identify protein sequences with desired physical, chemical, biological, and immunological properties.

There exists a need for the development and discovery of interferon proteins with improved properties, including but not limited to increased efficacy, decreased side effects, decreased immunogenicity, increased solubility, and enhanced soluble prokaryotic expression. Improved interferon therapeutics may be useful for the treatment of a variety of diseases and conditions, including autoimmune diseases, viral infections, inflammatory diseases, and cancer, among others. In addition, interferons may be used to promote the establishment of pregnancy in certain mammals.

### SUMMARY OF THE INVENTION

In one aspect, the invention is directed to a method of correlating at least one MHC allele with the immunogenicity of a therapeutic agent. At least some of the MHC alleles are expressed by each of a plurality of subjects are determined. The therapeutic agent is then administered to the plurality of subjects, and the immunogenic response of each of the plurality of subjects to the therapeutic agent is measured. At least one MHC allele in at least one of the subjects is compared with the immunogenic response of at least one subject expressing said allele to correlate at least one allele with the immunogenicity of a therapeutic agent.

In another aspect, the invention is directed to a method of determining the immunological compatibility of a subject with a therapeutic agent. At least one MHC allele expressed by the subject is determined. The MHC allele is compared to an immunological correlation between the MHC allele and the immunogenicity of the therapeutic agent to determine the immunological compatibility of the subject with the therapeutic agent.

In a further aspect, the invention is directed to a method of selecting a therapeutic agent for a subject in need of treatment. The immunological compatibility of the subject with the therapeutic agent is determined. The subject is then treated with the therapeutic agent if the subject is immunologically compatible with the therapeutic agent.

In a further aspect, the present invention is directed to a method of designing a therapeutic agent with reduced immunogenicity for a subject by determining the immunological compatibility of a subject and a therapeutic agent and designing a derivative having reduced immunogenicity to the therapeutic agent.

In certain variations of the disclosed methods, the subject is human. In other variations, the therapeutic agent includes either a protein. In some additional aspects, the protein is interferon-β. In certain aspects of the invention in which a peptide or protein containing derivative is designed, the derivative has a decreased binding affinity to the MHC allele.

### BRIEF DESCRIPTION OF THE FIGURES

FIG. 1 is a flowchart exemplifying an embodiment of the present invention utilizing interferon beta as the target protein.

FIG. 2 is a graph showing selection of donor/peptide combination for assay optimization.

FIG. 3 shows the effect of various DMSO concentrations on the Elispot T cell activation assay using PBMCs.

FIG. 4 shows the effect of various EBV peptide concentrations on the T cell activation assay using PBMCs.

FIG. 5 is a graph showing the effect of cell number on the T cell activation.

FIG. 6 is a graph showing a T cell activation Elispot assay.

FIG. 7 is a graph showing a T cell activation Elispot assay.

FIG. 8 is a graph showing a T cell activation Elispot assay.

FIG. 9 is a graph showing the dose-response for a peptide mix (NP1+NP2+NP3+NP4) in a T cell activation Elispot assay.

FIG. 10(A) is a graph showing the Dose-response for peptides in a T cell activation Elispot assay using two different peptide/donor combinations; FIG. 10(B) is a graph showing the average values for the controls.

FIG. 11(A) a graph showing the effect of cell number on a T cell activation Elispot assay with two peptide/donor combinations. FIG. 10(B) is a graph showing the average values for the controls.

FIG. 12 shows the effect of added cytokines on a T cell activation Elispot assay. FIG. 12(A) control with no cytokines added; FIG. 12(B), 1 ng/ml of each IL-7 and IL-1 5 added to the medium.

FIG. 13 is a graph showing the effect of added cytokines on a T cell activation Elispot assay.

FIG. 14 is a graph showing the effect of added cytokines on a T cell activation Elispot assay.

FIG. 15 is a graph showing the results from DELFIA assay (direct capture ELISA-DCE) probing for IFNβ-reactive IgGs in Betaseron treated patient serum samples. Donors are denoted with the last three digits of their identifier code.

FIG. 16 is the IFN-β 1a amino acid sequence (SEQ ID NO:1).

FIG. 17 is the IFN-β 1b amino acid sequence.(SEQ ID NO:2)

FIG. 18 is Peptide scan with samples from a normal donor, not in IFN-β therapy.

FIG. 19 is IFN-β epitope mapping using a T cell activation Elispot assay.

FIG. 20 is IFN-β epitope mapping using a T cell activation Elispot assay and PBMC. In FIG. 20(A) and (B) the background from the negative control without antigen was subtracted. FIG. 20(C) shows images of wells assayed in the presence of IFN-β peptide pools or controls.

FIG. 21 is IFN-β epitope mapping using a T cell activation Elispot assay and PBMC from a donor that was positive for IFN-β binding antibody assay. In FIG. 21(A) and (B) the background from the negative control without antigen was subtracted. FIG. 21(C) shows images of wells assayed in the presence of IFN-β peptide pools or controls.

FIG. 22 shows IFN-γ production by T-cells tested with an Elispot assay.

FIG. 23 shows IFN-β epitope mapping using cells from human subject 4957 in an Elispot assay.

FIG. 24 shows IFN-β epitope mapping using cells from human subject 6424 in an Elispot assay.

FIG. 25(A, B and C) shows results for an Elispot assay using PBMCs from both anti-interferon-beta antibody positive or negative subjects.

FIG. 26 shows development of a binding assay using B-cell lines with the HLA-11 type DRB1*0101.

FIG. 27 shows Bio-HA peptide binding to DRB1*0101 in the presence of anti DQ and anti DP antibodies.

FIG. 28 shows IFN-β agretopes identified with a peptide-binding assay.

FIG. 29 shows dose-dependent competition of EBV280-290 peptide binding to B cells with the interferon beta peptide HYLKAKEYSHCAWTIVR. (SEQ ID NO:3)

### DETAILED DESCRIPTION

In various aspects, the present application is directed to methods of determining the immunological compatibility of a subject with a therapeutic agent such as a proteinaceous therapeutic agent, determining the immunological compatibility of a subject with a therapeutic agent, and selecting a therapeutic agent for a subject in need of treatment. Methods of designing a therapeutic agent with reduced immunogenicity in a subject are also contemplated. Further, methods of designing a vaccine with increased immunogenicity in a subject are contemplated.

Definitions

Before describing the invention in greater detail, the following definitions are set forth to illustrate and define the meaning and scope of the terms used to describe the invention herein.

By “immunological compatibility” and grammatical equivalents is meant the degree of an immune response to a therapeutic agent by cells or subjects. The immune response can be cellular or humoral. Immunological compatibility can be measured as an increased or decreased immune response. By way of example, reduced immunological compatibility refers to an increase in neutralizing or non-neutralizing antibodies to a therapeutic agent. The level of immunological compatibility can depend on the assay. For example, in certain assays, a ratio of anti-therapeutic protein antibodies above a specific value indicates a positive result for antibodies. By way of example, an antibody ratio of 0.05, 0.06, 0.07, 0.08, 0.09, 0.10, 0.2, 0.3, 0.4, 0.5, 0.6, 0.7. 0.8, 0.9, or 0.10 within statistical error can indicate the absence of immunological compatibility with a therapeutic agent. Alternatively, immunological compatibility can be measured by an increase in antibody titer to a therapeutic agent as compared to a control.

By “immunologically compatible” and grammatical equivalents is meant that a therapeutic agent does not have increased immunogenicity to a therapeutic agent above that observed in control subjects. A subject is immunologically compatible of an immune response to a therapeutic agent is not above a specific value.

By “therapeutic agent” is meant a compound capable of having a physiological effect on a subject in response to a disease state or disorder. The “therapeutic agent” is used to treat a subject in need of treatment, such as for a disease or condition. In certain embodiments, the therapeutic agent is proteinaceous (i.e. includes a protein). In additional embodiments, the therapeutic agent is a protein (e.g. therapeutic protein).

By “immunological correlation” is meant a correlation between one or more immunological responses and a therapeutic agent. For example, under certain circumstances an immunological correlation means that a therapeutic agent has reduced immunological compatibility when a therapeutic agent is administered to a subject with a specific MHC allele, group of alleles, or allotype. Alternatively, the immunological correlation means reduced immunological compatibility in a certain percentage of subjects(e.g. 10%, 20%, 30%, 40%, 50%, or 60% of subjects).

By “immune response” and “immunological response” and grammatical equivalents is meant a response of the immune system to a molecule, include humoral or cellular immune response. Non-limiting immunological responses include production of neutralizing and non-neutralizing antibodies, formation of immune complexes, complement activation, mast cell activation, inflammation, and anaphylaxis. Immunogenicity is species-specific. In certain embodiments, immunogenicity refers to immunogenicity in humans. In alternate embodiments, immunogenicity refers to immunogenicity in rodents, (rats, mice, hamster, guinea pigs, etc.), primates, farm animals (including sheep, goats, pigs, cows, horses, etc.), and domestic animals, (including cats, dogs, rabbits, etc).

By “9-mer peptide frame” and grammatical equivalents herein is meant a linear sequence of nine amino acids. 9-mer frames may be analyzed for their propensity to bind one or more class II MHC alleles.

By “allele” and grammatical equivalents herein is meant an alternative form of a gene or protein. In the context of human class II MHC molecules, alleles include all naturally occurring sequence variants of a DRA, DRB1, DRB3/4/5, DQA1, DQB1, and DPB1 molecules. In the context of human class I MHC molecules, alleles include all naturally occurring sequence variants of HLA-A, HLA-B, and HLA-C molecules.

By “anchor residue” and grammatical equivalents herein is meant a position in an MHC agretope that is especially important for conferring MHC binding affinity or determining whether a given sequence will bind a given MHC allele. For example, the P1 position is an anchor residue for DR alleles, as the presence of a hydrophobic residue at P1 is required for DR binding.

By “antibody epitope” or “B-cell receptor epitope” and grammatical equivalents herein is meant one or more residues in a protein that are capable of being recognized by one or more antibodies. As is known in the art, antibody epitopes may comprise “conformational epitopes,” or sets of residues that are located nearby in the tertiary structure of the protein but are not adjacent in the primary sequence. By “antigenicity” and grammatical equivalents herein is meant the ability of a molecule, for example a protein, to be recognized by antibodies.

By “computational immunogenicity filter” herein is meant any of a number of computational algorithms that is capable of differentiating protein sequences on the basis of immunogenicity. Computational immunogenicity filters include scoring functions that are derived from data on binding of peptides to MHC and TCR molecules as well as data on protein-antibody interactions. In a preferred embodiment, the immunogenicity filter comprises matrix method calculations for the identification of MHC agretopes.

By “computational protein design algorithm” and grammatical equivalents herein is meant any computational method that may be used to identify variant protein sequences that are capable of folding to a desired protein structure or possessing desired functional properties. In a preferred embodiment the computational protein design algorithm is Protein Design Automation® technology. Protein Design Automation® as used herein includes

By “conservative modification” and grammatical equivalents herein is meant a modification in which the parent protein residue and the variant protein residue are substantially similar with respect to one or more properties such as hydrophobicity, charge, size, and shape.

By “hit” and grammatical equivalents herein is meant, in the context of the matrix method, that a given peptide is predicted to bind to a given class II MHC allele. In a preferred embodiment, a hit is defined to be a peptide with binding affinity among the top 5%, or 3%, or 1% of binding scores of random peptide sequences. In an alternate embodiment, a hit is defined to be a peptide with a binding affinity that exceeds some threshold, for instance a peptide that is predicted to bind an MHC allele with at least 100 μM or 10 μM or 1 μM affinity.

By “immunogenic sequences” herein is meant sequences that promote immunogenicity, including but not limited to antigen processing cleavage sites, class I MHC agretopes, class II MHC agretopes, T cell epitopes, and B-cell epitopes.

By “immunogenicity” and grammatical equivalents herein is meant the degree of an immune response, including but not limited to production of neutralizing and non-neutralizing antibodies, formation of immune complexes, complement activation, mast cell activation, inflammation, and anaphylaxis. Immunogenicity is species-specific. In a preferred embodiment, immunogenicity refers to immunogenicity in humans. In an alternate embodiment, immunogenicity refers to immunogenicity in rodents, (rats, mice, hamster, guinea pigs, etc.), primates, farm animals (including sheep, goats, pigs, cows, horses, etc.), and domestic animals, (including cats, dogs, rabbits, etc).

By “enhanced immunogenicity” and grammatical equivalents herein is meant an increased ability to activate the immune system, when compared to a parent protein. For example, a variant protein can be said to have “enhanced immunogenicity” if it elicits neutralizing or non-neutralizing antibodies in higher titer or in more subjects than the parent protein. In a preferred embodiment, the probability of raising neutralizing antibodies is increased by at least 5%, with at least 2-fold or 5-fold increases being especially preferred. For example, if a parent protein produces an immune response in 10% of subjects, a variant with enhanced immunogenicity would produce an immune response in at least 10.5% of subjects, with more than 20% or more than 50% being especially preferred. A variant protein also can be said to have “increased immunogenicity” if it shows increased binding to one or more MHC alleles or if it induces T cell activation in a increased fraction of subjects relative to the parent protein. In a preferred embodiment, the probability of T cell activation is increased by at least 5%, with at least 2-fold or 5-fold increases being especially preferred.

By “reduced immunogenicity” and grammatical equivalents herein is meant a decreased ability to activate the immune system, when compared to a parent therapeutic agent. For example, if the therapeutic agent is protein, a variant protein can be said to have “reduced immunogenicity” if it elicits neutralizing or non-neutralizing antibodies in lower titer or in fewer subjects than the parent protein. In a preferred embodiment, the probability of raising neutralizing antibodies is decreased by, for example, at least 5%, with at least 50% or 90%. For example, if a parent protein produces an immune response in 10% of subjects, a variant with reduced immunogenicity would produce an immune response in not more than 9.5% of subjects, with less than 5% or less than 1% being especially preferred. A variant protein also can be said to have “reduced immunogenicity” if it shows decreased binding to one or more MHC alleles or if it induces T cell activation in a decreased fraction of subjects relative to the parent protein. In a preferred embodiment, the probability of T cell activation is decreased by at least 5%, with at least 50% or 90% decreases being especially preferred.

By “matrix method” and grammatical equivalents thereof herein is meant a method for calculating peptide—MHC affinity in which a matrix is used that contains a score for one or more possible residues at one or more positions in the peptide, interacting with a given MHC allele. The binding score for a given peptide—MHC interaction is obtained by summing the matrix values for the amino acids observed at each position in the peptide.

By “MHC-binding agretopes” and grammatical equivalents herein is meant peptides that are capable of binding to one or more class I or class II MHC alleles with appropriate affinity to enable the formation of MHC-peptide—T cell receptor complexes and subsequent T cell activation. Class II MHC-binding epitopes are linear peptide sequences that comprise at least approximately 9 residues.

By “parent protein” as used herein is meant a protein that is modified to generate a variant protein. Said parent protein may be a wild-type or naturally occurring protein, a variant or engineered version of a naturally occurring protein, or a de novo engineered protein. “Parent protein” may refer to the protein itself, compositions that comprise the parent protein, or any amino acid sequence that encodes it.

By “subject” herein is meant humans and other animals, particularly vertebrates, and more particularly mammals. Exemplary mammals include humans, rodents, (rats, mice, hamster, guinea pigs, etc.), primates, farm animals (including sheep, goats, pigs, cows, horses, etc.), and domestic animals, (including cats, dogs, rabbits, etc).

“Amino acid” also includes but natural and synthetic amino acids and both D- and L-amino acids may be utilized. For example, homo-phenylalanine, citrulline, and noreleucine are considered amino acids for the purposes of the invention.

By “predictive” herein is meant the ability of a system or assay to act as a surrogate for in vivo immunogenicity and recapitulate or mimic the immunogenic outcome or response of therapeutic administration in a vertebrate in the absence of actual administration. That is, a system or assay is predictive of vertebrate immunogenicity if the system can accurately demonstrate that the protein antigen would have or would not have elicited an immunogenic response had it been administered.

By “protein” herein is meant at least two covalently attached amino acids, which includes proteins, polypeptides, oligopeptides and peptides. The protein may be made up of naturally occurring amino acids and peptide bonds, or synthetic peptidomimetic structures, i.e., “analogs” such as peptoids (see Simon et al., Proc. Natl. Acad. Sci. U.S.A. 89(20:9367-71 (1992)), generally depending on the method of synthesis.

By “protein properties” herein is meant, biological, chemical, and physical properties including, but not limited to, enzymatic activity or specificity (including substrate specificity, kinetic association and dissociation rates, reaction mechanism, and pH profile), stability (including thermal stability, stability as a function of pH or solution conditions, resistance or susceptibility to ubiquitination or proteolytic degradation), solubility (including susceptibility to aggregation and crystallization), binding affinity or specificity (to one or more molecules including proteins, nucleic acids, polysaccharides, lipids, and small molecules), oligomerization state, dynamic properties (including conformational changes, allostery, correlated motions, flexibility, rigidity, folding rate), subcellular localization, ability to be secreted, ability to be displayed on the surface of a cell, susceptibility to co- or posttranslational modification (including N- or C-linked glycosylation, lipidation, and phosphorylation), ammenability to synthetic modification (including PEGylation, attachment to other molecules or surfaces), and ability to induce altered phenotype or changed physiology (including cytotoxic activity, immunogenicity, toxicity, ability to signal, ability to stimulate or inhibit cell proliferation, ability to induce apoptosis, and ability to treat disease).

By “T cell epitope” and grammatical equivalents herein is meant a residue or set of residues that are capable of being recognized by one or more T cell receptors. As is known in the art, T cells recognize linear peptides that are bound to MHC molecules.

By “treatment” herein is meant to include therapeutic treatment, as well as prophylactic, or suppressive measures for the disease or disorder. Thus, for example, successful administration of a therapeutic prior to onset of the disease may result in treatment of the disease. As another example, successful administration of a therapeutic after clinical manifestation of the disease to combat the symptoms of the disease comprises “treatment” of the disease. “Treatment” also encompasses administration of a therapeutic after the appearance of the disease in order to eradicate the disease. Successful administration of an agent after onset and after clinical symptoms have developed, with possible abatement of clinical symptoms and perhaps amelioration of the disease, further comprises “treatment” of the disease. Those “in need of treatment” include mammals already having the disease or disorder, as well as those prone to having the disease or disorder, including those in which the disease or disorder is to be prevented.

By “variant nucleic acids” and grammatical equivalents herein is meant nucleic acids that encode variant proteins of the invention. Due to the degeneracy of the genetic code, an extremely large number of nucleic acids may be made, all of which encode the variant proteins of the present invention, by simply modifying the sequence of one or more codons in a way that does not change the amino acid sequence of the variant protein.

By “variant proteins” and grammatical equivalents thereof herein is meant non-naturally occurring proteins which differ from a wild type or parent protein by at least 1 amino acid insertion, deletion, or substitution. Variant proteins are characterized by the predetermined nature of the variation, a feature that sets them apart from naturally occurring allelic or interspecies variation. Variant proteins typically either exhibit biological activity that is comparable to the parent protein or have been specifically engineered to have alternate biological properties. The variant proteins may contain insertions, deletions, and/or substitutions at the N-terminus, C-terminus, or internally. In a preferred embodiment, variant proteins have at least 1 residue that differs from the parent protein sequence, with at least 2, 3, 4, or 5 different residues being more preferred. Variant proteins may contain further modifications, for instance mutations that alter stability or solubility or which enable or prevent posttranslational modifications such as PEGylation or glycosylation. Variant proteins may be subjected to co- or post-translational modifications, including but not limited to synthetic derivatization of one or more side chains or termin, glycosylation, PEGylation, circular permutation, cyclization, fusion to proteins or protein domains, and addition of peptide tags or labels.

By “wild type or wt” and grammatical equivalents thereof herein is meant an amino acid sequence or a nucleotide sequence that is found in nature and includes allelic variations; that is, an amino acid sequence or a nucleotide sequence that has not been intentionally modified.

By “ex vivo” and grammatical equivalents is meant outside of an animal body. Ex vivo assays may use cells, organs, purified proteins, mixed proteins, etc.

Therapeutic Agents

As discussed above, a “therapeutic agent” is a compound capable of having a physiological effect on a subject in response to a disease state or disorder. Therapeutic agents are typically used to treat a subject in need of treatment.

Therapeutic agents can include a protein. Suitable proteins include, but are not limited to, industrial, pharmaceutical, and agricultural proteins, including ligands, cell surface receptors, antigens, antibodies, cytokines, hormones, transcription factors, signaling modules, cytoskeletal proteins and enzymes. Non-limiting examples of such therapeutic proteins include alpha-galactosidase, adenosine deamidase, arginase, asparaginase, bone morphogenic protein-7, ciliary neurotrophic factor, DNase, erythropoietin, factor IX, factor VIII, follicle stimulating hormone, glucocerebrocidase, gonadotrophin-releasing hormone, granulocyte-colony stimulating factor, granulocyte-macrophage-colony stimulating factor, growth hormone, growth hormone releasing hormone, human chorionic gonadotrophin, insulin, interferon alpha, interferon beta, interferon gamma, interleukin-2, interleukin-3, interleukin-1 1, salmon calcitonin, staphylokinase, streptokinase, tissue plasminogen activator, and thrombopoietin. The parent protein may also comprise an extracellular domain of a receptor, including but not limited to CD4, interleukin-1 receptor, tumor necrosis factor receptors, and antibodies (including a murine, chimeric, humanized, camelized, llamalized, single chain, or fully human antibodies). Proteinaceous therapeutic agents can be naturally occurring or synthetic.

In another embodiment, the therapeutic protein is a toxin that is used for therapeutic purposes. Exemplary therapeutic toxin parent proteins include but are not limited to botulinum toxin, ricin, and tetanus toxin.

In a further embodiment, the therapeutic protein is a designed or engineered protein that is being developed or used as a therapeutic. Such parent proteins include, but are not limited to, fusion proteins, proteins comprising one or more point mutations, chimeric proteins, truncated proteins, and the like. The therapeutic protein can also be an antibody.

In another embodiment, one class of therapeutic proteins is the interferons. Interferons (IFNs) are a well-known family of cytokines possessing a range of biological activities including antiviral, anti-proliferative, and immunomodulatory activities. Interferons have demonstrated utility in the treatment of a variety of diseases, and are in widespread use for the treatment of multiple sclerosis and viral hepatitis.

Interferons include a number of related proteins, such as interferon-alpha (IFN-α), interferon-beta (IFN-β), interferon-gamma (IFN-γ) interferon-kappa (IFN-κ, also known as interferon-epsilon or IFN-ε), interferon-tau (IFN-τ), and interferon-omega (IFN-ω). These interferon proteins are produced in a variety of cell types: IFN-α (leukocytes), IFN-β (fibroblasts), IFN-γ (lymphocytes), IFN-ε or κ (keratinocytes), IFN-ω (leukocytes) and IFN-τ (trophoblasts). IFN-α, IFN-β, IFN-ε or κ, IFN-ω, and IFN-τ are classified as type I interferons, while IFN-γ is classified as a type 11 interferon. Interferon alpha is encoded by a multi-gene family, while the other interferons appear to each be coded by a single gene in the human genome. Furthermore, there is some allelic variation in interferon sequences among different members of the human population.

Type-I interferons all appear to bind a common receptor, type I IFN-R, composed of IFNAR1 and IFNAR2 subunits. The exact binding mode and downstream signal transduction cascades differ somewhat among the type I interferons. However, in general, the JAK/STAT signal transduction pathway is activated following binding of interferon to the interferon receptor. STAT transcription factors then translocate to the nucleus, leading to the expression of a number of proteins with antiviral, antineoplastic, and immunomodulatory activities.

The properties of naturally occurring type I interferon proteins are not optimal for therapeutic use. Type I interferons induce injection site reactions and a number of other side effects. They are highly immunogenic, eliciting neutralizing and non-neutralizing antibodies in a significant fraction of subjects. Interferons are poorly absorbed from the subcutaneous injection site and have short serum half-lives. Finally, type I interferons do not express solubly in prokaryotic hosts, thus necessitating more costly and difficult refolding or mammalian expression protocols.

Correlating MHC Alleles with Immunogenicity of Therapeutic Agents

In one aspect, the present application is directed to methods of correlating at least one MHC allele with the immunogenicity of a therapeutic agent. At least some of the MHC alleles expressed by each of a plurality of subjects are determined. The therapeutic agent is then administered to the plurality of subjects, and the immunogenic response of each of the plurality of subjects to the therapeutic agent is measured. The MHC allele is compared with the immunogenic response of the plurality of subject expressing the allele to correlate at least one allele with the immunogenicity of a therapeutic agent.

Therapeutic agents may be administered to a subject by any methods known in the art. By way of example and not limitation, therapeutic agents can be administered by various in vivo methods including, but not limited to, orally, parenterally, subcutaneously, intravenously, and intraperitoneally. The therapeutic agent can be administered as part of a therapeutic or vaccination protocol. The method of administration depends on variables such as the therapeutic agent, the symptoms of the individual, and the disease or disorder being treated.

The immunogenic response is then measured. There are a wide variety of factors which contribute to altered immunogenicity, including the protein sequence and glycosylation state; the route and frequency of administration; the presence of adjuvants, particulates and/or aggregates; the presence or absence of non-human sequences; and the presence or absence of particular class I or class II MHC agretopes, T and B cell epitopes, etc.

The immunogenic response can be measured by any method known in the art. For example, a tissue or fluid of the subject (such as blood) can be obtained from the subject. Molecules implicated in upregulation of an immune response to the therapeutic agent can then be detected. For example, in certain embodiments, antibodies to the therapeutic agent can be detected. In other embodiments, other immune molecules that are up-regulated or down-regulated in response to an antigen can be detected.

Numerous methods of detecting molecules resulting from an immune response may be used. For example, in certain non-limiting examples antibodies to a therapeutic agent can be detected by a secondary molecule recognizing the antibody. In other non-limiting embodiments, secondary molecules to an immune response molecule can also be detected.

Measuring the immune response can make use of a labeled molecule. In general, labels may be either direct or indirect detection labels, sometimes referred to herein as “primary” and “secondary” labels. By “detection label” or “detectable label” herein is meant a moiety that allows detection. Accordingly, detection labels may be primary labels (i.e. directly detectable) or secondary labels (indirectly detectable; this is analogous to a “sandwich” type assay). In general, labels fall into four classes: a) isotopic labels, which may be radioactive or heavy isotopes; b) magnetic, electrical, thermal labels; c) colored or luminescent dyes or moieties; and d) binding partners. Labels can also include enzymes (horseradish peroxidase, etc.) and magnetic particles.

In one embodiment, the detection label is a primary label. A primary label is one that may be directly detected, such as a fluorophore. Preferred labels include chromophores or phosphors but are preferably fluorescent dyes or moieties. Fluorophores may be either “small molecule” fluores, or proteinaceous fluores. Suitable dyes for use in the invention include, but are not limited to, fluorescent lanthanide complexes, including those of Europium and Terbium, fluorescein, rhodamine, tetramethylrhodamine, eosin, erythrosin, coumarin, methyl-coumarins, quantum dots (also referred to as “nanocrystals”: see U.S. Ser. No. 09/315,584, hereby incorporated by reference), pyrene, Malacite green, stilbene, Lucifer Yellow, Cascade Blue®, Texas Red, Cy dyes (Cy3, Cy5, etc.), alexa dyes, phycoerythin, bodipy, and others described in the 6th Edition of the Molecular Probes Handbook by Richard P. Haugland, incorporated entirely by reference.

In other embodiments, a secondary label is used. A secondary label is one that is indirectly detected; for example, a secondary label can bind or react with a primary label for detection, can act on an additional product to generate a primary label (e.g. enzymes), or may allow the separation of the compound comprising the secondary label from unlabeled materials, etc. Secondary labels include, but are not limited to, one of a binding partner pair; chemically modifiable moieties; nuclease inhibitors, enzymes such as horseradish peroxidase, alkaline phosphatases, lucifierases, cell surface markers, etc.

In certain embodiments, the secondary label is a binding partner pair. For example, the label may be a hapten or antigen, which will bind its binding partner. For example, suitable binding partner pairs include, but are not limited to: antigens and antibodies (including fragments thereof (FAbs, etc.)); proteins and small molecules (including biotin/streptavidin); enzymes and substrates or inhibitors; other protein-protein interacting pairs; receptor-ligands; and carbohydrates and their binding partners. Exemplary binding partner pairs include, but are not limited to, biotin (or imino-biotin) and streptavidin, digeoxinin and Abs, and Prolinx reagents. In one embodiment, the binding partner may be attached to a solid support to allow separation of components containing the label and those that do not.

In a preferred embodiment, the binding partner pair includes a primary detection label and an antibody that will specifically bind to the primary detection label. By “specifically bind” herein is meant that the partners bind with specificity sufficient to differentiate between the pair and other components or contaminants of the system. The binding should be sufficient to remain bound under the conditions of the assay, including wash steps to remove non-specific binding. In some embodiments, the dissociation constants of the pair are less than about 10⁴-10⁶ M⁻¹, with less than about 10⁵-10⁹ M⁻¹, being preferred and less than about 10⁷-10⁹ M⁻¹ being particularly preferred.

In another embodiment, the secondary label is a chemically modifiable moiety. In this embodiment, labels comprising reactive functional groups are incorporated into the test molecule. The functional group can then be subsequently labeled (e.g. either before or after the assay) with a primary label. Suitable functional groups include, but are not limited to, amino groups, carboxy groups, maleimide groups, oxo groups and thiol groups, with amino groups and thiol groups being particularly preferred. For example, primary labels containing amino groups may be attached to secondary labels comprising amino groups, for example using linkers as are known in the art; for example, homo-or hetero-bifunctional linkers as are well known (see 1994 Pierce Chemical Company catalog, technical section on cross linkers, pages 155-200, incorporated by reference).

After the immunogenic response is detected, the immunogenic response is compared with the MHC alleles of the subjects to determine a correlation between the alleles and the subjects. In certain embodiments, for example, an increased immune response against a specific therapeutic agent corresponds to specific MHC alleles, or specific MHC subtypes of alleles. The correlation can be between a therapeutic agent and a single MHC allele, multiple MHC alleles at different MHC loci, an MHC subtype or allotype, or a combination thereof.

Determining the Immunological Compatibility of a Subject with a Therapeutic Agent

Immunological compatibility of a subject with a therapeutic agent can also be determined. Each subject has a defined group of MHC alleles. At least one MHC allele, subtype, or allotype is determined. One or more of the MHC alleles, subtypes, or allotypes expressed by the subject is compared to an immunological correlation between said MHC allele and the immunogenicity of the therapeutic agent to determine the immunological compatibility of the subject with the therapeutic agent.

The present invention can form part of a clinical trial. Clinical trial enrollees and subjects receiving certain therapeutics are constantly monitored for immunogenicity throughout all phases of a protein therapeutic clinical trial and post-regulatory approval administration of the therapeutic to the subject. Monitoring immunogenicity typically includes surveying for the presence of anti-therapeutic antibodies in subject sera using methods such as ELISA, Biacore® and radio-immunoprecipitation (RIP). While the modes of detection are different, all three methods capture antibodies by binding to the purified therapeutic protein.

In Vitro Vaccination

Methods of detecting immunogenicity of a therapeutic agent include in vitro vaccination (IVV). In a preferred embodiment, the method comprises measuring activation of reactive T cells using in vitro vaccination (IVV) with antigen presenting cells (APC) and CD4+ T cells from subject peripheral blood mononuclear cells (PBMCs). The PBMCs may be isolated prior to enrollment in a clinical trial, prior to said therapeutic exposure and/or after exposure to the therapeutic.

Typically, a significant lag time of about one to six months may exist between therapeutic administration and anti-therapeutic antibody detection. During this period, antigen-reactive CD4+ T cells are proliferating and accumulating in response to the therapeutic as a prelude to the onset of Cl.

The present invention circumvents the-above technical feasibility problems, and describes an “in vitro vaccination” technology (IVV) amenable to high throughput that may be used to test subjects pre-administration and during administration of a therapeutic that has potential immunogenic properties. Importantly, the invention can be performed long before detection of anti-therapeutic antibodies is possible, and in some embodiments can be performed prior to administration.

Previously described assays include those disclosed in Meidenbauer, N., Harris, D. T., Spitler, L. E., Whiteside, T. L., 2000, entirely incorporated by reference. Generation of PSA-reactive effector cells after vaccination with a PSA-based vaccine in subjects with prostate cancer. Prostate 43, 88-100 and Schultes, B. C and Whiteside, T. L., 2003. Monitoring of Immune Responses to CA125 with an IFN-γ ELISPOT Assay. J. Immunol. Methods 279, 1-15, entirely incorporated by reference.

There are different ways to prime the T cells in vitro. The antigen presenting cells (APCs) may be loaded with individual peptides, and selected T cells tested with the same peptides. In a preferred embodiment, the T cells can be primed with a combination of several peptides, and then tested with individual ones. In a preferred embodiment, the T cells can be selected with multiple rounds of stimulation with APCs loaded with proteins, and then tested with individual peptides from that protein to identify physiologically relevant epitopes.

Delineating potential immunogenic T cell epitopes within intact proteins is usually carried out by making overlapping synthetic peptides spanning the protein's sequence and using these peptides in T cell proliferation assays (see Stickler, M M, Estell, D A, Harding, F A “CD4+ T-Cell Epitope Determination Using Unexposed Human Donor Peripheral Blood Mononuclear Cells” J. Immunotherapy, 23, 654-660 (2000), entirely incorporated by reference). Uptake of peptides for MHC presentation by the APC may not be required since sufficient empty MHC class If molecules generally exist on the surface of most APC and bind sufficient quantity of peptide. While uptake and presentation of antigens derived from intact protein in these in vitro assays can be less efficient in the absence of receptor-mediated endocytosis, the use of intact protein is beneficial because the use of intact proteins will more closely mimic the physiological antigen processing pathway, thereby reducing the number of false immunogenic positives.

In a preferred embodiment of an IVV T cell assay, a DNA construct is constructed that includes attaching a tag (e.g., Myc, His, S-tag, Flag) to the protein. A preferred tag should itself be non-immunogenic and will have commercially available mouse monoclonal antibodies. In addition, a humanized anti-tag antibody is used. The humanized anti-tag antibody is generated preferably by grafting the mouse variable regions onto a human IgG scaffold or by removing T-helper cell epitopes. The protein-tag-antibody complex is introduced into a CD4(+) T cell assay in which the complex will target an antigen presenting cell (APC: e.g., dendritic cell or macrophage) via cell surface Fcγ receptors.

In another preferred embodiment, reactive polyclonal T cell populations expanded after multiple rounds of re-stimulation in the presence of MHC-restricted antigen are used to map the immunodominant epitopes present within the protein of interest.

The IVV assay may be performed using the following steps: (1) therapeutic agent is introduced to the antigen presenting cell (APC) and appropriate conditions found to stimulate efficient uptake and processing, (2) the APC with multiple MHC-restricted epitopes will stimulate initially naive T cells, (3) multiple rounds of T cell re-stimulation will take place to ensure a large population of reactive polyclonal T cells, (4) this pool of reactive T cells is divided into smaller amounts, 5) potential peptide epitopes from the full length protein are synthesized based on either prediction or from an overlapping peptide library, 6) each peptide is tested for T cell reactivity for the samples from step (4) above. The testing may use, for example, the EliSPOT method. The IVV method is sensitive enough to detect the presence of antigen-reactive CD4+ T cells in subject sera prior to a build up of clinically relevant antibodies.

Any peptide causing further T cell activation is most likely part of an epitope originally MHC-restricted after antigen processing in step (1) above.

The present invention provides in vitro testing of T cell activation by endogenous or foreign proteins or peptides. CD4+ T cells are activated in vitro by repeated cycles of exposure to the antigen presenting cells loaded with whole proteins or peptides. T cells undergo negative selection during their development to minimize the number that are reactive to self-antigens. Hence, the vast majority of naive T cells may not be reactive to many therapeutic proteins of human origin, and in vitro immunogenicity testing in that capacity with naive T cells may hinder the discovery of potential MHC-binding epitopes. Conditions for in vitro activation of T cells that mimic in vivo activation are a preferred embodiment as it allows for further optimization. Antigen presenting cells loaded with the test antigen may be preserved frozen or freshly prepared prior to each induction, and aliquots thawed prior to each T cell activation. This method of the present invention allows consistency regarding the APCs used for the various cycles of T cell activation. In a preferred embodiment, either peptides or whole proteins may be tested.

In a preferred embodiment, in vitro assessment of immunogenicity of endogenous or foreign proteins in humans may be done. In many instances, a lack of measurable T cell response could be the result of too few T cells present with the appropriate specificity for the given MHC/peptide complex. Moreover, because T cells undergo negative selection during their development to reduce the number of T cells reactive to self-antigens, human proteins, such thrombopoietin, may not stimulate sufficient numbers of naive T cells to be statistically significant.

In a preferred embodiment, it is desirable to increase the population of reactive CD4+ T cells prior to the activation assay. As is known in the art, dendritic cells may be produced from proliferating dendritic cell precursors. (See, e.g., USSN 2002/0085993, U.S. Pat. Nos. 5,994,126; 6,274,378; 5,851,756; and WO93/20185, all entirely incorporated by reference.) Dendritic cells pulsed with proteins or peptides are co-cultured with CD4+ T cells. Rounds of T cell proliferation in the presence of antigen presenting dendritic cells simulate in vivo clonal expansion (“in vitro vaccination”). See for example, WO9833888, entirely incorporated by reference. The number of rounds required is empirically determined based on signaling.

In a preferred embodiment, full length and truncated (receptor-binding domain) proteins may be tested with the IVV technology of the present invention. Peptides derived from the protein sequence will also be evaluated, and the necessary number of exposures (dendritic cells vs. T cells) to obtain sufficient and measurable T cell activation determined. The proteins/peptides is tested with cells from several different donors (different alleles). Preferably, APCs are be dendritic cells isolated either directly from subject PBMC or differentiated from subject monocytes. Antigen-dependent activation of CD4+ T-helper cells is required prior to the sustained production of the antibody isotype most relevant to Cl.

Enzymatic processing of exogenous antigens by professional antigen presenting cells (APC) provides a pool of potentially antigenic peptides from which proteins encoded in the Major Histocompatibility Complex (MHC class II molecules) are drawn from for loading and presentation to CD4+ T cells. T cells expressing the appropriate T cell receptor with basal affinity for the MHC/peptide complex on the APC surface activate and proliferate in response to the interaction. T cells isolated from “unprimed” individuals that have had little or no prior exposure to a particular antigen are said to be “naive”. During the development of T cells, positive and negative selection may take place. Positive selection ensures that the individual's T cell population expresses viable T cell receptors while negative selection minimizes the number of high affinity self-reactive T cells.

For the purposes of measuring ex vivo T cell activation in response to self antigen, in vivo negative selection may hinder the measurement due to low numbers of T cells available to react and thereby lowering the confidence that any lack of T cell activation really signifies the absence of MHC binding epitopes. Multiple rounds of T cell re-stimulation and proliferation in the presence of antigen-loaded professional antigen presenting cells (e.g., dendritic cells) may produce an expanded polyclonal population of T cells reactive to MHC epitope(s) created by the antigen.

In addition, antigen-specific IVV with PBMCs from subject sera may be applied prior to enrollment to predict immunogenicity. Screening out individuals that could react adversely to the therapeutic during the trial may consequently improve the trial's overall likelihood of clinical success. Clinical trial enrollees are constantly monitored for immunogenicity throughout all phases of a protein therapeutic clinical trial and the method of the invention may be used after administration of a therapeutic either once or periodically throughout the administration of the therapeutic. Post administration testing may identify immunogenic responses prior to detecting clinical sequelae in the subject, and the therapeutic administration can be stopped to prevent further immunogenic responses.

In a further aspect, the invention is directed to a method of selecting a therapeutic agent for a subject in need of treatment. The immunological compatibility of the subject with the therapeutic agent is determined. The subject is then treated with the therapeutic agent if the subject is immunologically compatible with the therapeutic agent. Alternatively, the subject can be treated with another therapy, such as an immunosuppressive therapy, if the subject is has immunological compatibility with the therapeutic agent.

Designing a Therapeutic Agent with Reduced Immunogenicity

In a further aspect, the present invention is directed to a method of designing a therapeutic agent with reduced immunogenicity for a subject by determining the immunological compatibility of a subject and a therapeutic agent and designing a derivative having reduced immunogenicity to the therapeutic agent.

Proteins with desired immunological and functional properties can serve as valuable therapeutics or vaccines. However, efforts to modulate immunogenicity while conserving function have met with only limited success. Mutations that confer desired immunological properties and mutations that confer desired functional properties are both typically rare, and so mutations that confer both sets of properties are even less frequent. As a result, proteins that are engineered for reduced or increased immunogenicity often lack desired functional properties, and proteins that are designed for improved function may possess unwanted immunogenicity. It is possible to screen variants with altered immunogenicity for function, or to screen functional variants for desired immunological properties. The present invention is also related to computational methods, comprising computational protein design algorithms and computational immunogenicity filters, which can analyze protein sequences to select smaller libraries of protein sequences. For example, if a protein with reduced immunogenicity is desired, computational methods may be used to identify and replace residues that promote immunogenicity with alternate residues that maintain the native structure and function of the protein; thereby generating a functional, less immunogenic variant. If a protein with increased immunogenicity is desired, computational methods may be used to introduce one or more epitopes or agretopes while maintaining desired functional properties. The resulting protein libraries are greatly enriched for variants that possess desired functional and immunological properties.

The present invention also comprises three basic approaches to generate proteins with desired functional and immunological properties: (1) use a computational protein design algorithm to identify a set of proteins that are predicted to possess desired functional properties, and then use a computational immunogenicity filter to identify the subset of proteins that also possess desired immunological properties; (2) use a computational protein design algorithm to identify a set of proteins that are predicted to possess desired immunological properties, and then use a computational immunogenicity filter to identify the subset of proteins that also possess desired functional properties; or (3) use a computational algorithm comprising both protein design and immunogenicity filter algorithms that generates proteins with desired functional and immunological properties.

In a preferred embodiment, the computational method used to identify protein sequences with desired functional properties is Protein Design Automation® (PDA®) technology, as is described in U.S. Pat. Nos. 6,188,965; 6,269,312; 6,403,312; WO98/47089 and U.S. Ser. Nos. 09/058,459, 09/714,357, 09/812,034, 09/827,960, 09/837,886, 09/877,695,10/071,85909/419,351, 09/782,004 and 09/927,790, 60/347,772,10/101,499, and 10/218,102; and PCT/US01/218,102 and U.S. Ser. No. 10/218,102, U.S. Ser. No. 60/345,805; U.S. Ser. No. 60/373,453 and U.S. Ser. No. 60/374,035, all entirely incorporated by reference. Briefly, PDA® technology may be described as follows. A protein structure (which may be determined experimentally, generated by homology modeling or produced de novo) is used as the starting point. The positions that are allowed to vary are then identified, which may be the entire sequence or subset(s) thereof. The amino acids that is considered at each variable position are selected. Optionally, each amino acid residue may be represented by a discrete set of allowed conformations, called rotamers. Interaction energies are calculated using a scoring function between (1) each allowed residue or rotamer at each variable position and the backbone, (2) each allowed residue or rotamer at each variable position and each non-variable residue (if any), and (3) each allowed residue or rotamer at each variable position and each allowed residue or rotamer at each other variable position. Combinatorial search algorithms, typically DEE and Monte Carlo, are used to identify the optimum amino acid sequence and additional low energy sequences. The resulting sequences may be generated experimentally or subjected to further computational analysis.

In a preferred embodiment peptides responsible for protein immunogenicity can be detected using in vitro T cell activation assays. Clinical validation for the association of MHC-binding epitopes and immunogenicity of therapeutic proteins can then be modified to alter the MHC-binding epitopes to obtain the desired results. Peptides selected in the assay can be engineering to modulate the desired immunogenicity.

In another preferred embodiment an ex vivo T cell activation assay is developed that mimics immunogenicity in vivo (ex vivo vaccination, or EVV). The assay to identify immunogenic regions in a protein can use cells that have been primed in vivo (derived from blood from subjects that have developed neutralizing antibodies to a given protein) or cells primed ex vivo to mimic the in vivo situation.

The methods described herein may be applied to any protein. In a preferred embodiment, the three-dimensional structure of the parent protein is known or may be generated using experimental methods, homology modeling, or de novo fold prediction methods. However, in some embodiments, it is possible to generate variants without a three-dimensional structure of the parent protein.

Parent Proteins and Peptides

The parent protein that is derived to form a variant protein can be a therapeutic protein, as discussed herein.

In an additional embodiment, the parent protein is a protein associated with an allergen, viral pathogen, bacterial pathogen, other infectious agent, or cancer. Variants of such parent proteins may serve as vaccines that are effective against allergens, bacterial pathogens, viral pathogens and tumors (see for example, U.S. Pat. Nos. 6,322,789; 6,329,505; and WO 01/41788; WO 01/41799; WO 01/42267; WO 01/42270; and WO 01/45728, all entirely incorporated by reference).

Preferred allergen-derived parent proteins include but are not limited to proteins in chemical allergens, food allergens, pollen allergens, fungal allergens, pet dander, mites, etc (see Huby, R. D. et al., Toxicological Science, 55:235-246 (2000), entirely incorporated by reference).

Preferred viral pathogen-derived parent proteins include but are not limited to proteins expressed by Hepatitis A, Hepatitis B, Hepatitis C, poliovirus, HIV, herpes simplex I and II, small pox, human papillomavirus, cytomegalovirus, hantavirus, rabies, Ebola virus, yellow fever virus, rotavirus, rubella, measles virus, mumps virus, Varicella (i.e., chicken pox or shingles), influenza, encephalitis, Lassa Fever virus, etc.

Preferred bacterial pathogen-derived parent proteins include but are not limited to proteins expressed by the causative agent of Lyme disease, diphtheria, anthrax, botulism, pertussis, whooping cough, tetanus, cholera, typhoid, typhus, plague, Hansen's disease, tuberculosis (including multidrug resistant forms), staphylococcal infections, streptococcal infections, Listeria, meningococcal meningitis, pneumococcal infections, legionnaires' disease, ulcers, conjunctivitis, etc.

Additional parent proteins derived from infectious agents include but are not limited to proteins expressed by the causative agent of dengue fever, malaria, African Sleeping Sickness, dysentery, Rocky Mountain Spotted Fever, Schistosomiasis, Diarrhea, West Nile Fever, Leishmaniasis, Giardiasis, etc.

Preferred cancer-derived parent proteins include but are not limited to proteins expressed by solid tumors such as skin, breast, brain, cervical carcinomas, testicular carcinomas, etc., such as melanoma antigen genes (MAGE; see WO 01/42267); carcinoembryonic antigen (CEA; see WO 01/42270), prostate cancer antigens (see WO 01/45728 and U.S. Pat. No. 6,329,505), such as prostate specific antigen (PSA), prostate specific membrane antigen (PSM), prostatic acid phosphatase (PAP), and human kallikrein2 (hK2 or HuK2), and breast cancer antigens( (i.e., her2/neu; see AU 2087401), all entirely incorporated by reference. Additional cancer-derived proteins include proteins that are expressed in one or more of the following types of cancer: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastom, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma.

Identification of Immunogenic Sequences in the Parent Protein

In a preferred embodiment, after selection of a parent protein, the parent protein is analyzed to identify one or more immunogenic sequences. These sequences may be targeted for modification in order to confer reduced immunogenicity. Similarly, if enhancing immunogenicity is the goal, analysis of the immunogenic sequences in the parent protein may be used to suggest which classes of immunogenic sequences should be incorporated to increase immunogenicity. Finally, novel sequences including but not limited to those discovered using computational protein design methods may be analyzed for their potential to elicit an immune response using the methods described below.

Identification of Binding Sites for APC Receptors

Receptor mediated endocytosis delivers protein antigens to APCs far more effectively than pinocytosis does, thereby promoting immunogenicity. APCs express a wide variety of receptors, including receptors that bind antibodies, many cytokines and chemokines, and specific glycoforms. Protein antigen interaction with APC cell surface receptors, such as the mannose receptor (Tan M C et al. Adv Exp Med Biol, 417: 171-174 (1997), entirely incorporated by reference), increases the efficiency of protein antigen uptake.

In a preferred embodiment, the parent protein is analyzed to determine whether it could act as a ligand for any of the receptors that are present on the surface of APCs. For example, binding assays may be conducted using the parent protein and one or more types of APCs. Furthermore, a number of proteins are already known to bind to one or more receptors on the surface of one or more types of APCs. Receptors that are present on APCs include, but are not limited to, Toll-like receptors (for example receptors for lipopolysaccharide, bacterial proteoglycans, unmethylated CpG motifs, and double stranded RNA), cytokine receptors (for example CD40, Fas, OX40L, gp130, LIFR, and receptors for interferon alpha, interferon-beta, interleukin-1, interleukin-3 interleukin-4, interleukin-10, interleukin-12, tumor necrosis factor alpha), and Fc receptors (for example Fc gamma RI, Fc gamma RIII).

Identification of Residues that Promote Aggregation

Protein aggregation is often driven by the formation of intermolecular disulfide bonds or intermolecular hydrophobic interactions. Accordingly, free cysteines (that is, cysteines that are not participating in disulfide bonds) and solvent exposed hydrophobic residues often mediate aggregation.

In a preferred embodiment, biophysical characterization is performed to determine whether the parent protein is susceptible to aggregation. Methods for assaying for aggregation include, but are not limited to, size exclusion chromatography, dynamic light scattering, analytical ultracentrifugation, UV scattering, and decrease of protein amount or activity over time.

In an alternate preferred embodiment, the parent protein is analyzed to identify any free cysteine residues. This may be done, for example, by inspecting the three-dimensional structure or by performing a sequence alignment and analyzing conservation patterns.

In another preferred embodiment, the parent protein is analyzed to identify any exposed hydrophobic residues. Hydrophobic residues include valine, leucine, isoleucine, methionine, phenylalanine, tyrosine, and tryptophan, and exposed hydrophobic residues are those hydrophobic residues whose side chains are significantly exposed to solvent. In a preferred embodiment, at least 30 Å² of solvent exposed area is present, with greater than 50 Å² or 75 Å² being especially preferred. In an alternate embodiment, at least 50% of the surface area of the side chain is exposed to solvent, with greater than 75% or 90% being preferred.

The isoelectric point or pi (that is, the pH at which the protein has a net charge of zero) of the protein may also affect solubility. As is known in the art, protein solubility is typically lowest when the pH is equal to the pi. Furthermore, proteins with net positive charge may interact with proteoglycans present at the injection site, which may potentially promote aggregation. Accordingly, in a preferred embodiment, the net charge of the parent protein is calculated at physiological pH.

Identification of Class I Antigen Processing Sites

Prior to binding class I MHC molecules, a protein antigen is “processed”, meaning that it is subjected to limited proteolytic cleavage in order to produce peptide fragments. The proteosome performs antigen processing for the class I pathway. Potential proteosomal cleavage sites may be identified by using any of a number of prediction algorithms (see for example Kutter, C., et al., J. Mol. Biol., 298:417-429 (2000) and Nussbaum, A. K., et al., Immunogenetics, 53:87-94 (2001), both entirely incorporated by reference).

Identification of Class II Antigen Processing Sites

Antigen processing also takes place prior to binding class II MHC molecules. A number of proteolytic enzymes participate in antigen processing for the class II pathway, including but not limited to cathepsins B, D, E, L and asparaginyl endopeptidase. Potential proteolytic cleavage sites may be identified, for example, as described by Schneider, S. C., et al., J. Immunol., 165:20-23 (2000); and by Medd and Chain, Cell Dev. Biol., 11:203-210 (2000), all entirely incorporated by reference.

Identification of Class I MHC-Binding Agretopes

Class I MHC molecules primarily bind fragments of intracellular proteins that are derived from infecting viruses, intracellular parasites, or internal proteins of the cell; proteins that are overexpressed in cancer cells are of special interest. The resulting peptide-MHC complexes are transported to the surface of the APC, where they may interact with T cells via TCRs. This is the first step in the activation of a cellular program that may lead to cytolysis of the APC, secretion of lymphokines by the T cell, or signaling to natural killer cells. The interaction with the TCR is dependent on both the peptide and the MHC molecule. MHC class I molecules show preferential restriction to CD8+ cells. (Fundamental Immunology, 4th edition, W. E. Paul, ed., Lippincott-Raven Publishers, 1999, Chapter 8, pp 263-285), entirely incorporated by reference.

The factors that determine the affinity of peptide-class I MHC interactions have been characterized using biochemical and structural methods, including sequencing of peptides and natural peptide libraries extracted from MHC proteins. Class I MHC ligands are mostly octa-or nonapeptides; they bind a groove in the class I MHC structure framed by two alpha helices and a beta pleated sheet. Specific pockets in the binding groove recognize a subset of residues in the peptide, called anchor residues, and these interactions confer some sequence selectivity.

Class I MHC molecules also interact with atoms in the peptide backbone. The orientation of the peptides is determined by conserved side chains of the MHC I protein that interact with the N- and C-terminal residues in the peptide.

Any of a number of methods may be used to identify potential class I MHC agretopes, including but not limited to the computational and experimental methods described below.

Rules for identifying MHC I binding sites have been described in Altuvia, Y., et al (1997) Human Immunology, 58:1-11; Meister, G E., et al (1995) Vaccine: 6:581-591; Parker, K. C., et al., (1994) J. Immunology, 152:163; Gulukota, K., et al., (1997) J. Mol. Biol., 267:1258-1267; Buus, S., (1999) Current Opinion Immunology, 11:209-213; all entirely incorporated by reference). Databases of MCH binding peptide, such as SYPEITHI and MHCPEP may also be used to identify potential MHC I binding sites (Rammensee, H-G., et al., (1999) Immunogenetics, 50:213-219; Brusic, V., et al., (1998) Nucleic Acids Research, 26:368-371), both entirely incorporated by reference. Other methods for identifying MHC binding motifs include allele-specific polynomial algorithms described by Fikes, J., et al., WO 01/41788, entirely incorporated by reference, neural net (Gulukota, K, supra), polynomial (Gulukota, K., supra) and rank ordering algorithms (Parker, K. C., supra).

Identification of Class II MHC-Binding Agretopes

Class II MHC molecules, which are related to class I MHC molecules, primarily present extracellular antigens. Relatively stable peptide-MHC complexes may be recognized by TCRs; this recognition event is required for the initiation of most antibody-based (humoral) immune responses. MHC class II molecules show preferential restriction to CD4+ cells (Fundamental Immunology, 4th edition, W. E. Paul, ed., Lippincott-Raven Publishers, 1999, Chapter 8, pp 263-285), entirely incorporated by reference.

The factors that determine the affinity of peptide-class II MHC interactions have been characterized using biochemical and structural methods. Peptides bind in an extended conformation bind along a groove in the class II MHC molecule. While peptides that bind class II MHC molecules are typically approximately 12-25 residues long, a nine-residue region is responsible for most of the binding affinity and specificity. The peptide binding groove can be subdivided into “pockets”, commonly named P1 through P9, where each pocket is comprises the set of MHC residues that interacts with a specific residue in the peptide. Between two and four of these positions typically act as anchor residues. As in the class I ligands, the non-anchoring amino acids play a secondary, but still significant role (Rammensee, H., et al., (1999) Immunogenetics, 50:213-219, entirely incorporated by reference). A number of polymorphic residues face into the peptide-binding groove of the MHC molecule. The identity of the residues lining each of the peptide-binding pockets of each MHC molecule determines its peptide binding specificity. Conversely, the sequence of a peptide determines its affinity for each MHC allele.

Several methods of identifying MHC-binding agretopes in protein sequences are known in the art and may be used, including but not limited to, those described in a recent review (Schirle et al. J. Immunol. Meth. 257: 1-16 (2001), entirely incorporated by reference) and those described below.

In one embodiment, structure-based methods are used. For example, methods may be used in which a given peptide is computationally placed in the peptide-binding groove of a given MHC molecule and the interaction energy is determined (for example, see WO 98/59244 and WO 02/069232, both entirely incorporated by reference). Such methods may be referred to as “threading” methods.

Alternatively, purely experimental methods may be used. Examples of physical methods include high affinity binding assays (Hammer, J., et al. (1993) Proc. Natl. Acad. Sci. USA, 91:4456-4460; Sarobe, P. et al. (1998) J. Clin. Invest., 102:1239-1248), T cell proliferation and CTL assays (WO 02/77187, Hemmer, B., et al., (1998) J. Immunol., 160:3631-3636); stabilization assays, competitive inhibition assays to purified MHC molecules or cells bearing MHC, or elution followed by sequencing (Brusic, V., et al., (1998) Nucleic Acids Res., 26:368-371), all entirely incorporated by reference.

In a preferred embodiment, potential MHC II binding sites are identified by matching a database of published motifs, such as SYFPEITHI (Rammensee, H., et al., (1999) Immunogenetics, 50:213-219, entirely incorporated by reference, or MHCPEP (Brusic, B., et al., supra).

Sequence-based rules for identifying MHC II binding sites, including but not limited to matrix method calculations, have been described in Sturniolo, T, et al. Nat. Biotechnol., 17:555-561 (1999); Hammer, J. et al., Behring. Inst. Mitt., 94:124-132 (1994); Hammer, J. et al., J. Exp. Med., 180:2353-2358 (1994); Mallios, R. R J. Com. Biol., 5:703-711. (1998); Brusic, V., et al., Bioinformatics, 14:121-130 (1998); Mallios, R. R. Bioinformatics, 15:432-439 (1999); Marshall, K.W., et al., J. Immunology, 154:5927-5933 (1995); Novak, E. J., et al., J. Immunology, 166:6665-6670 (2001); Cochlovius, B., et al., J. Immunology, 165:4731-4741 (2000); and by Fikes, J., et al., WO 01/41788), all entirely incorporated by reference.

In an especially preferred embodiment, the matrix method is used to calculate MHC-binding propensity scores for each peptide of interest binding to each allele of interest. The matrix comprises binding scores for specific amino acids interacting with the peptide binding pockets in different human class II MHC molecule. It is possible to consider all of the residues in each 9-mer window; it is also possible to consider scores for only a subset of these residues, or to consider also the identities of the peptide residues before and after the 9-residue frame of interest. The scores in the matrix may be obtained from experimental peptide binding studies, and, optionally, matrix scores may be extrapolated from experimentally characterized alleles to additional alleles with identical or similar residues lining that pocket. Matrices that are produced by extrapolation are referred to as “virtual matrices”. (See Sturniolo, T., Bono, E., Ding, J., Raddrizzani, L., Tuereci, O., Sahin, U., Braxenthaler, M., Gallazzi, F., Protti, M. P., Sinigaglia, F., and Hammer, J. (1999) “Generation of tissue-specific and promiscuous HLA ligand databases using DNA micro arrays and virtual HLA class II matrices” Nat. Biotech., 17, 555-61 (1999) entirely incorporated by reference). While the fundamental binding units of class I and class II MHC proteins are both 9 amino acids, it is also known that additional peptide flanking residues (PFRs) can influence T-cell recognition (via the TCR) of class II MHC-peptide complexes. (See, e.g., Arnold et al., 2002, J Immunology 169(2):739-49, entirely incorporated by reference.) Residues at positions P-1 (one position before the first MHC binding position) and P11 appear to be the most influential. In an alternative embodiment, residues at these positions can be engineered to reduce immunogenicity of a therapeutic protein.

Several methods may then be used to determine whether a given peptide will bind with significant affinity to a given MHC allele. In one embodiment, the binding score for the peptide of interest is compared with the binding propensity scores of a large set of reference peptides. Peptides whose binding propensity scores are large compared to the reference peptides are likely to bind MHC and may be classified as “hits.” For example, if the binding propensity score is among the highest 1% of possible binding scores for that allele, it may be scored as a “hit” at the 1% threshold. The total number of hits at one or more threshold values is calculated for each peptide. In some cases, the binding score may directly correspond with a predicted binding affinity. Then, a hit may be defined as a peptide predicted to bind with at least 100 μM or 1 μM or 100 nM affinity.

In a preferred embodiment, the number of hits for each 9-mer frame in the protein is calculated using one or more threshold values ranging from 0.5% to 10%. In an especially preferred embodiment, the number of hits is calculated using 1%, 3%, and 5% thresholds.

In a preferred embodiment, MHC-binding epitopes are identified as the 9-mer frames that bind to several class II MHC alleles. In an especially preferred embodiment, MHC-binding epitopes are predicted to bind at least 10 alleles at 5% threshold and/or at least 5 alleles at 1% threshold. Such 9-mer frames may be especially likely to elicit an immune response in many members of the human population.

In a preferred embodiment, MHC-binding epitopes are predicted to bind MHC alleles that are present in at least 0.01-10% of the human population. Alternatively, to treat conditions that are linked to specific class II MHC alleles, MHC-binding epitopes are predicted to bind MHC alleles that are present in at least 0.01-10% of the relevant subject population.

Data about the prevalence of different MHC alleles in different ethnic and racial groups has been acquired by groups such as the National Marrow Donor Program (NMDP); for example see Mignot et al. Am. J. Hum. Genet. 68: 686-699 (2001), Southwood et al. J. Immunol. 160: 3363-3373 (1998), Hurley et al. Bone Marrow Transplantation 25: 136-137 (2000), Sintasath Hum. Immunol. 60: 1001 (1999), Collins et al. Tissue Antigens 55: 48 (2000), Tang et al. Hum. Immunol. 63: 221 (2002), Chen et al. Hum. Immunol. 63: 665 (2002), Tang et al. Hum. Immunol. 61: 820 (2000), Gans et al. Tissue Antigens 59: 364-369, and Baldassarre et al. Tissue Antigens 61: 249-252 (2003), all entirely incorporated by reference.

In a preferred embodiment, MHC binding epitopes are predicted for MHC heterodimers comprising highly prevalent MHC alleles. Class II MHC alleles that are present in at least 10% of the US population include but are not limited to: DPA1*0103, DPA1*0201, DPB1*0201, DPB1*0401, DPB1*0402, DQA1*0101, DQA1*0102, DQA1*0201, DQA1*0501, DQB1*0201, DQB1*0202, DQB1*0301, DQB1*0302, DQB1*0501, DQB1*0602, DRA*0101, DRB1*0701, DRB1*1501, DRB1*0301, DRB1*0101, DRB1*1101, DRB1*1301, DRB3*0101, DRB3*0202, DRB4*0101, DRB4*0103, and DRB5*0101.

In a preferred embodiment, MHC binding epitopes are also predicted for MHC heterodimers comprising moderately prevalent MHC alleles. Class II MHC alleles that are present in 1% to 10% of the US population include but are not limited to: DPA1*0104, DPA1*0302, DPA1*0301, DPB1*0101, DPB1*0202, DPB1*0301, DPB1*0501, DPB1*0601, DPB1*0901, DPB1*1001, DPB1*1101, DPB1*1301, DPB1*1401, DPB1*1501, DPB1*1701, DPB1*1901, DPB1*2001, DQA1*0103, DQA1*0104, DQA1*0301, DQA1*0302, DQA1*0401, DQB1*0303, DQB1*0402, DQB1*0502, DQB1*0503, DQB1*0601, DQB1*0603, DRB1*1302, DRB1*0404, DRB1*0801, DRB1*0102, DRB1*1401, DRB1*1104, DRB1*1201, DRB1*1503, DRB1*0901, DRB1*1601, DRB1*0407, DRB1*1001, DRB1*1303, DRB1*0103, DRB1*1502, DRB1*0302, DRB1*0405, DRB1*0402, DRB1*1102, DRB1*0803, DRB1*0408, DRB1*1602, DRB1*0403, DRB3*0301, DRB5*0102, and DRB5*0202.

MHC binding epitopes may also be predicted for MHC heterodimers comprising less prevalent alleles. Information about MHC alleles in humans and other species can be obtained, for example, from the IMGT/HLA sequence database.

In an additional preferred embodiment, MHC-binding epitopes are identified as the 9-mer frames that are located among “nested” epitopes, or overlapping 9-residue frames that are each predicted to bind a significant number of alleles. Such sequences may be especially likely to elicit an immune response.

Identification of T Cell Epitopes

T cell epitopes overlap with MHC agretopes, as TCRs recognize peptides that are bound to MHC molecules. Accordingly, methods for the identification of MHC agretopes may also be used to identify T cell epitopes, and similarly the methods described below for the identification of T cell epitopes may also be used to identify MHC agretopes.

TCRs occur as either of two distinct heterodimers, alpha:beta or gamma:delta both of which are expressed with the non-polymorphic CD3 polypeptides gamma, delta, epsilon, zeta. The CD3 polypeptides, especially zeta and its variants, are critical for intracellular signaling. The alpha:beta TCR heterodimer expressing cells predominate in most lymphoid compartments and are responsible for the classical helper or cytotoxic T cell responses. In most cases, the alpha:beta TCR ligand is a peptide antigen bound to a class I or a class II MHC molecule (Fundamental Immunology, 4th edition, W. E. Paul, ed., Lippincott-Raven Publishers, 1999, Chapter 10, pp 341-367, entirely incorporated by reference).

Preferably, potential T cell epitopes are identified by matching a database of published motifs (Walden, P., (1996) Curr. Op. Immunol., 8:68-74, entirely incorporated by reference). Other methods of identifying T cell epitopes which are useful in the present invention include those described by Hemmer, B., et al. (1998) J. Immunol., 160:3631-3636; Walden, P., et al. (1995) Biochemical Society Transactions, 23; Anderton, S. M., et al., (1999) Eur. J. Immunol., 29:1850-1857; Correia-Neves, M., et al., (1999) J. Immunol., 163:5471-5477; Shastri, N., (1995) Curr. Op. Immunol., 7:258-262; Hiemstra, H. S., (2000) Curr. Op. Immunol., 12:80-84; and Meister, G. E., et al., (1995) Vaccine, 13:581-591, entirely incorporated by reference).

Identification of Antibody Epitopes

Antibody epitopes may be identified using any of a number of computational or experimental approaches. As is known in the art, antibody epitopes typically possess certain structural features, such as solvent accessibility, flexibility, and the presence of large hydrophobic or charged residues. Computational methods have been developed to predict the location of antibody epitopes based on sequence and structure (Parker et. al. Biochem. 25: 5425-5432 (1986) and Kemp et. al. Clin. Exp. Immunol. 124: 377-385 (2001), both entirely incorporated by reference). Experimental methods such as NMR and crystallography may be used to map antigen-antibody contacts. Also, mass spectrometry approaches have been developed (Spencer et. al. Proteomics 2: 271-279 (2002), entirely incorporated by reference). It is also possible to use mutagenesis-based approaches, in which changes in the antibody binding affinity of one or more mutant proteins is used to identify residues that confer antibody binding affinity.

Confirmation of Immunogenic Sequences

In a preferred embodiment, if computational methods were used to identify one or more immunogenic sequences, experimental methods are used to confirm the immunogenicity of the identified sequences prior to proceeding with the identification of variant proteins with modified immunogenicity. A number of methods, including but not limited to those described in Stickler et al. J. Immunol. 23: 654-660 (2000, entirely incorporated by reference) and below in the section “Assaying the immunogenicity of the variants” may be used.

Identifying Variants with Desired Immunological Properties

Variant proteins with reduced or enhanced immunogenicity, relative to the parent protein, may be generated by introducing modifications including but not limited to those described below. In general, methods for reducing immunogenicity will find use in the development of safer and more effective protein therapeutics, while methods for increasing immunogenicity will find use in the development of more effective protein vaccines.

Enhancing APC Uptake

In a preferred embodiment, the parent protein is modified to enhance uptake by APCs. This may be accomplished by increasing the oligomerization state or effective size of the protein. For example, covalent linkage to synthetic microspheres or other particulate matter may be used to enhance APC uptake (Gengoux and Leclerc, Int. Immunol. 7: 45-53 (1995), entirely incorporated by reference). Alternatively, liposome encapsulation of the protein antigen may be used to induce fusion with APC membrane and enhance uptake. Alternatively, uptake may be enhanced by adding one or more binding motifs that are recognized by receptors present on the surface of APCs. It is also possible to add a motif that is recognized by antibodies, which then interact with Fc receptors on APCs (Celis E. et al. Proc Natl Acad Sci U S A, 81: 6846-6850 (1984), entirely incorporated by reference).

Reducing APC Uptake

In a preferred embodiment, the parent protein is modified to reduce uptake by APCs. This may be accomplished by improving solubility or by modifying one or more sites on the protein that are recognized by receptors present on the surface of the APC.

Computational protein design approaches for improving the solubility of proteins have been described previously; see for example U.S. Ser. No. 10/338785, filed Jan. 6, 2003; 10/611,363, filed Jul. 3, 2003; U.S. Ser. No. 10/676,705, filed Sep. 30, 2003; PCT US/03100393, filed Jan. 6, 2003; and PCT US/03/30802, filed Sep. 30, 2003, all entirely incorporated by reference.

Methods for sterically blocking interactions between protein therapeutics and APC cell-surface receptors have also been disclosed previously, see 60/456094, filed Mar. 20, 2003, entirely incorporated by reference.

Altering Antigen Processing

In a preferred embodiment, specific cleavage motifs for antigen processing and presentation are added or removed to increase the availability of one or more MHC agretopes for MHC binding. For example, it may be possible to decrease immunogenicity by adding a cleavage site within an immunogenic 9-mer peptide, since proteolysis of the 9-mer will substantially limit its ability to bind MHC molecules. As described herein, a number of methods may be used to identify cleavage sites for proteases in the class I or class II pathways.

Incorporating New Class I MHC Agretopes

In a preferred embodiment, potential MHC class I agretopes are added to a target protein as a means of inducing cellular immunity. Suitable sequences may be identified using any of the methods described herein for the identification of class I MHC agretopes; sequences that are predicted to have enhanced binding affinity for one or more alleles may confer increased immunogenicity. Preferably at least one MHC class I binding site is added per target protein. More preferably at least 2 MHC class I binding sites are added per target protein. More preferably between 3 to 5 MHC class I binding sites are added per target protein. In other embodiments, up to 16 MHC class I binding sites may be added per target protein (see Stienekemeier, M., et al., (2001) Proc Natl Acad Sci USA, 98:13872-13877, entirely incorporated by reference).

New MHC agretopes can be incorporated into the parent protein in any region. In a preferred embodiment, the location of the new agretope is selected to minimize the number of mutations that must be introduced in order to confer the desired increase in immunogenicity. In an alternate preferred embodiment, the location of the new agretope is selected to minimize structural disruption. For example, the new agretope may be incorporated at the N- or C-terminus or within a loop region.

In one embodiment, for one or more sites of class I agretope addition identified above, one or more possible alternate 8-mer or 9-mer sequences is analyzed for immunogenicity. A preferred alternate sequences are then defined as those sequences that have high predicted immunogenicity. In a preferred embodiment, more immunogenic variants of each agretope exhibit increased binding affinity for at least one class I MHC allele. In an especially preferred embodiment, the more immunogenic variant of each agretope is predicted to bind to MHC alleles that are present in more than 10% of the relevant subject population, with more than 25% or 50% being more preferred.

Removing Class I MHC Agretopes

In a preferred embodiment, potential MHC class I binding sites are modified to reduce or eliminate peptide binding to MHC class I molecules. This may be accomplished by modifying the anchor residues or the non-anchor residues. Suitable sequences may be identified using any of the methods described herein for the identification of class I MHC agretopes; sequences that are predicted to have reduced binding affinity for one or more alleles may confer reduced immunogenicity.

In one embodiment, for one or more class I agretopes identified above, one or more possible alternate 8-mer or 9-mer sequences is analyzed for immunogenicity. A preferred alternate sequences are then defined as those sequences that have low predicted immunogenicity. In a preferred embodiment, less immunogenic variants of each agretope exhibit reduced binding affinity for at least one class I MHC allele. In an especially preferred embodiment, the less immunogenic variant of each agretope is predicted to bind to MHC alleles that are present in not more than 10% of the relevant subject population, with not more than 1% or 0.1% being more preferred.

Incorporating Class II MHC Agretopes

In a preferred embodiment, potential MHC class II agretopes are added to a target protein as a means of inducing humoral immunity. Suitable sequences may be identified using any of the methods described herein for the identification of class II MHC agretopes; sequences that are predicted to have enhanced binding affinity for one or more alleles may confer increased immunogenicity. Preferably at least one MHC class II binding site is added per target protein. More preferably at least 2 MHC class II binding sites are added per target protein. More preferably between 3 to 5 MHC class II binding sites are added per target protein. In other embodiments, up to 16 MHC class I binding sites may be added per target protein (see Stienekemeier, M., et al., (supra)).

New MHC agretopes can be incorporated into the parent protein in any region. In a preferred embodiment, the location of the new agretope is selected to minimize the number of mutations that must be introduced in order to confer the desired increase in immunogenicity. In an alternate preferred embodiment, the location of the new agretope is selected to minimize structural disruption. For example, the new agretope may be incorporated at the N- or C-terminus or within a loop region.

In one embodiment, for one or more sites of class I agretope addition identified above, one or more possible alternate 8-mer or 9-mer sequences is analyzed for immunogenicity. A preferred alternate sequences are then defined as those sequences that have high predicted immunogenicity. In a preferred embodiment, more immunogenic variants of each agretope exhibit increased binding affinity for at least one class II MHC allele. In an especially preferred embodiment, the more immunogenic variant of each agretope is predicted to bind to MHC alleles that are present in more than 10% of the relevant subject population, with more than 25% or 50% being more preferred.

Removing Class II MHC Agretopes

In a preferred embodiment, one or more of the above-determined class II MHC-binding agretopes are replaced with alternate amino acid sequences to generate variant proteins with reduced immunogenicity. Either anchoring residues, non-anchoring residues, or both may be replaced.

In one embodiment, for one or more class II agretopes identified above, one or more possible alternate 9-mer sequences is analyzed for immunogenicity. A preferred alternate sequences are then defined as those sequences that have low predicted immunogenicity. In a preferred embodiment, less immunogenic variants of each agretope exhibit reduced binding affinity for at least one class II MHC allele. In an especially preferred embodiment, the less immunogenic variant of each agretope is predicted to bind to MHC alleles that are present in not more than 10% of the relevant subject population, with not more than 1% or 0.1% being more preferred.

The fundamental binding units of class I and class II MHC proteins are both 9 amino acids. However, it is also known that additional peptide flanking residues (PFRs) can influence T-cell recognition (via the TCR) of class II MHC-peptide complexes (see for example Arnold et al., 2002, J Immunology 169(2):739-49, entirely incorporated by reference), with the residues at positions P-1 (one position before the 1^{st} MHC binding position) and P11 being most influential. In an alternative embodiment, residues at these positions can be engineered to reduce immunogenicity of a therapeutic protein.

Incorporating T Cell Epitope Antagonists

In a preferred embodiment, synthetic amino acids or amino acid analogs are incorporated to generate MHC class I or class II ligands with antagonistic properties. Such peptides may be recognized by T cells, but instead of eliciting an immune response, act to block immune responses to the cognate epitope. Generally, antagonists are derived from known epitopes by amino acid replacements that introduce charge or bulky size modification of peptide side chains. Preferably, N-hydroxylated peptide derivatives, or beta-amino acids are introduced into T cell epitopes to generate antagonists (see for example, Hin, S., et al., (1999) J. Immunology, 163:2363-2367; Reinelt, S., et al., (2001) J. Biol. Chem., 276:24525-24530), both entirely incorporated by reference.

Removing Antibody Epitopes

Rules for determining suitable replacements of antibody binding surface residues are emerging (see Meyer, D. L., et al. (2001) Protein Science, 10:491-503; Laroche, Y., (2000) Blood, 96:1425-1432; and Schwartz, H. L., (1999) J. Mol. Biol., 287:983-999, all entirely incorporated by reference). For example, aromatic surface residues such as tyrosine are often implicated in antigen-antibody binding. In a preferred embodiment, aromatic and charged residues in an antibody epitope may be replaced with smaller neutral residues, such as serine, threonine, asparagine, alanine or glycine.

Sterically Blocking Antibody Binding

Covalent derivatization of the parent protein, for example PEGylation or other chemical modification, may be used to sterically interfere with antibody binding. In a preferred embodiment, the site of PEG addition is selected to be within 10 Å of at least one residue in an antibody epitope, with less than 5 Å being especially preferred. Furthermore, the size and branching structure of the PEG molecule may be selected to most effectively interfere with antibody binding. For example, branched PEG molecules may be more effective for immunogenicity reduction than linear PEG molecules of the same molecular weight (Caliceti and Veronese, Adv. Drug. Deliv. Rev. 55: 1261-1277 (2003), entirely incorporated by reference).

Identifying Variants with Desired Functional Properties

Modifications, such as those introduced to modulate immunogenicity, may negatively impact function in a number of ways. Mutations may directly reduce function, for example by reducing receptor binding affinity. Mutations may also reduce function indirectly by reducing the stability or solubility of the protein. Similarly, mutations may alter bioavailability. Modifications such as PEGylation may also reduce function by interfering with the formation of desired intermolecular interactions. Accordingly, in a preferred embodiment, protein stability and solubility are considered in the course of identifying variants with desired functional properties.

Two basic strategies may be used to identify variants that are likely to possess desired functional properties. If sufficient biochemical and structural data is available to directly model relevant functional properties of the parent protein and the variant proteins. For example, if binding with high affinity to a particular receptor is a desired function, energy calculations may be performed on the complex structure in order to determine whether the variant protein has decreased binding affinity. More commonly, modifications interfere with protein function by destabilizing the protein structure. Accordingly, in a preferred embodiment, the variant protein is computationally analyzed to determine whether it is likely to assume substantially the same structure as the target protein and whether the variant protein is likely to retain sufficient stability to perform the desired functions.

Structure-Based Methods

In a preferred embodiment, structure based methods are used to identify variant sequences that are capable of stably assuming a structure that is substantially similar to the structure of the parent protein. In addition, it is preferred that structure based methods are also used to identify variant sequences that retain binding affinity for desired molecules.

In a preferred embodiment, the computational method used to identify protein sequences with desired functional properties is Protein Design Automation® (PDA®) technology, as is described in U.S. Pat. Nos. 6,188,965; 6,269,312; 6,403,312; WO98/47089 and U.S. Ser. Nos. 09/058,459, 09/714,357, 09/812,034, 09/827,960, 09/837,886, 09/877,695, 10/071,85909/419,351, 09/782,004 and 09/927,790, 60/347,772, 10/101,499, and 10/218,102; and PCT/US01/218,102 and U.S. Ser. No. 10/218,102, U.S. Ser. No. 60/345,805; U.S. Ser. No. 60/373,453 and U.S. Ser. No. 60/374,035, all entirely incorporated by reference. Briefly, PDA® technology may be described as follows. A protein structure (which may be determined experimentally, generated by homology modeling or produced de novo) is used as the starting point. The positions that are allowed to vary are then identified, which may be the entire sequence or subset(s) thereof. The amino acids that are considered at each variable position are selected. Optionally, each amino acid residue may be represented by a discrete set of allowed conformations, called rotamers. Interaction energies are calculated using a scoring function between (1) each allowed residue or rotamer at each variable position and the backbone, (2) each allowed residue or rotamer at each variable position and each non-variable residue (if any), and (3) each allowed residue or rotamer at each variable position and each allowed residue or rotamer at each other variable position. Combinatorial search algorithms, typically DEE and Monte Carlo, are used to identify the optimum amino acid sequence and additional low energy sequences. The resulting sequences may be generated experimentally or subjected to further computational analysis.

Especially favored structure-based methods calculate scores or energies that report the suitability of different variant protein sequences for a target protein structure. In many cases, these methods enable the computational screening of a very large number of variant protein sequences and variant protein structures (in cases where different side chain conformations are explicitly considered). See, for example, (Dahiyat and Mayo, Protein Sci 5(5): 895-903 (1996); Dahiyat and Mayo, Science 278(5335): 82-7 (1997); Desjarlais and Handel, Protein Science 4: 2006-2018 (1995); Harbury et al, PNAS USA 92(18): 8408-8412 (1995); Kono et al., Proteins: Structure, Function and Genetics 19: 244-255 (1994); Hellinga and Richards, PNAS USA 91: 5803-5807 (1994), all entirely incorporated by reference). It is also possible to use statistical methods, including but not limited to those that assess the suitability of different amino acid residues for specific structural contexts (Bowie and Eisenberg, Science 253(5016): 164-70, (1991), entirely incorporated by reference), or “residue pair potentials” that score pairs of interacting residues based on the frequency of similar interactions in proteins of known structure (Miyazawa et al., Macromolecules 18(3): 534-552 (1985) Jones, Protein Sci 3: 567-574, (1994); PROSA (Heindlich et al., J. Mol. Biol. 216:167-180 (1990); THREADER (Jones et al., Nature 358:86-89 (1992), all entirely incorporated by reference.

In an especially preferred embodiment, Protein Design Automation® (PDA®) technology is used to identify variant proteins with desired functional properties. (See U.S. Pat. Nos. 6,188,965; 6,269,312; 6,403,312; WO98/47089 and U.S. Ser. Nos. 09/058,459, 09/714,357, 09/812,034, 09/827,960, 09/837,886, 09/877,695, 10/071,85909/419,351, 09/782,004 and 09/927,790, 60/347,772, 10/101,499, and 10/218,102; and PCT/US01/218,102 and U.S. Ser. No. 10/218,102, U.S. Ser. No. 60/345,805; U.S. Ser. No. 60/373,453 and U.S. Ser. No. 60/374,035, all entirely incorporated by reference). PDA® calculations may be used to identify protein sequences that are likely to be stable and adopt a given fold. In addition, PDA® calculations may be used to predict the binding affinity of a given protein for one or more binding partners, including but not limited to other proteins, sugars, small molecules, or nucleic acids.

In a preferred embodiment, the PDA® energy of the variant protein is increased by no more than 10% relative to the parent protein, with equal energies or more favorable energies being especially preferred. Similarly, if PDA® calculations are performed to determine the affinity of an intermolecular interaction, it is preferred that the interaction energy for the variant protein is increased by no more than 10%, and equal energies or more favorable energies are especially preferred.

Sequence-Based Methods

In an alternate embodiment, substitution matrices or other knowledge-based scoring methods are used to identify alternate sequences that are likely to retain the structure and function of the wild type protein. The substitution matrices may be general protein substitution matrices such as PAM or BLOSUM, or may be derived for a given protein family of interest. Such scoring methods can be used to quantify how conservative a given substitution or set of substitutions is. In most cases, conservative mutations do not significantly disrupt the structure and function of proteins (see for example, Bowie et al. Science 247: 1306-1310 (1990), Bowie and Sauer, Proc. Nat. Acad. Sci. USA 86: 2152-2156 (1989), and Reidhaar-Olson and Sauer Proteins 7: 306-316 (1990), all entirely incorporated by reference). However, non-conservative mutations can destabilize protein structure and reduce activity (see for example, Lim et. al. Biochem. 31: 4324-4333 (1992), entirely incorporated by reference). Substitution matrices provide a quantitative measure of the compatibility between a sequence and a target structure, which can be used to predict non-disruptive substitution mutations (see Topham et al. Prot. Eng. 10: 7-21 (1997), entirely incorporated by reference). The use of substitution matrices to design peptides with improved properties has been disclosed; see Adenot et al. J. Mol. Graph. Model. 17: 292-309 (1999), entirely incorporated by reference.

In a preferred embodiment, substitution mutations are preferentially introduced at positions that are substantially solvent exposed. As is known in the art, solvent exposed positions are typically more tolerant of mutation than positions that are located in the core of the protein.

In a preferred embodiment, substitution mutations are preferentially introduced at positions that are not highly conserved. As is known in the art, positions that are highly conserved among members of a protein family are often important for protein function, stability, or structure, while positions that are not highly conserved often can be modified without significantly impacting the structural or functional properties of the protein.

Identifying Compensatory Mutations

One special application of computational protein design algorithms is the identification of additional mutations that compensate for modifications that were introduced to modulate immunogenicity. For example, a mutation that greatly reduces immunogenicity may be destabilizing to the protein structure. Computational protein design methods may be used to identify additional mutations that will stabilize the protein. Similarly, if a modification made to reduce immunogenicity reduces receptor binding affinity, computational protein design methods may be used to identify mutations that confer increased receptor binding affinity.

Identifying Variants with Desired Immunological and Functional Properties

Immunogenicity considerations may be directly incorporated into computational protein design algorithms in any of a number of ways. It is possible to combine two or more of these methods, if desired.

Selection of Residue Choices for each Variable Position

In one embodiment, immunogenicity considerations are used to influence the set of amino acids that are allowed at each variable position. For example, large hydrophobic residues may be excluded at solvent exposed positions to prevent the creation of a new antibody epitope or MHC agretope. Similarly, if a given substitution will increase binding to one or more MHC alleles, regardless of the residues selected at the other variable positions, it may be eliminated from consideration. It is also possible to restrict residue choices to the set of residues that can act as PEG attachment sites.

Incorporating MHC Binding Affinity into Monte Carlo Calculations

In an alternate embodiment, MHC binding propensities are incorporated during a Monte Carlo calculation. Monte Carlo calculations are often performed during the course of protein design calculations in order to identify one or more sequences that have favorable energies or scores. The calculation may be modified by assessing the number and strength of predicted MHC agretopes in each sequence, and favoring steps that decrease (or increase, if immunogenicity enhancement is the goal) the predicted number or strength of the MHC agretopes.

Incorporating MHC Binding Affinity into Dead-End Elimination Calculations

In an alternate embodiment, MHC binding propensities are incorporated during a DEE calculation. DEE calculations are often performed during the course of protein design calculations in order to identify the variant sequence that has the most favorable energy or score. Typically, DEE requires energy terms that are pair-wise decomposable, meaning that they depend on the identity of two residues only. Properties such as MHC binding affinity that depend on the identity of three or more residues may be incorporated into DEE during the “Unification” step. The “Unification” step combines two rotamers into one “super-rotamer”, and eliminates super-rotamers with unfavorable scores or energies. Similarly, super-rotamers comprising one or more MHC agretopes may be eliminated.

Incorporating MHC Binding Affinity into Branch and Bound Calculations

In an alternate embodiment, MHC binding propensities are incorporated during a Branch and Bound calculation. Branch and Bound calculations are often performed during the course of protein design calculations in order to identify one or more sequences that have favorable energies or scores. Potential sequences are constructed one residue at a time. If it can be demonstrated that all sequences comprising a given partial sequence have energies or scores that are worse than some cutoff value, a “bound” is placed on that partial sequence and it is not considered further. Similarly, if it can be demonstrated that all sequences comprising a given partial sequence comprise immunogenic MHC agretopes, the partial sequence may be bound.

Additional Modifications

Additional insertions, deletions, and substitutions may be incorporated into the variant proteins of the invention in order to confer other desired properties.

In one embodiment, additional modifications are introduced to alter properties such as stability, solubility, and receptor binding affinity. Such modifications can also contribute to immunogenicity reduction. For example, since protein aggregates have been observed to be more immunogenic than soluble proteins, modifications that improve solubility may reduce immunogenicity (see for example Braun et. al. Pharm. Res. 14: 1472 (1997) and Speidel et. al. Eur. J. Immunol. 27: 2391 (1997), both entirely incorporated by reference).

Glycosylation

In one embodiment, the sequence of the variant protein is modified in order to add or remove one or more N-linked or O-linked glycosylation sites. Addition of glycosylation sites to variant proteins may be accomplished by the incorporation of one or more serine or threonine residues to the native sequence or variant protein (for O-linked glycosylation sites) or by the incorporation of a canonical N-linked glycosylation site, including but not limited to, N—X—Y, where X is any amino acid except for proline and Y is preferably threonine, serine or cysteine. Glycosylation sites may be removed by replacing one or more serine or threonine residues or by replacing one or more canonical N-linked glycosylation sites.

In another preferred embodiment, cysteines or other reactive amino acids are designed into the variant proteins in order to incorporate labeling sites or PEGylation sites.

Cyclization and Circular Permutation

In another preferred embodiment, the N- and C-termini of a variant protein are joined to create a cyclized or circularly permutated protein. Various techniques may be used to permutate proteins. See U.S. Pat. No. 5,981,200; Maki K, Iwakura M., Seikagaku. January 2001;73(1): 42-6; Pan T., Methods Enzymol. 2000; 317:313-30; Heinemann U, Hahn M., Prog Biophys Mol Biol. 1995; 64(2-3): 121-43; Harris M E, Pace N R, Mol Biol Rep. 1995-96; 22(2-3): 115-23; Pan T, Uhlenbeck O C., Mar. 30, 1993; 125(2): 111-4; Nardulli A M, Shapiro D J. 1993 Winter; 3(4):247-55, EP 1098257 A2; WO 02/22149; WO 01/51629; WO 99/51632; Hennecke, et al., 1999, J. Mol. Biol., 286, 1197-1215; Goldenberg et al J. Mol. Biol 165, 407-413 (1983); Luger et al, Science, 243, 206-210 (1989); and Zhang et al., Protein Sci 5, 1290-1300 (1996); all entirely incorporated by reference.

To produce a circularly permuted variant protein, a novel set of N- and C-termini are created at amino acid positions normally internal to the protein's primary structure, and the original N- and C-termini are joined via a peptide linker consisting of from 0 to 30 amino acids in length (in some cases, some of the amino acids located near the original termini are removed to accommodate the linker design). In a preferred embodiment, the novel N- and C-termini are located in a non-regular secondary structural element, such as a loop or turn, such that the stability and activity of the novel protein are similar to those of the original protein. The circularly permuted variant protein may be further PEGylated or glycosylated. In a further preferred embodiment PDA® technology may be used to further optimize the variant protein, particularly in the regions created by circular permutation. These include the novel N- and C-termini, as well as the original termini and linker peptide.

In addition, a completely cyclic variant protein may be generated, wherein the protein contains no termini. This is accomplished utilizing intein technology. Thus, peptides can be cyclized and in particular inteins may be utilized to accomplish the cyclization.

Tags and Fusion Constructs

Variant proteins of the present invention may also be modified to form chimeric molecules comprising a variant protein fused to another, heterologous polypeptide or amino acid sequence.

Variant proteins of the present invention may also be fused to another, heterologous polypeptide or amino acid sequence to form a chimera. The chimeric molecule may comprise a fusion of a variant protein with an immunoglobulin or a particular region of an immunoglobulin such as the Fc or Fab regions of an IgG molecule. In another embodiment, the variant protein is fused with human serum albumin to improve pharmacokinetics.

In an alternative embodiment, the chimeric molecule comprises a variant protein and a tag polypeptide which provides an epitope to which an anti-tag antibody can selectively bind. The epitope tag is generally placed at the amino-or carboxyl-terminus of the variant protein. The presence of such epitope-tagged forms of a variant protein can be detected using an antibody against the tag polypeptide. Also, provision of the epitope tag enables the variant protein to be readily purified by affinity purification using an anti-tag antibody or another type of affinity matrix that binds to the epitope tag. Various tag polypeptides and their respective antibodies are well known in the art. Examples include poly-histidine (poly-His) or poly-histidine-glycine (poly-His-Gly) tags; the flu HA tag polypeptide and its antibody 12CA5 (Field et al., Mol. Cell. Biol. 8:2159-2165 (1988)); the c-myc tag and the 8F9, 3C7, 6E10, G4, B7 and 9E10 antibodies thereto (Evan et al., Molecular and Cellular Biology, 5:3610-3616 (1985)); and the Herpes Simplex virus glycoprotein D (gD) tag and its antibody (Paborsky et al., Protein Engineering, 3(6): 547-553 (1990), all entirely incorporated by reference). Other tag polypeptides include the Flag-peptide (Hopp et al., BioTechnology 6:1204-1210 (1988)); the KT3 epitope peptide (Martin et al., Science 255:192-194 (1992)); tubulin epitope peptide (Skinner et al., J. Biol. Chem. 266:15163-15166 (1991)); and the T7 gene 10 protein peptide tag (Lutz-Freyermuth et al., Proc. Natl. Acad. Sci. U.S.A. 87:6393-6397 (1990), all entirely incorporated by reference).

Generating Variants

Variant proteins of the invention and nucleic acids encoding them may be produced using a number of methods known in the art, as illustrated herein.

Generating Nucleic Acid Encoding the Variant Protein

In a preferred embodiment, nucleic acids encoding the variant proteins are prepared by total gene synthesis or by site-directed mutagenesis of a nucleic acid encoding a parent protein. Methods including template-directed ligation, recursive PCR, cassette mutagenesis, site-directed mutagenesis or other techniques that are well known in the art may be utilized (see for example Strizhov et al. PNAS 93:15012-15017 (1996), Prodromou and Perl, Prot. Eng. 5: 827-829 (1992), Jayaraman and Puccini, Biotechniques 12: 392-398 (1992), and Chalmers et al. Biotechniques 30: 249-252 (2001), all entirely incorporated by reference).

Protein Expression

Appropriate host cells for the expression of the variant proteins include yeast, bacteria, archaebacteria, fungi, and insect and animal cells, including mammalian cells. Of particular interest are bacteria such as E. coli and Bacillus subtilis, fungi such as Saccharomyces cerevisiae, Pichia pastoris, and Neurospora, insects such as Drosophila melangaster and insect cell lines such as SF9, mammalian cell lines including 293, CHO, COS, Jurkat, NIH3T3, etc. (see the ATCC cell line catalog). The variant proteins of the present invention may be produced by culturing a host cell transformed with an expression vector containing nucleic acid encoding a variant protein, under the appropriate conditions to induce or cause expression of the variant protein. The conditions appropriate for variant protein expression will vary with the choice of the expression vector and the host cell, and are easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

In a preferred embodiment, variant proteins are expressed in E. coli. Bacterial expression systems and methods for their use are well known in the art (see Current Protocols in Molecular Biology, Wiley & Sons, and Molecular Cloning—A Laboratory Manual—3rd Ed., Cold Spring Harbor Laboratory Press, New York (2001), entirely incorporated by reference). The choice of codons, suitable expression vectors and suitable host cells will vary depending on a number of factors, and may be easily optimized as needed. In an alternate preferred embodiment, variant proteins are expressed in mammalian cells or in other expression systems including but not limited to yeast, baculovirus, and in vitro expression systems.

In one embodiment, the variant nucleic acids, proteins and antibodies of the invention are labeled with a label other than the scaffold. By “labeled” herein is meant that a compound has at least one element, isotope or chemical compound attached to enable the detection of the compound. In general, labels fall into three classes: a) isotopic labels, which may be radioactive or heavy isotopes; b) immune labels, which may be antibodies or antigens; and c) colored or fluorescent dyes. The labels may be incorporated into the compound at any position.

Protein Purification

In a preferred embodiment, the variant proteins are purified or isolated after expression. Standard purification methods include electrophoretic, molecular, immunological and chromatographic techniques, including ion exchange, hydrophobic, affinity, and reverse-phase HPLC chromatography, and chromatofocusing. For example, a variant protein may be purified using a standard anti-recombinant protein antibody column. Ultrafiltration and diafiltration techniques, in conjunction with protein concentration, are also useful. For general guidance in suitable purification techniques, see Scopes, R., Protein Purification, Springer-Verlag, NY, 3rd ed. (1994), entirely incorporated by reference. The degree of purification necessary will vary depending on the desired use, and in some instances no purification is necessary.

Posttranslational Modification and Derivatization

Once made, the variant proteins may be covalently modified. Covalent and non-covalent modifications of the protein are thus included within the scope of the present invention. Such modifications may be introduced into a variant protein by reacting targeted amino acid residues of the protein with an organic derivatizing agent that is capable of reacting with selected side chains or terminal residues. Optimal sites for modification can be chosen using a variety of criteria, including but not limited to, visual inspection, structural analysis, sequence analysis, and molecular simulation.

In one embodiment, the variant proteins of the invention are labeled with at least one element, isotope or chemical compound. In general, labels fall into three classes: a) isotopic labels, which may be radioactive or heavy isotopes; b) immune labels, which may be antibodies or antigens; and c) colored or fluorescent dyes. The labels may be incorporated into the compound at any position. Labels include but are not limited to biotin, tag (e.g. FLAG, Myc) and fluorescent labels (e.g. fluorescein).

One type of covalent modification includes reacting targeted amino acid residues of a variant TPO polypeptide with an organic derivatizing agent that is capable of reacting with selected side chains or the N-or C-terminal residues of a variant protein. Derivatization with bifunctional agents is useful, for instance, for cross linking a variant protein to a water-insoluble support matrix or surface for use in the method for purifying anti-variant protein antibodies or screening assays, as is more fully described below. Commonly used cross linking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the amino groups of lysine, arginine, and histidine side chains (T. E. Creighton, Proteins: Structure and Molecular Properties, W.H. Freeman & Co., San Francisco, pp. 79-86 (1983), entirely incorporated by reference), acetylation of the N-terminal amine, and amidation of any C-terminal carboxyl group.

Such derivatization may improve the solubility, absorption, permeability across the blood brain barrier, serum half life, and the like. Modifications of variant proteins may alternatively eliminate or attenuate any possible undesirable side effect of the protein. Moieties capable of mediating such effects are disclosed, for example, in Remington's Pharmaceutical Sciences, 16th ed., Mack Publishing Co., Easton, Pa. (1980), entirely incorporated by reference.

Another type of covalent modification of variant proteins comprises linking the variant protein to one of a variety of nonproteinaceous polymers, e.g., polyethylene glycol (“PEG”), polypropylene glycol, or polyoxyalkylenes, in the manner set forth in U.S. Pat. Nos. 4,640,835; 4,496,689; 4,301,144; 4,670,417; 4,791,192 and 4,179,337, all entirely incorporated by reference. A variety of coupling chemistries may be used to achieve PEG attachment, as is well known in the art. Examples include but are not limited to, the technologies of Shearwater and Enzon, which allow modification at primary amines, including but not limited to, lysine groups and the N-terminus. See, Kinstler et al, Advanced Drug Deliveries Reviews, 54, 477-485 (2002) and M J Roberts et al, Advanced Drug Delivery Reviews, 54, 459-476 (2002), both entirely incorporated by reference. It is also possible to modify the variant proteins by covalently attaching a covalent polymer, for example as described in WO 01/41812A2, entirely incorporated by reference.

Assaying the Activity of the Variants

The variant proteins of the invention may be tested for activity using any of a number of methods, including but not limited to receptor binding assays, cell-based activity assays, and in vivo assays. Suitable assays will vary according to the identity of the parent protein and may easily be identified by one skilled in the art.

Assaying the Immunogenicity of the Variants

In a preferred embodiment, the immunogenicity of the variant proteins is determined experimentally to confirm that the variants do have enhanced or reduced immunogenicity, as desired, relative to the parent protein. Alternatively, the immunogenicity of a novel protein may be assessed.

Antigen Uptake Assays

Uptake of the variant proteins by APCs may be determined. There are a number of methods that can be used to assess the extent to which the variant protein is internalized within the APCs. For example, it is possible to fluorescently label the variant protein and use imaging methods to monitor uptake. It is also possible to fix APCs and stain them using a labeled antibody that recognizes the variant protein of interest (Inaba et al. J. Exp. Med. 188: 2163-2173 (1998), Mahnke et. al. J. Cell. Biol. 151: 673-683 (2000), both entirely incorporated by reference). It is also possible to measure disappearance from media containing the cells. In an especially preferred embodiment, the subcellular localization of the antigen is determined.

Protein antigen interaction with certain receptors (e.g., mannose receptor; Tan M C, Mommaas A M, Drijfhout J W, Jordens R, Onderwater J J, Verwoerd D, Mulder A A, van der Heiden A N, Ottenhoff T H, Celia M, Tulp A, Neefjes J J, Koning F. “Mannose receptor mediated uptake of antigens strongly enhances HLA-class II restricted antigen presentation by cultured dentritic cells” Adv Exp Med Biol, 417, 171-4 (1997); entirely incorporated by reference) on the surface of APC increases the efficiency of protein antigen uptake. The most common professional APC in humans, dendritic cells and macrophages, display surface Fc receptors, which specifically bind to the Fc portion of IgG. By coupling a protein tag and an antibody specific for that tag, antibody-mediated targeting (Celis E, Zurawski V R Jr, Chang T W. “Regulation of T cell function by antibodies: enhancement of the response of human T cell clones to hepatitis B surface antigen by antigen-specific monoclonal antibodies” Proc Natl Acad Sci U S A, 81, 6846-50 (1984), entirely incorporated by reference) of the APC may increase protein antigen uptake.

Alternatively, liposome encapsulation of protein antigen could induce fusion with APC membrane and enhance uptake.

MHC Binding Assays

In a preferred embodiment, the variant proteins are assayed for the presence of MHC agretopes. A number of methods may be used to measure peptide interactions with MHC, including but not limited to those described in a recent review (Fleckenstein et al. Sem. Immunol. 11: 405-416 (1999), entirely incorporated by reference) and those discussed below.

In one embodiment, the variant proteins may be screened for MHC binding using a series of overlapping peptides. It is possible to assay peptide-MHC binding in solution, for example by fluorescently labeling the peptide and monitoring fluorescence polarization (Dedier et al. J. Immuno. Meth. 255: 57-66 (2001), entirely incorporated by reference). It is also possible to use mass spectrometry methods (Lemmel and Stevanovic, Methods 29: 248-259 (2003), entirely incorporated by reference).

T Cell Activation Assays

In a preferred embodiment, ex vivo T cell activation assays are used to experimentally quantitate immunogenicity (see for example Fleckenstein supra, Schmittel et. al. J. Immunol. Meth., 24:17-24 (2000), Anthony and Lehmann Methods 29: 260-269 (2003), Stickler et al. J. Immunother. 23: 654-660 (2000), Hoffmeister et al. Methods 29: 270-281 (2003) and Schultes and Whiteside, J. Immunol. Meth. 279: 1-15 (2003), all entirely incorporated by reference). Any of a number of assay protocols can be used; these protocols differ regarding the mode of antigen presentation (MHC tetramers, intact APCs), the form of the antigen (peptide fragments or whole protein), the number of rounds of stimulation, and the method of detection (Elispot detection of cytokine production, flow cytometry, tritiated thymidine incorporation). Prior to this invention no clinically validated assay for testing immunogenicity has been described, although several protocols had been published to measure T cell activation ex vivo.

In a more preferred embodiment, APCs and CD4+ T cells from matched donors are challenged with a peptide or whole protein of interest as determined experimentally to mimic the in vivo conditions (as described herein), and T cell activation is monitored using Elispot assays for cytokine production.

In addition, suitable assays include those disclosed in Meidenbauer, N., Harris, D. T., Spitler, L. E., Whiteside, T. L., 2000, entirely incorporated by reference. Generation of PSA-reactive effector cells after vaccination with a PSA-based vaccine in subjects with prostate cancer. Prostate 43, 88-100 and Schultes, B. C and Whiteside, T. L., 2003. Monitoring of Immune Responses to CA125 with an IFN-γ ELISPOT Assay. J. Immunol. Methods 279, 1-15, both entirely incorporated by reference.

There are different ways to prime the T cells ex vivo. The antigen presenting cells (APCs) may be loaded with individual peptides, and selected T cells tested with the same peptides. In a preferred embodiment, the T cells can be primed with a combination of several peptides, and then tested with individual ones. In a preferred embodiment, the T cells can be selected with multiple rounds of stimulation with APCs loaded with proteins, and then tested with individual peptides from that protein to identify physiologically relevant epitopes. The conditions selected are the ones that better mimic the steps in vivo, as determined with blood from immunized subjects.

Delineating potential immunogenic T cell epitopes within intact proteins is usually carried out by making overlapping synthetic peptides spanning the protein's sequence and using these peptides in T cell proliferation assays (see Stickler, M M, Estell, D A, Harding, F A “CD4+ T-Cell Epitope Determination Using Unexposed Human Donor Peripheral Blood Mononuclear Cells” J. Immunotherapy, 23, 654-660 (2000), entirely incorporated by reference). Uptake of peptides for MHC presentation by the APC is not required since sufficient empty MHC class II molecules generally exist on the surface of most APC and bind sufficient quantity of peptide. While uptake and presentation of antigens derived from intact protein in these in vitro assays can be less efficient in the absence of receptor-mediated endocytosis, the use of intact protein is beneficial because the use of intact proteins will more closely mimic the physiological antigen processing pathway, thereby reducing the number of false immunogenic positives.

In a preferred embodiment of an IVV T cell assay, a DNA construct is made that includes attaching a tag (e.g, Myc, His, S-tag, Flag) to the protein. A preferred tag should itself be non-immunogenic and will have commercially available mouse monoclonal antibodies. In addition, a humanized anti-tag antibody is used. The humanized anti-tag antibody is generated preferably by grafting the mouse variable regions onto a human IgG scaffold or by removing T-helper cell epitopes. The protein-tag-antibody complex is introduced into a CD4(+) T cell assay in which the complex will target an antigen presenting cell (APC: e.g., dendritic cell or macrophage) via cell surface Fcγ receptors.

Protein antigen interaction with certain receptors (e.g., mannose receptor; Tan M C, Mommaas A M, Drijfhout J W, Jordens R, Onderwater J J, Verwoerd D, Mulder A A, van der Heiden A N, Ottenhoff T H, Cella M, Tulp A, Neefjes J J, Koning F. “Mannose receptor mediated uptake of antigens strongly enhances HLA-class II restricted antigen presentation by cultured dentritic cells” Adv Exp Med Biol, 417, 171-4 (1997); incorporated by reference) on the surface of APC increases the efficiency of protein antigen uptake. The most common professional APC in humans, dendritic cells and macrophages, display surface Fc receptors, which specifically bind to the Fc portion of IgG. By coupling a protein tag and an antibody specific for that tag, antibody-mediated targeting (Celis E, Zurawski V R Jr, Chang T W. “Regulation of T cell function by antibodies: enhancement of the response of human T cell clones to hepatitis B surface antigen by antigen-specific monoclonal antibodies” Proc Natl Acad Sci U S A, 81, 6846-50 (1984), entirely incorporated by reference) of the APC may increase protein antigen uptake. Alternatively, liposome encapsulation of protein antigen could induce fusion with APC membrane and enhance uptake.

In another preferred embodiment, reactive polyclonal T cell populations expanded with re-stimulation in the presence of MHC-restricted antigen are used to map the immunodominant epitopes present within the protein of interest.

A preferred assay may be performed using the following steps: (1) therapeutic agent is introduced to the antigen presenting cell (APC) and appropriate conditions found to stimulate efficient uptake and processing, (2) the APC with multiple MHC-restricted epitopes will stimulate initially naive T cells, (3) T cell re-stimulation will take place to ensure a large population of reactive polyclonal T cells, (4) this pool of reactive T cells are divided into smaller amounts, 5) potential peptide epitopes from the full length protein are synthesized based on either prediction or from an overlapping peptide library, 6) each peptide is tested for T cell reactivity for the samples from step (4) above. The testing may use, for example, the EliSPOT method.

The present invention provides ex vivo testing of T cell activation by endogenous or foreign proteins or peptides. CD4+ T cells are activated ex vivo by exposure to the antigen presenting cells loaded with whole proteins or peptides. T cells undergo negative selection during their development to minimize the number that are reactive to self-antigens. Hence, the vast majority of naïve T cells may not be reactive to many therapeutic proteins of human origin, and ex vivo immunogenicity testing in that capacity with naïve T cells may hinder the discovery of potential MHC-binding epitopes. Conditions for ex vivo activation of T cells that allow multiple rounds of selection are a preferred embodiment as it allows for further optimization. In a preferred embodiment, either peptides or whole proteins may be tested.

In a preferred embodiment, it is desirable to increase the population of reactive CD4+ T cells prior to the activation assay. As is known in the art, dendritic cells may be produced from proliferating dendritic cell precursors (See for example, USSN 2002/0085993, U.S. Pat. Nos. 5,994,126; 6,274,378; 5,851,756; and WO93/20185, hereby expressly incorporated by reference.). Dendritic cells pulsed with proteins or peptides are co-cultured with CD4+ T cells. T cell proliferation in the presence of antigen presenting dendritic cells simulate in vivo clonal expansion. EVV may be used for either whole proteins or peptides. The conditions of the assay are calibrated using conditions that reproduce the results observed with cells primed in vivo from subjects that developed neutralizing antibodies.

In a preferred embodiment, full length and truncated (receptor-binding domain) proteins may be tested with a preferred assay. Peptides derived from the protein sequence will also be evaluated, and the necessary number of exposures (dendritic cells vs. T cells) to obtain sufficient and measurable T cell activation determined. The proteins/peptides is tested with cells from several different donors (different alleles). Preferably, APCs are dendritic cells isolated either directly from subject PBMC or differentiated from subject monocytes. Antigen-dependent activation of CD4+ T-helper cells is required prior to the sustained production of the antibody isotype most relevant.

Enzymatic processing of exogenous antigens by professional antigen presenting cells (APC) provides a pool of potentially antigenic peptides from which proteins encoded in the Major Histocompatibility Complex (MHC class II molecules) are drawn from for loading and presentation to CD4+ T cells. T cells expressing the appropriate T cell receptor with basal affinity for the MHC/peptide complex on the APC surface activate and proliferate in response to the interaction. T cells isolated from “unprimed” individuals that have had little or no prior exposure to a particular antigen are said to be “naive”. During the development of T cells, positive and negative selection may take place. Positive selection ensures that the individual's T cell population expresses viable T cell receptors while negative selection minimizes the number of high affinity self-reactive T cells.

In Vivo Assays

In an alternate preferred embodiment, immunogenicity is measured in transgenic mouse systems. For example, mice expressing fully or partially human class II MHC molecules may be used (see for example Stewart et. al. Mol. Biol. Med. 6: 275-281 (1989), Sonderstrup et. al. Immunol. Rev. 172: 335-343 (1999) and Forsthuber et al. J. Immunol. 167: 119-125 (2001), both entirely incorporated by reference).

In another embodiment, immunogenicity is measured using mice reconstituted with human antigen-presenting cells and T cells in place of their endogenous cells (WO 98/52976; WO 00/34317, both entirely incorporated by reference).

In an alternate embodiment, immunogenicity is tested by administering the variant proteins of the invention to one or more animals, including rodents and primates, and monitoring for antibody formation. Non-human primates with defined MHC haplotypes may be especially useful, as the sequences and hence peptide binding specificities of the MHC molecules in non-human primates may be very similar to the sequences and peptide binding specificities of humans.

Formulation and Administration

Once made, the variant proteins and nucleic acids of the invention find use in a number of applications. In a preferred embodiment, the variant proteins are administered to a subject to prevent or treat a disease or disorder. Suitable diseases or disorders will vary according to the nature of the parent protein and may be determined by one skilled in the art. Administration may be therapeutic or prophylactic.

Formulation

The pharmaceutical compositions of the present invention comprise a variant protein in a form suitable for administration to a subject. In a preferred embodiment, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers such as NaOAc; fillers such as microcrystalline cellulose, lactose, corn and other starches; binding agents; sweeteners and other flavoring agents; coloring agents; and polyethylene glycol. Additives are well known in the art, and are used in a variety of formulations.

Administration of a Protein Therapeutic Using Standard Approaches

The administration of the variant proteins of the present invention, preferably in the form of a sterile aqueous solution, may be done in a variety of ways, including, but not limited to, orally, subcutaneously, intravenously, intranasally, transdermally, intraperitoneally, intramuscularly, parenterally, intrapulmonary, vaginally, rectally, or intraocularly. In some instances, for example, the variant protein may be directly applied as a solution or spray. Depending upon the manner of introduction, the pharmaceutical composition may be formulated in a variety of ways. In a preferred embodiment, a therapeutically effective dose of a variant protein is administered to a subject in need of treatment. By “therapeutically effective dose” herein is meant a dose that produces the effects for which it is administered. The exact dose will depend on the purpose of the treatment, and is ascertainable by one skilled in the art using known techniques. In a preferred embodiment, the concentration of the therapeutically active variant protein in the formulation may vary from about 0.1 to about 100 weight percent. In another preferred embodiment, the concentration of the variant protein is in the range of 0.003 to 1.0 molar. As is known in the art, adjustments for protein degradation, systemic versus localized delivery, and rate of new protease synthesis, as well as the age, body weight, general health, sex, diet, time of administration, drug interaction and the severity of the condition may be necessary, and is ascertainable with routine experimentation by those skilled in the art.

Combinations of pharmaceutical compositions may be administered. Moreover, the compositions may be administered in combination with other therapeutics.

Administration of a Protein Therapeutic Using Gene Therapy Approaches

In an alternate embodiment, nucleic acids encoding a variant protein may be administered; i.e., “gene therapy” approaches may be used. In this embodiment, variant nucleic acids are introduced into cells in a subject in order to achieve in vivo synthesis of a therapeutically effective amount of variant protein. Variant nucleic acids may be introduced using a number of techniques, including but not limited to transfection with liposomes, viral (typically retroviral) vectors, and viral coat protein-liposome mediated transfection (Dzau et al., Trends in Biotechnology 11:205-210 (1993), entirely incorporated by reference). In some situations, it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described (Wu et al., J. Biol. Chem. 262:4429-4432 (1987) and Wagner et al., Proc. Natl. Acad. Sci. U.S.A. 87:3410-3414 (1990), both entirely incorporated by reference). For review of gene marking and gene therapy protocols see Anderson et al., Science 256:808-813 (1992), entirely incorporated by reference.

Vaccine Administration

In a preferred embodiment, a variant protein of the invention is administered as a vaccine. Formulations and methods of administration described herein for protein therapeutics may also be suitable for protein vaccines. It is also possible to administer variant nucleic acids of the invention as DNA vaccines, such that the variant nucleic acid provides expression of the variant protein. Naked DNA vaccines are generally known in the art (Brower, Nature Biotechnology, 16:1304-1305 (1998), entirely incorporated by reference). The variant nucleic acid used for DNA vaccines may encode all or part of the variant protein.

In a preferred embodiment, the vaccines comprise an adjuvant molecule. Such adjuvant molecules include any chemical entity that increases the immunogenic response to the variant polypeptide or the protein encoded by the DNA vaccine (e.g. cytokines, pharmaceutically acceptable excipients, polymers, organic molecules, etc.).

### EXAMPLES

The following non-limiting examples illustrate aspects of the present application. The examples do not import or further limit the present application.

### Example 1

A broad strategy is used to: (A) identify clinically relevant MHC-binding epitopes in a target protein; (B) based on clinical evidence develop an ex vivo assay predictive of peptide and/or protein immunogenicity to humans; and, (C) design and screen protein variants with reduced immunogenicity.

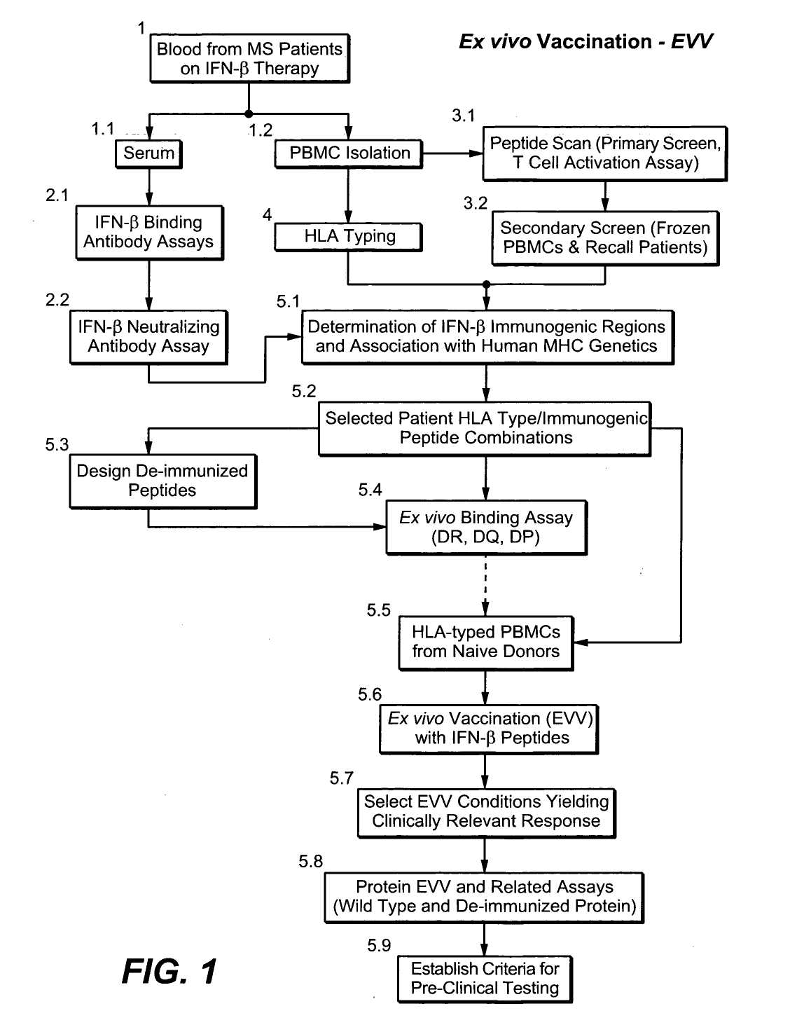

FIG. 1 shows a flowchart exemplifying the above-mentioned strategy (ex vivo vaccination—EVV) utilizing interferon beta (IFN-β) as the target protein. Other therapeutic proteins eliciting an immune response can also be utilized.

In the present example, blood from patients undergoing interferon beta therapy (10) was the source of serum (11) and peripheral blood mononuclear cells (PBMC) (12). Serum was tested regarding the presence of IFN-β binding antibodies (21) and neutralizing antibodies (22). This allowed identification of patients mounting an immune response to the drug (responders; positive antibody response) or not (non-responders; no antibody detected in the serum).

PBMC were used in an assay to map interferon beta epitopes (31). T-cell activation was tested with a set of overlapping peptides covering the entire IFN-β protein. (Pools of peptides with overlapping sequences can also be used.) A secondary screen was be used to confirm results and/or to identify the exact immunogenic region in a peptide pool (32). The secondary screen was performed either using frozen PBMC or cells recently collected from the suitable subject.

PBMC were further used as source of DNA for HLA typing (i.e., determining MHC composition,) and the genetic of responders and non-responders identified by PCR typing or other methods (41). This allowed investigation of a correlation between HLA type or alleles of the subjects and the development of antibodies (51).

Newly designed de-immunized peptides (53) can be tested ex vivo with a binding assay (54) and the peptides confirmed not to bind to MHC molecules of given donors tested with ex vivo vaccination, EVV. Selected patient HLA type/immunogenic peptide combinations (52) are used for the development of a clinically relevant assay that mimics the in vivo reactions (61 to 65). Typically responders and non-responders are included for the establishment of assay conditions, with the latter representing negative controls. The in vivo Cl response (i.e., clinical validation of the peptide) may be used to establish a benchmark for calibration of the ex vivo assays. HLA-typed PBMC containing naive T-cells are the source of cells for the assay.

When establishing conditions for EVV, a combination of assays can attain the desired results. Conditions used for the T cell activation assay should mimic the in vivo situation. One skilled in the art can also modify parts of this example to identify critical regions in a protein and to establish a link between factors leading to immunogenicity of a protein in vivo and the development of a clinically-relevant assay to access potential immunogenicity of a protein to mammals.

### Example2

Ex vivo assays supported the implementation of a strategy for clinical validation of the association between MHC-binding epitopes and interferon-beta immunogenicity. An optimized PBMC Elispot assay allowed sensitive detection of responsive T cells. The low detection limits for the assay permitted efficient epitope mapping using samples from patients that had developed or not developed antibodies to the drug. The assay was implemented and optimized using PBMC from normal donors and subsequently validated with samples from multiple sclerosis patients. IFN-β antibody binding assay (direct capture and indirect capture ELISA) allowed sensitive detection of IgG in serum from human subjects.

T Cell Activation Assay Using PBMC

The peripheral blood mononuclear cells (PBMC) are isolated from blood or lymphapheresis products using Ficoll for the separation (Amershan Biosciences,catalog number 17-1440-02) according to instructions from the manufacturer. Freshly isolated PBMC and/or cryo-preserved samples are used for the T cell activation assay.

The Elispot assay was performed with an Elispot kit to measure IFN-γ (Matbtech, catalog number 3420-2APW10), (it is also contemplated that the measurement could be made using other products allowing detection of IFN-γ secretion or other cytokine indicative of T cell activation). When using frozen PBMC the cells were quickly thawed and washed to remove any preservative agent such as DMSO that may be deleterious to the cells. The washes were performed with RPMI medium (Gibco catalog # 11875-093) or equivalent. Other buffered solutions that would equally protect the cells were also appropriate for the washes. Cells were suspended at a high cell concentration, adjusted according to the desired final cell number in each well of the Elispot plate. The medium used to suspend the cells and for the Elispot test (complete medium) can be RPMI supplemented with 5% human AB serum, 20 nM L-glutamine, 1.1 mg/ml sodium pyruvate, and non essential amino acids (for example Hyclone catalog number SH30238.01). The cytokines IL-15 and IL-7 were added to the complete medium to increase signal in the Elispot assay. Test antigens were diluted in complete medium, mixed with cells, and transferred to the Elispot plate. Subsequent to incubation in a CO₂ atmosphere typically at 37° C. the plates were assayed for cytokine spots using the above-mentioned Elispot kit following instructions from the manufacturer or equivalent procedures. Positive control qwew tetanus toxoid (TT) chloroform inactivated (for example TT from List Biological Laboratories Inc., catalog number 191 B) and CD3ε antibody (for example CD3ε from R&D Systems, catalog number MAB100). It is contemplated that several other positive controls can be used. Examples of positive control peptides are provided in Table 1.

TABLE 1

Examples of peptides used as positive control
for the Elispot assay. tt, tetanus toxin; dt,
diphtheria toxin; ebv, EBNA2 = Epstein-
Barr virus nuclear protein; NP, influenzae
nucleoprotein.

| | | |
|---|---|---|
| | | Description |
| Peptide name | Sequence | (aa #) |

| | | |
|---|---|---|
| TTD1 | IDKISDVSTIVPYIGPALNI | tt 633-652 |
| (SEQ ID NO:4) | | |
| DTD4 | AQSIALSSLMVAQAIPLVGEL | dt 330-350 |
| (SEQ ID NO:5) | | |
| EBV5 | TVFYNIPPMPL | ebv 280-290 |
| (SEQ ID NO:6) | | |
| TTD2 | FNNFTVSFWLRVPKVSASHLET | tt 947-968 |
| (SEQ ID NO:7) | | |
| TTZ | QYIKANSKFIGITELKKLE | tt 830-848 |
| (SEQ ID NO:8) | | |
| TTS | MQYIKANSKFIGITELK | tt 829-846 |
| (SEQ ID NO:9) | | |
| NP1 | DKGEIRRIWRGANNG | NP 112-126 |
| (SEQ ID NO:10) | | |
| NP2 | TRSAYERMCNILKGKFQTA | NP 215-233 |
| (SEQ ID NO:11) | | |
| NP3 | SLVGIDPFKLLQNSQVYSLIRP | NP 297-318 |
| (SEQ ID NO:12) | | |
| NP4 | LRVLSFIRGTKVSPRGKLSTRG | NP 341-362 |
| (SEQ ID NO:13) | | |

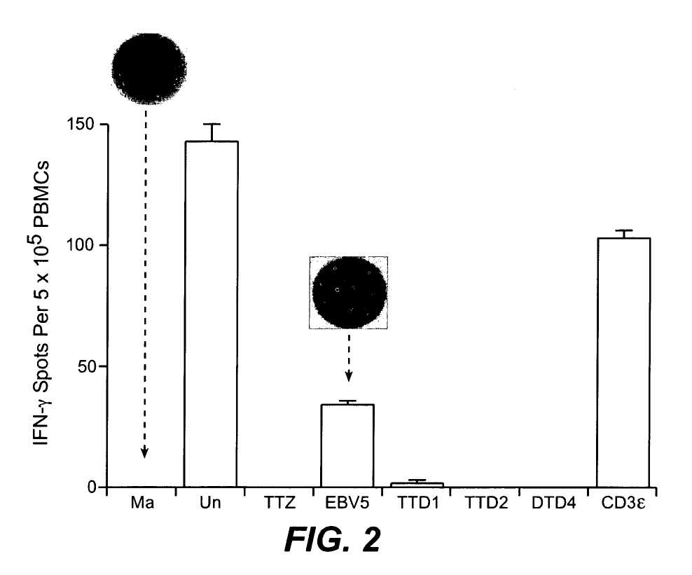

FIG. 2. Selection of donor/peptide combination for assay optimization. Elispot assay using PBMCs from donor 6622674. HLA II type: DRB1*0401*0701, DQA1*0201*030101, DQA1 *0103, DPB1*0201*1601, DRB4*01010101*0103, DQB1*0202*0302. Ma, negative control with no antigen added. Un, PBMCs from different donors mixed together. CD3e, antibody used as positive control. Experiments in duplicate.

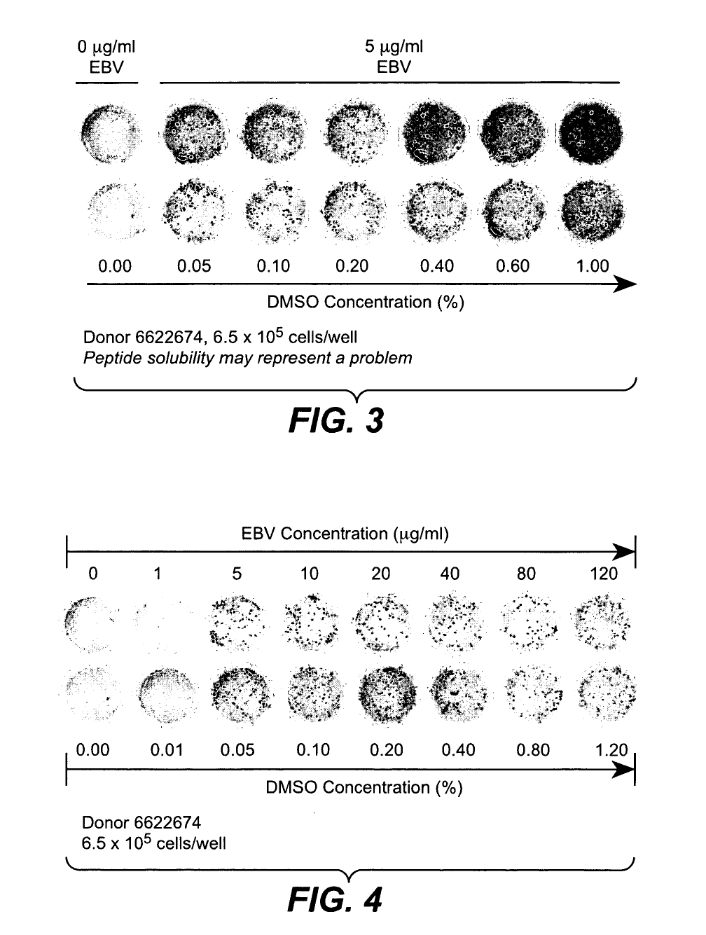

FIG. 3. Effect of various DMSO concentrations on the Elispot T cell activation assay using PBMCs from donor 6622674 at 6.5×10⁵ cells per well. EBV peptide concentration was either 0 μg/ml (negative control) or 5 μg/ml. Duplicate wells.

FIG. 4. Effect of various EBV peptide concentrations on the T cell activation assay using PBMCs from donor 6622674 at 6.5×10⁵ cells per well. The peptide was dissolved in DMSO, and increases of peptide concentration simultaneously increased the DMSO concentration in the medium. Duplicate wells.

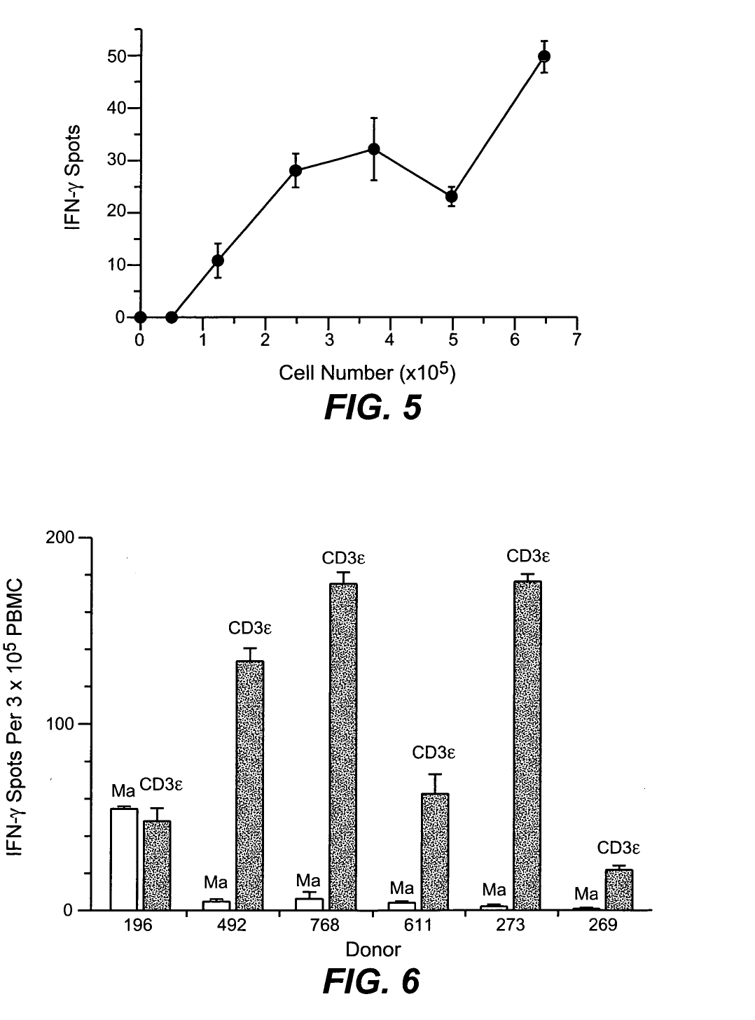

FIG. 5. Effect of cell number on the T cell activation Elispot assay using PBMCs from donor 6622674. 10 μg/ml EBV peptide. Triplicate or duplicate wells.

FIG. 6. T cell activation Elispot assay using several donors. The cell concentration is 3×10⁵ cells per well. CD3ε antibody, positive control; Ma, negative control with no antigen. Duplicate wells.

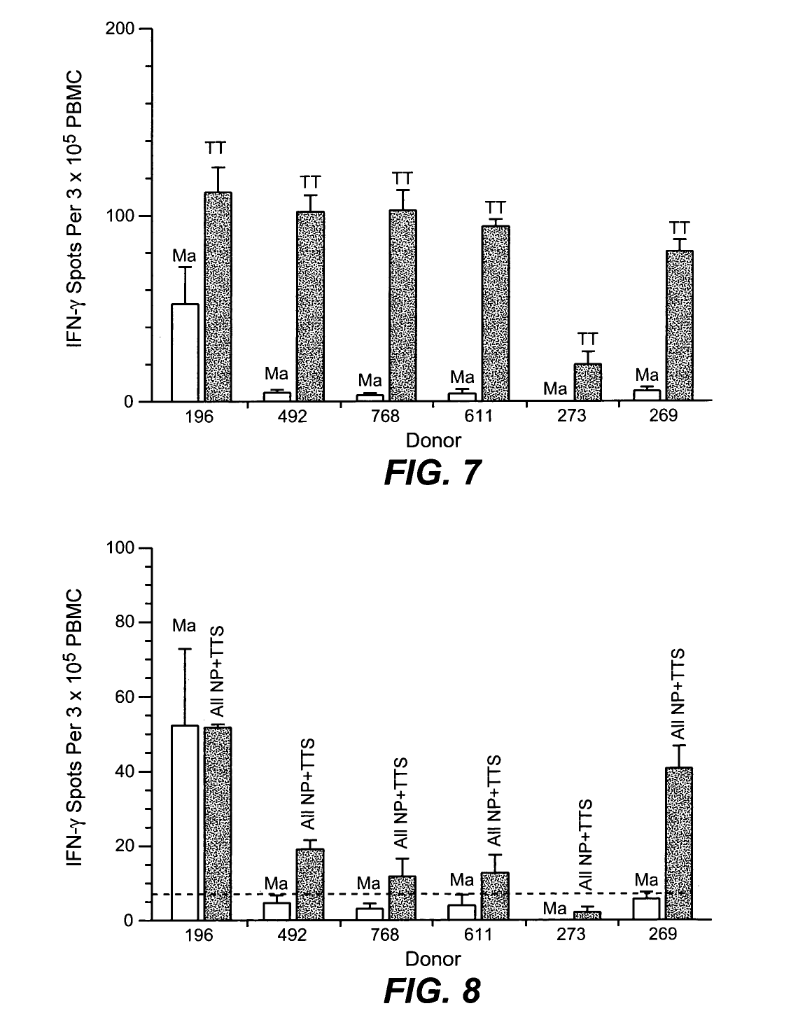

FIG. 7. T cell activation Elispot assay using several donors. The cell concentration is 3×10⁵ cells per well. TT, tetanus toxoid positive control; Ma, negative control with no antigen. Duplicate wells.

FIG. 8. T cell activation Elispot assay using several donors. The cell concentration is 3×10⁵ cells per well. All NP, NP1+NP2+NP3+NP4. Each peptide in the mix was at 10 μg/m. Ma, negative control with no antigen. Duplicate wells.

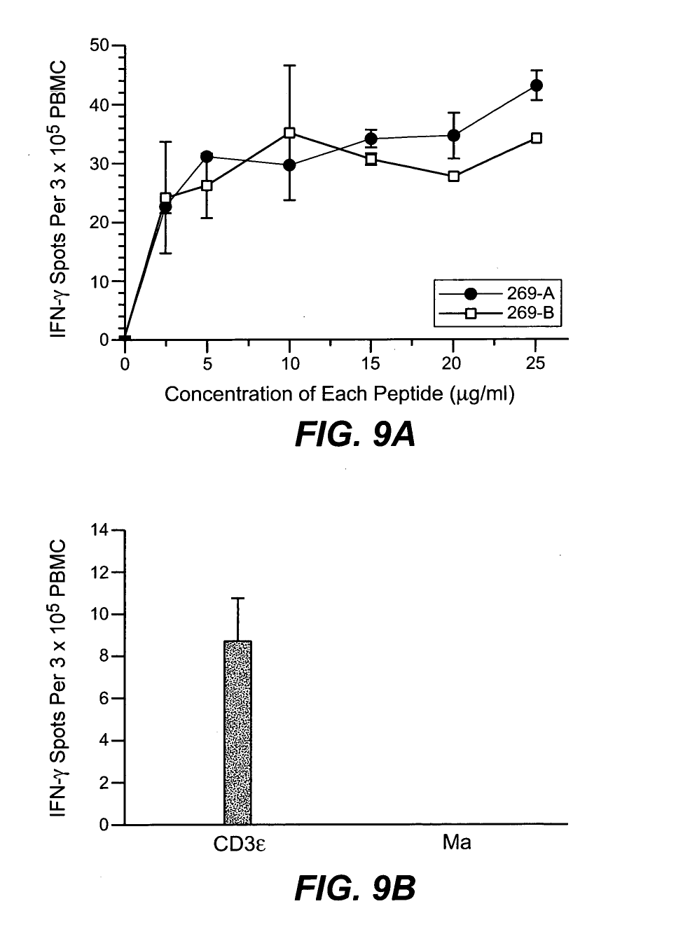

FIG. 9. Dose-response for a peptide mix (NP1+NP2+NP3+NP4) in a T cell activation Elispot assay. Donor FL68269. (A) 269-A and 269-B are different batches of the same donor. (B) Average values for the controls. Ma, negative control with no antigen; CD3c Ab, positive control. Duplicate wells.

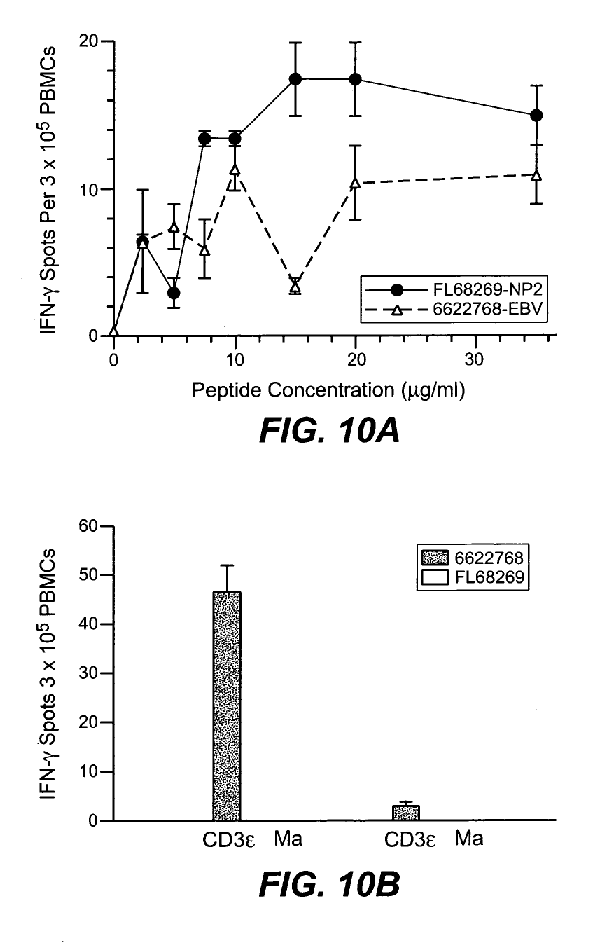

FIG. 10.(A) Dose-response for peptides in a T cell activation Elispot assay using two different peptide/donor combinations; (B) Average values for the controls. Ma, negative control with no antigen; CD3ε Ab, positive control. Duplicate wells.

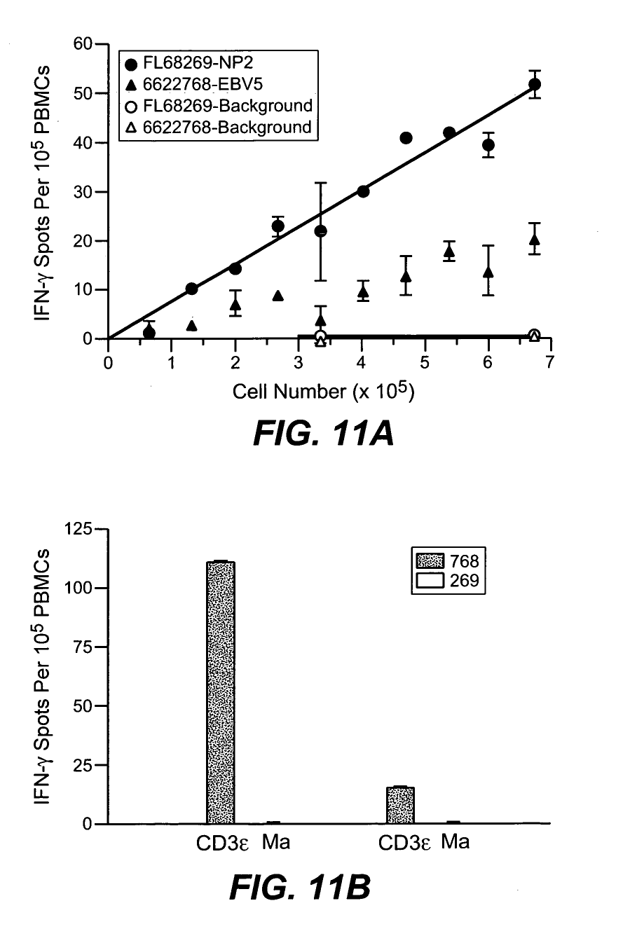

FIG. 11.(A) Effect of cell number on a T cell activation Elispot assay with two peptide/donor combinations. Peptide concentration of 10 μg/ml. The signal and the background are shown. (B) Average values for the controls. Ma, negative control with no antigen; CD3ε Ab, positive control.

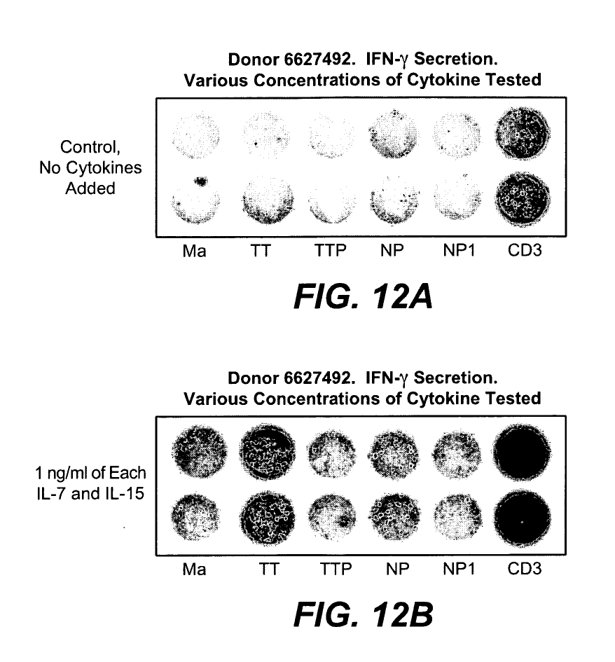

FIG. 12. Effect of added cytokines on a T cell activation Elispot assay. (A) control with no cytokines added; B, 1 ng/ml of each IL-7 and IL-15 added to the medium. Ma, negative control with no antigen. TT, tetanus toxoid and CD3ε antibody, positive controls. Donor 6627492: DRB1*1104 1301, DQA1*0103*0505, DPA1*0103*0201, DPB1*0101*0402, DRB3*0101*0202, DQB1*0301*0603. Duplicate wells.

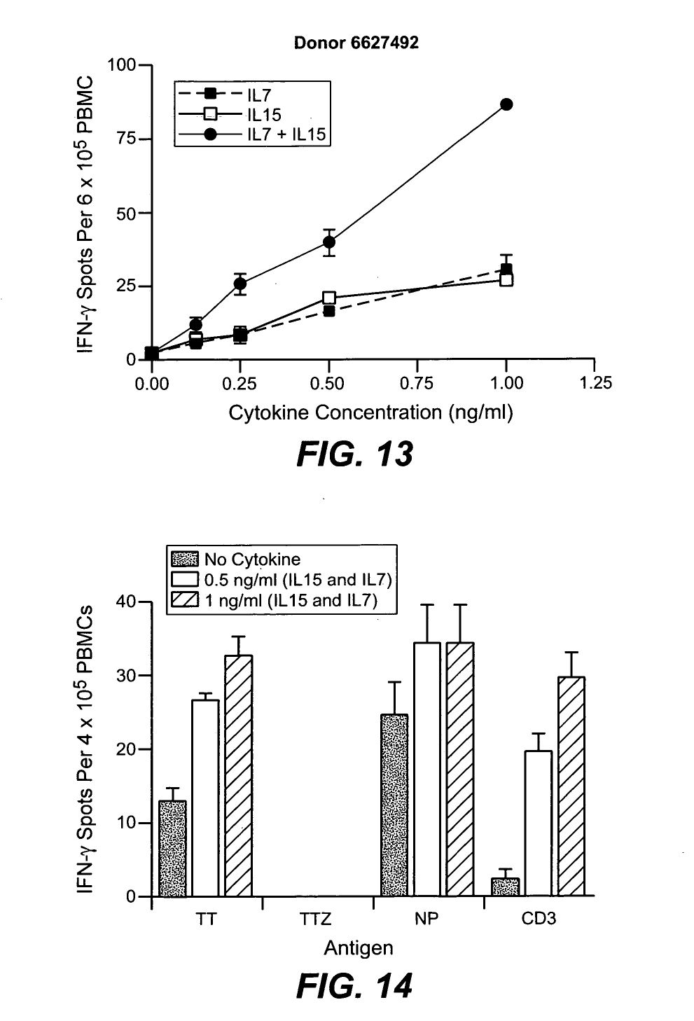

FIG. 13. Effect of added cytokines on a T cell activation Elispot assay. Dose response. Donor 6627492.

FIG. 14. Effect of added cytokines on a T cell activation Elispot assay. Donor FL68269. Ma, negative control with no antigen. TT (tetanus toxoid) and CD3ε antibody used as positive controls. Duplicate wells

IFN-β Antibody Binding Assay

Antibodies to IFN-β were detected to measure the immunogenic response of each subject to which IFN-β was administered. Serum obtained from blood was the source of antibody for the tests. For initial assay development goat anti-human IFN-β antibody was used (R&D Systems, catalog number AF814). In those instances protein G was used for detection.

Samples were tested for the presence of anti-IFN-β IgG using two different antibody binding assay formats, a direct capture ELISA (DCE) and an indirect capture ELISA (ICE). Betaseron® (Berlex) was reconstituted at 10 μM. For the DCE assay, MaxiSorp plates (Nalge Nunc International) were coated with 100 nM Betaseron® (20 μl per well) overnight at 4° C. in sealed plates, followed by a blocking step for 2 h. The blocking reagent was 5% non-fat dry milk in PBS with 0.05% Tween 20. Blocking and subsequent steps were performed at room temperature. The blocking reagent was replaced with plasma samples from human subjects and the plates incubated for 1 h. For the primary screen, a plasma dilution of 1:10 was used. The plates were then washed 3 times with PBS containing 0.05% Tween 20 (washing solution). 20 pi of secondary Eu-labeled anti-human IgG diluted 1:500 in DELPHIA assay buffer (Perkin-Elmer) was added to each well and the plate incubated for 1 h, followed by 3 washes with the washing solution above. 50 μl of DELFIA-enhancement solution (Perkin-Elmer) was added to each well and incubated for 10 min, then signal was detected in a Fusion-Alpha reader (Perkin-Elmer). To correct for inter-assay variations, samples yielding high signal and no signal were selected as positive (C+) and negative (C−) controls, respectively [A. R. Mire-Sluis et al. 2004. Recommendations for the design and optimization of immunoassays used in the detection of host antibodies against biotechnology products. J. Immunol. Methods 289: 1-16, entirely incorporated by reference]. The C+ and C− samples were included in all plates. The results were expressed as the ratio sample signal/C+ signal. For the ICE assay, MaxiSorp plates were first coated with the monoclonal anti-human IFN-β IgG antibody BO2 (Yamasa-Shoyu Co.) as previously described [A. R. Pachner. 2004. An improved ELISA for screening for neutralizing anti-IFN-beta antibodies in MS patients. Neurology, 61: 1444-1446, entirely incorporated by reference], and plasma samples from human subjects incubated in the plates for 2 h. Other steps were performed as described above for the DCE assay.

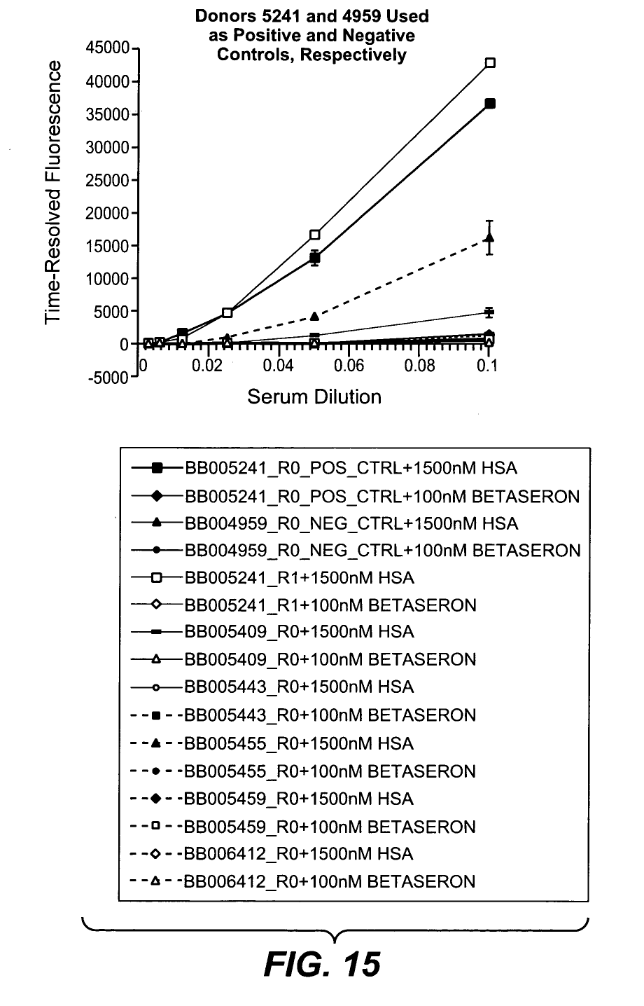

FIG. 15 shows te results from DELFIA assay (DCE) probing for IFNb-reactive IgGs in Betaseron treated patient serum samples. Donors are denoted with the last three digits of their identifier code.

TABLE 2
Association between MHC class II alleles and production of anti-IFN-β antibodies in
multiple sclerosis patients undergoing Betaseron therapy

| Patient | | | | Anti-IFN-β Ab | Anti-IFN-β Ab | Anti-IFN-β Ab | Anti-IFN-β Ab |
|---|---|---|---|---|---|---|---|
| number | DRB1 | DQA1 | DQB1 | ratio (DCE)^{a} | (DCE)^{a} | ratio (ICE)^{b} | (ICE)^{b} |
| 5449 | 0701 | 0201 | 0202 | 3.98 ± 0.62 | + | 0.79 ± 0.07 | + |
| 5455 | 0701 1501 | 0201 0102 | 0202 0602 | 1.05 ± 0.26 | + | 0.17 ± 0.02 | + |
| 5241 | 0701 0404 | 0201 0301 | 0202 0302 | 0.97 ± 0.01 | + | 0.04 ± 0.01 | − |
| 6421 | 0701 1501 | 0201 0102 | 0202 0602 | 0.55 ± 0.07 | + | 0.13 ± 0.02 | + |
| 6424 | 0701 0101 | 0201 0101 | 0303 0501 | 0.40 ± 0.10 | + | 0.35 ± 0.02 | + |
| 5814 | 0701 0401 | 0201 0301 | 0202 0302 | 0.34 ± 0.07 | + | 0.12 ± 0.09 | + |
| 5818 | 0701 0101 | 0201 0101 | 0202 0501 | 0.11 ± 0.04 | + | 0.28 ± 0.02 | + |
| 4957 | 0701 1501 | 0201 0102 | 0202 0602 | 0.07 ± 0.00 | + | 0.53 ± 0.02 | + |
| 5446 | 0701 0404 | 0201 0301 | 0202 0302 | 0.00 ± 0.00 | − | 0.07 ± 0.01 | − |
| 5168 | 1301 1501 | 0102 0103 | 0602 0603 | 6.97 ± 0.70 | + | 0.90 ± 0.06 | + |
| 6429 | 1301 1501 | 0102 0103 | 0602 0603 | 0.56 ± 0.13 | + | 0.13 ± 0.01 | + |
| 6427 | 1301 0301 | 0103 0501 | 0201 0603 | 0.41 ± 0.12 | + | 0.34 ± 0.03 | + |
| 6432 | 1301 0401 | 0103 0301 | 0302 0603 | 0.24 ± 0.02 | + | 1.00 ± 0.12 | + |
| 6428 | 1301 1501 | 0102 0103 | 0602 0603 | 0.00 ± 0.01 | − | 0.23 ± 0.02 | + |
| 5402 | 0101 0401 | 0101 0303 | 0301 0501 | 0.47 ± 0.10 | + | 0.96 ± 0.03 | + |
| 6422 | 1501 | 0102 | 0602 | 1.44 ± 0.19 | + | 0.75 ± 0.06 | + |
| 5458 | 1501 1101 | 0102 0505 | 0301 0602 | 0.68 ± 0.10 | + | 0.14 ± 0.02 | + |
| 5809 | 1501 | 0102 | 0602 | 0.29 ± 0.08 | + | 0.18 ± 0.01 | + |
| 5409 | 1501 0901 | 0102 0302 | 0303 0602 | 0.28 ± 0.04 | + | 0.19 ± 0.02 | + |
| 6425 | 1501 1101 | 0102 0505 | 0301 0602 | 0.03 ± 0.01 | − | 0.29 ± 0.05 | + |
| 5816 | 1501 0801 | 0102 0401 | 0402 0602 | 0.02 ± 0.00 | − | 0.09 ± 0.01 | + |
| 5815 | 1501 | 0102 | 0602 | 0.00 ± 0.00 | − | 0.19 ± 0.02 | + |
| 4959 | 1501 | 0102 | 0602 | 0 ± 0 | − | 0.05 ± 0.01 | − |
| 4960 | 1501 0801 | 0102 0401/04 | 0402 0602 | 0 ± 0 | − | 0.02 ± 0.01 | − |
| 5453 | 1501 0101 | 0101 0102 | 0501 0602 | 0 ± 0 | − | 0.05 ± 0.01 | − |
| 5454 | 1501 0404 | 0102 0301 | 0302 0602 | 0 ± 0 | − | 0.03 ± 0.01 | − |
| 6410 | 1501 0401 | 0102 0303 | 0301 0602 | 0 ± 0 | − | 0.09 ± 0.01 | + |
| 6412 | 1501 0405 | 0102 0303 | 0202 0602 | 0 ± 0 | − | 0.04 ± 0.01 | − |
| 6423 | 1501 1601 | 0102 0102 | 0502 0602 | 0 ± 0 | − | 0.03 ± 0.01 | − |
| 5808 | 1501 1101 | 0102 0505 | 0301 0602 | 0 ± 0 | − | 0.04 ± 0.01 | − |
| 5811 | 1501 0801 | 0102 0401 | 0402 0602 | 0 ± 0 | − | 0.03 ± 0.01 | − |
| 6408 | 0301 1104 | 0505 05AE^{c} | 0201 0301 | 0.03 ± 0.01 | − | 0.04 ± 0.01 | − |
| 6414 | 0301 1101 | 0505 05AE^{c} | 0201 0301 | 0.02 ± 0.01 | − | 0.08 ± 0.01 | − |
| 5819 | 0301 1501 | 0102 0501 | 0201 0602 | 0.01 ± 0.01 | − | 0.05 ± 0.01 | − |
| 5456 | 0301 1501 | 0102 0501 | 0201 0602 | 0.01 ± 0.01 | − | 0.05 ± 0.01 | − |
| 5443 | 0301 1102 | 0501 0505 | 0201 0301 | 0 ± 0 | − | 0.03 ± 0.01 | − |
| 6417 | 0301 12AG^{c} | 0505 05AE^{c} | 0201 0301 | 0 ± 0 | − | 0.02 ± 0.01 | − |
| 5451 | 0404 1302 | 0102 0301 | 0302 0604 | 0.01 ± 0.00 | − | 0.18 ± 0.02 | + |
| 5817 | 0101 0801 | 0101 0401 | 0402 0501 | 0 ± 0 | − | 0.03 ± 0.03 | − |

^{a}Ratio values ≧ 0.07 indicate a positive (+) result for anti-IFN-β binding antibodies in the direct capture ELISA (DCE).
^{b}Ratio values ≧ 0.08 indicate a positive (+) result for anti-IFN-β binding antibodies in the indirect capture ELISA (ICE).
^{c}DRB1*12AG includes DRB1*1201, 1206. DQA1*05AE includes DQA1*0501, 0505.

Three or more independent determinations of anti IFN β antibody ratios were done for each of the 39 patient samples with both DCE and ICE; values are mean ± standard deviation.

Table 2 shows the correlation between different MHC alleles and an immunogenic response. As can be observed, certain alleles corresponded to a higher immunogenic response than other alleles.

### Example 3

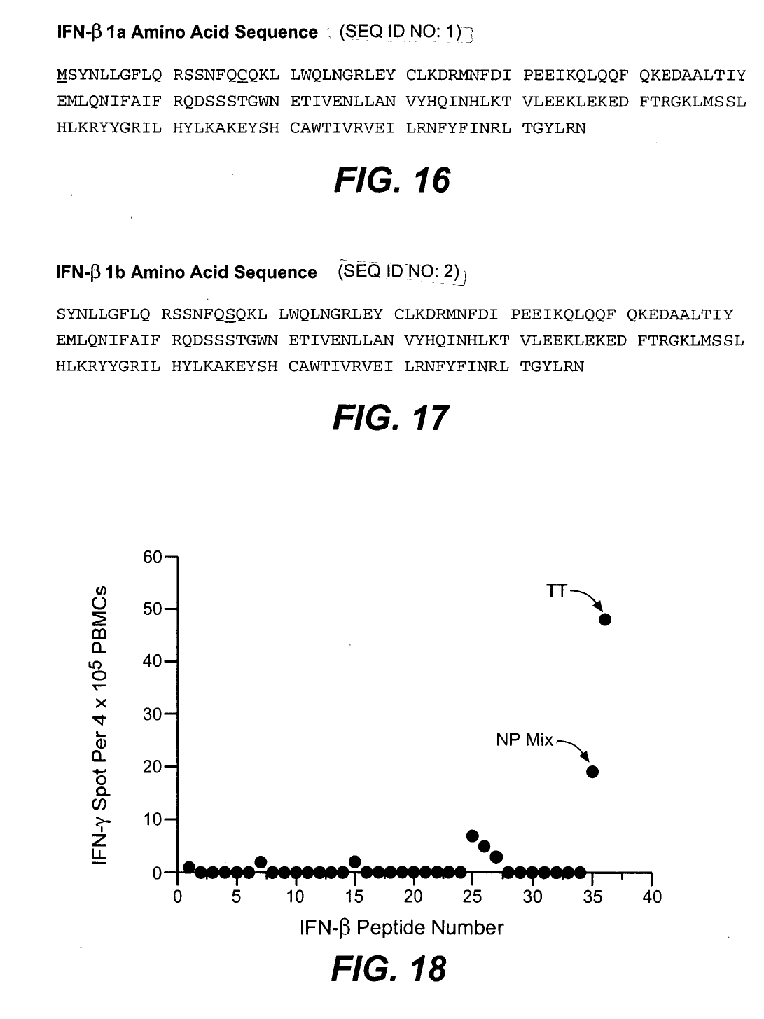

Overlapping peptides covering the entire protein sequence are used for epitope mapping. FIG. 16 shows IFN-β 1a amino acid sequence. FIG. 17 shows that IFN-β 1b amino acid sequence.

TABLE 3
IFN-β peptides.

| Peptide | |
|---|---|
| names | Peptide sequences |
| IB1 | MSYNLLGFLQRSSNFQC (SEQ ID NO:14) |
| IB2 | LGFLQRSSNFQCQKLLW (SEQ ID NO:15) |
| IB3 | RSSNFQCQKLLWQLNGR (SEQ ID NO:16) |
| IB4 | FQCQKLLWQLNGRLEYC (SEQ ID NO:17) |
| IB5 | LWQLNGRLEYCLKDRMN (SEQ ID NO:18) |
| IB6 | GRLEYCLKDRMNFDIPE (SEQ ID NO:19) |
| IB7 | CLKDRMNFDIPEEIKQL (SEQ ID NO:20) |
| IB8 | MNFDIPEEIKQLQQFQK (SEQ ID NO:21) |
| IB9 | PEEIKQLQQKQKEDAAL (SEQ ID NO:22) |
| IB10 | KQLQQFQKEDAALTIYE (SEQ ID NO:23) |
| IB11 | FQKEDAALTIYEMLQNI (SEQ ID NO:24) |
| IB12 | ALTIYEMLQNIFAIFRQD (SEQ ID NO:25) |
| IB13 | EMLQNIFAIFRQDSSST (SEQ ID NO:26) |
| IB14 | IFAIFRQDSSSTGWNET (SEQ ID NO:27) |
| IB15 | RQDSSSTGWNETIVENL (SEQ ID NO:28) |
| IB16 | STGWNETIVENLLANVY (SEQ ID NO:29) |
| IB17 | ETIVENLLANVYHQINH (SEQ ID NO:30) |
| IB18 | NLLANVYHQINHLKTVL (SEQ ID NO:31) |
| IB19 | VYHQINHLKTVLEEKLE (SEQ ID NO:32) |
| IB20 | NHLKTVLEEKLEKEDFT (SEQ ID NO:33) |
| IB21 | VLEEKLEKEDFTRGKLM (SEQ ID NO:34) |
| IB22 | LEKEDFTRGKLMSSLHL (SEQ ID NO:35) |
| IB23 | FTRGKLMSSLHLKRYYG (SEQ ID NO:36) |
| IB24 | LMSSLHLKRYYGRILHY (SEQ ID NO:37) |
| IB25 | HLKRYYGRILHYLKAKE (SEQ ID NO:38) |
| IB26 | YGRILHYLKAKEYSHCA (SEQ ID NO:39) |
| IB27 | HYLKAKEYSHCAWTIVR (SEQ ID NO:40) |
| IB28 | KEYSHCAWTIVRVEILR (SEQ ID NO:41) |
| IB29 | HCAWTIVRVEILRNFYF (SEQ ID NO:42) |
| IB30 | VRVEILRNFYFINRLTG (SEQ ID NO:43) |
| IB31 | LRNFYFINRLTGYLRN (SEQ ID NO:44) |
| IB1b32 | SYNLLGFLQRSSNFQCQ (SEQ ID NO:45) |
| IB1b33 | LGFLQRSSNFQSQKLLW (SEQ ID NO:46) |
| IB-34 | SSLHLKRYYGRILHY (SEQ ID NO:47) |

Epitope mapping using the PBMC T cell activation assay described herein was used to identify epitopes recognized by the antibodies in the IFN-β protein. Normal donors not on IFN-β therapy can be used as negative controls for the assay, to investigate if there is unspecific T cell activation.

TABLE 4

Composition of IFN-β peptide pools. Each
pool contained three overlapping peptides
except for pool 10 (4 peptides). The final
concentration of each peptide in the
Elispot assay was 10 μg/ml.

| SEQ | | |
|---|---|---|
| ID | | |
| NO: | | |
| 14 | IB1-MSYNLLGFLQRSSNFQC | Pool 1 |
| 15 | IB2-LGFLQRSSNFQCQKLLW | |
| 16 | IB3-RSSNFQCQKLLWQLNGR | |
| 17 | IB4-FQCQKLLWQLNGRLEYC | Pool 2 |
| 18 | IB5-LWQLNGRLEYCLKDRMN | |
| 19 | IB6-GRLEYCLKDRMNFDIPE | |
| 20 | IB7-CLKDRMNFDIPEEIKQL | Pool 3 |
| 21 | IB8-MNFDIPEEIKQLQQFQK | |
| 22 | IB9-PEEIKQLQQFQKEDAAL | |
| 23 | IB10-KQLQQFQKEDAALTIYE | Pool 4 |
| 24 | IB11-FQKEDAALTIYEMLQNI | |
| 25 | IB12-ALTIYEMLQNIFAIFRQD | |
| 26 | IB13-EMLQNIFAIFRQDSSST | Pool 5 |
| 27 | IB14-IFAIFRQDSSSTGWNET | |
| 28 | IB15-RQDSSSTGWNETIVENL | |
| 29 | IB16-STGWNETIVENLLANVY | Pool 6 |
| 30 | IB17-ETIVENLLANVYHQINH | |
| 31 | IB18-NLLANVYHQINHLKTVL | |
| 32 | IB19-VYHQINHLKTVLEEKLE | Pool 7 |
| 33 | IB20-NHLKTVLEEKLEE)FT | |
| 34 | IB21-VLEEKLEKEDFTRGKLM | |
| 35 | IB22-LEKEDFTRGKLMSSLHL | Pool 8 |
| 36 | IB23-FTRGKLMSSLHLKRYYG | |
| 37 | IB24-LM SSLHLKRYYGRILHY | |
| 38 | IB25-HLKRYYGRILHYLKAKE | Pool 9 |
| 39 | IB26-YGRILHYLKAKEYSHCA | |
| 40 | IB27-HYLKAKEYSHCAWTIVR | |
| 41 | IB28-KEYSHCAWTIVRVEILR | Pool 10 |
| 42 | IB29-HC AWTIVRVEILRNFYF | |
| 43 | IB30-VRV EI LRNFYFINRLTG | |
| 44 | IB31-LRNFYFINRLTGYLRN | |

FIG. 18. Peptide scan with samples from a normal donor, not in IFN-β therapy. IL-15 and IL-7 added to the medium at 1 ng/ml. The background from the negative control without antigen was subtracted. Donor 6627492.

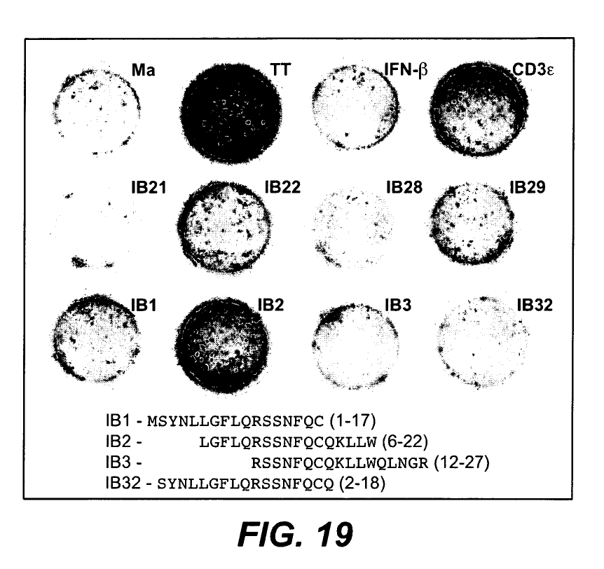

FIG. 19. IFN-β epitope mapping using a T cell activation Elispot assay. IL-15 and IL-7 added to the medium at 1 ng/ml. Donor B005241, DRB1*0404*0701. Ma, negative control with no antigen; IFN-b, Betaseron; TT (tetanus toxoid) and CD3ε antibody uses as positive control. Donor was positive for IFN-β binding antibody assay.

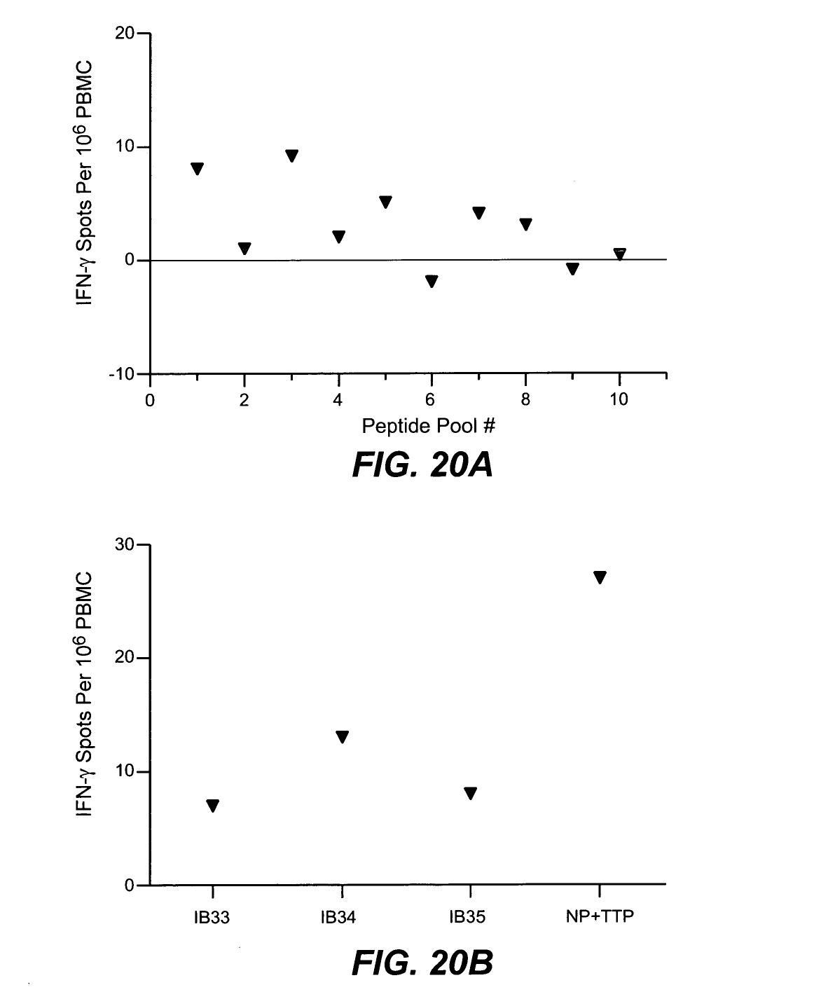

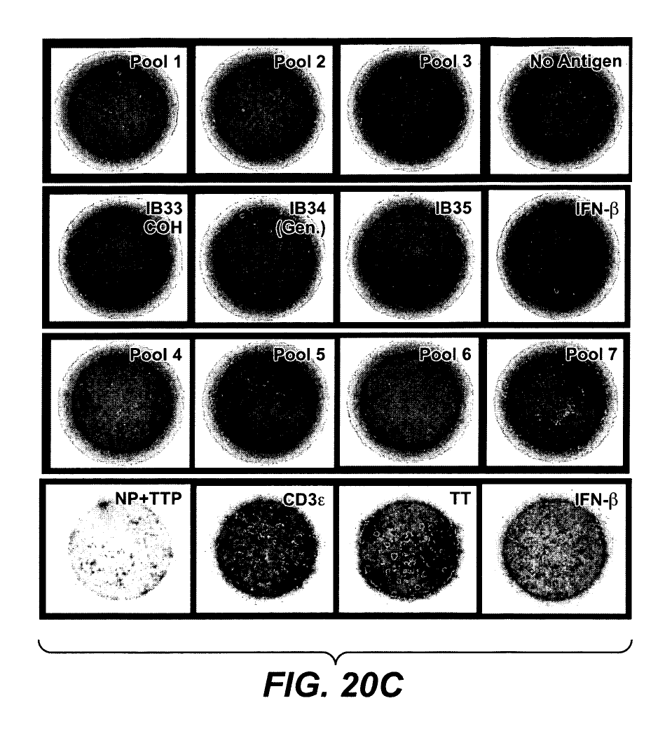

FIG. 20. IFN-β epitope mapping using a T cell activation Elispot assay and PBMC from donor B006412. Donor was negative for IFN-β binding antibody assay. IL-15 and IL-7 added to the medium at 2.5 ng/ml. IFN-b, Betaseron; TT (tetanus toxoid) and CD3ε antibody used as positive controls. FIG. 20(A) pools containing 3 peptides tested at 10 μg/ml each FIG. 20(B) individual peptides at 10 μg/ml concentration and a pool of control peptides (NP+TTP) tested for T cell activation FIG. 20(C) pictures of wells assayed in the presence of IFN-β peptide pools or controls. In FIG. 20(A) and (B) the background from the negative control without antigen was subtracted.

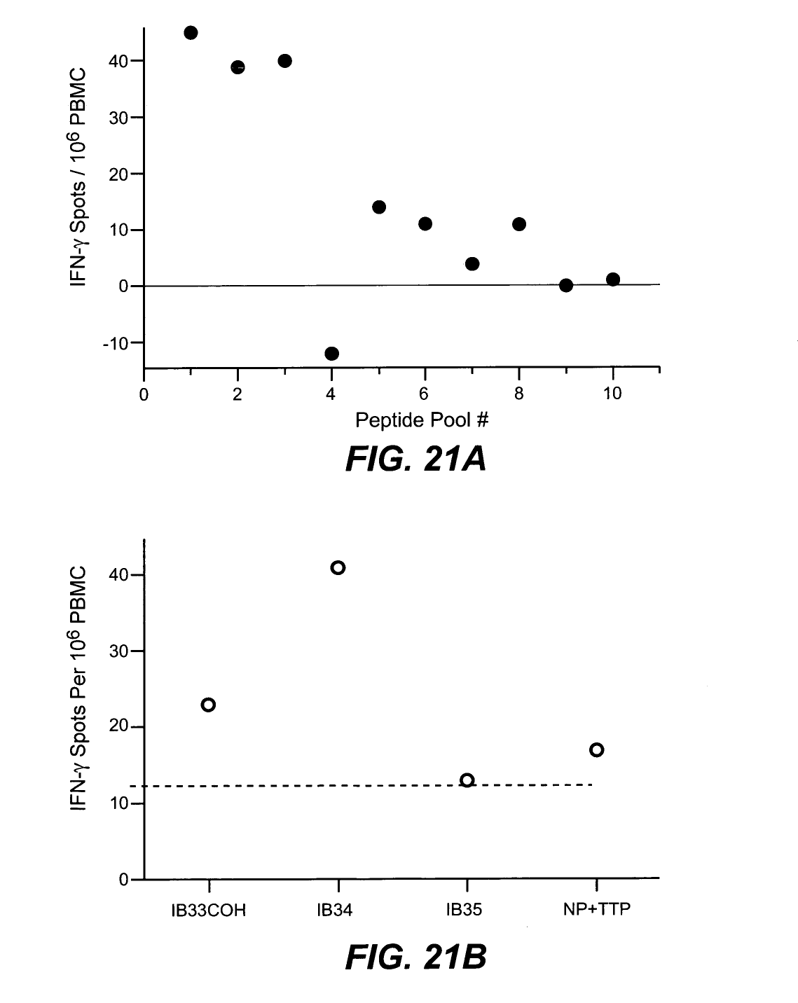

FIG. 21. IFN-β epitope mapping using a T cell activation Elispot assay and PBMC from donor B005409. Donor was positive for IFN-β binding antibody assay. IL-15 and IL-7 added to the medium at 2.5 ng/ml. IFN-b, Betaseron; TT (tetanus toxoid) and CD3ε antibody uses as positive control. FIG. 21(A) pools containing 3 peptides tested at 10 μg/ml each, FIG. 21(B) individual peptides at 10 μg/ml concentration and a pool of control peptides (NP+TTP) tested for T cell activation, FIG. 21(C) pictures of wells assayed in the presence of IFN-β peptide pools or controls. In FIG. 21(A) and (B) the background from the negative control without antigen was subtracted.

### Example 5

HLA-typing of MS patients testing positive or negative for IFN-β antibodies allowed investigation of the association between MHC class II binding epitopes and IFN-β immunogenicity. Results with positive controls in the PBMC Elispot assay indicated that the donors were presenting a normal immune-response.

TABLE 5
Summary of results for multiple sclerosis patients on IFN-β therapy: HLA
typing and the development of IFN-β antibodies. The IFN-β neutralizing
antibody assay (NabFeron ®) was performed by Athena Diagnostics.

| | | | NabFeron ® | MS | | |
|---|---|---|---|---|---|---|
| | HLA | IFN-β Ab- | (IFN-β) Ab | diagnosis | Betaseron | |
| | type: | binding | assay (Nab | date and | therapy | |
| Donor | DRB1 | assay | titer) | type | start date | Dosage |
| BB005455 | *0701 | (+) | <20 (normal) | 2001 | July 2003 | 0.25 mg |
| | *1501 | | | RR | | q.o.d. |
| BB005409 | *1501 | (+) | <20 (normal) | 1974 | 1999 | 0.15 mg |
| | *090102 | | | SPWR | | q.o.d. |
| BB005241 | *0404 | (++) | <20 (normal) | 1989, | 1994 | 0.15 mg |
| | *0701 | | | RR | | q.o.d. |
| BB005168 | *1301 | (+++++++) | 195 (highly | Jul. 01, 2002 | Jul. 01, 2002 | 0.15 mg |
| | *1501 | | elevated) | | | q.o.d. |
| BB004959 | *1501 | (−) | <20 (normal) | 1987, | 1998 | 0.15 mg |
| | | | | RR | | q.o.d. |

Q.o.d., every other day;
RR, relapsing remitting;
SPWR, secondary progressive without continued relapse.

One subject, BB005168, showed a highly elevated IFN-β antibody response. Others showed a slightly elevated IFN-β antibody response.

### Example 6

TABLE 6
High resolution HLA typing data for multiple sclerosis patients undergoing IFN-beta therapy
and the corresponding anti IFN-beta binding antibody results for the human subjects.

| Human | IFN-β Ab | | | | | | |
|---|---|---|---|---|---|---|---|
| subject | ratio | DRB1 | DQA1 | DQB1 | DPA1 | DPB1 | DRB(3, 4, 5) |
| BB004959 | 0.00 ± 0.00 | *1501 | *0102 | *0602 | *0103 | *0401 | DRB5 *010101 |
| BB005168 | 7.32 ± 0.48 | *1301 | *0102 | *0602 | *0103 | *0401 | DRB3 |
| | | *1501 | *0103 | *0603 | | | *0202 DRB5 |
| | | | | | | | *010101 |
| BB005241 | 0.58 ± 0.06 | *0404 | *0201 | *0202 | *0103 | *0401 | DRB4 |
| | | *0701 | *0301 | *0302 | | *0402 | *0103 |
| BB005409 | 0.30 ± 0.05 | *1501 | *0102 | *0303 | *0103 | *0401 | DRB4 |
| | | *090102 | *0302 | *0602 | | *0402 | *01030102N |
| | | | | | | | DRB5 *010101 |
| BB005455 | 1.11 ± 0.26 | *0701 | *0102 | *0202 | *0103 | *0401 | DRB4 |
| | | *1501 | *0201 | *0602 | | *4801 | *0103 DRB5 |
| | | | | | | | *010101 |
| BB004957 | 0.08 ± 0.00 | *0701 | *0102 | *0202 | *0103/07 | *0301 | DRB4 |
| | | *1501 | *0201 | *0602 | | *0401 | *0101 DRB5 |
| | | | | | | | *010101 |
| BB005443 | 0.00 ± 0.00 | *0301 | *0501 | *0201 | *0103 | *0101 | DRB3 |
| | | *1102 | *0505 | *0301 | *0201 | *1501 | *0101 DRB3 |
| | | | | | | | *0202 |
| BB004960 | 0.00 ± 0.00 | *0801 | *0102 | *0402 | *0103 | *0401 | DRB5 |
| | | *1501 | *0401/04 | *0602 | | | *010101 |
| BB006412 | 0.00 ± 0.00 | *0405 | *0102 | *0202 | *0103/07 | *0402 | DRB4 |
| | | *1501 | *0303 | *0602 | *0201 | *1301 | *0103 DRB5 |
| | | | | | | | *010101 |
| BB006410 | 0.00 ± 0.00 | *0401 | *0102 | *0301 | *0103 | *0401 | DRB4 |
| | | *1501 | *0303 | *0602 | | | *0103 DRB5 |
| | | | | | | | *010101 |
| BB006425 | 0.04 ± 0.01 | *1101 | *0102 | *0301 | *0103 | *0201 | DRB3 |
| | | *1501 | *0505 | *0602 | *0201 | *0402 | *0202 DRB5 |
| | | | | | | | *010101 |
| BB006432 | 0.26 ± 0.03 | *0401 | *0103 | *0302 | *0103 | *0401 | DRB3 |
| | | *1301 | *0301 | *0603 | | *0402 | *0202 DRB4 |
| | | | | | | | *0103 |
| BB005402 | 0.50 ± 0.09 | *0101 | *0101 | *0301 | *0103 | *0201 | DRB4 |
| | | *0401 | *0303 | *0501 | | *1601 | *0103 |
| BB005454 | 0.00 ± 0.00 | *0404 | *0102 | *0302 | *0103 | *0401 | DRB4 |
| | | *1501 | *0301 | *0602 | | | *01030102N |
| | | | | | | | DRB5 |
| | | | | | | | *010101 |
| BB005456 | 0.01 ± 0.02 | *0301 | *0102 | *0201 | *0103 | *0401 | DRB3 |
| | | *1501 | *0501 | *0602 | | | *0102 DRB5 |
| | | | | | | | *010101 |
| BB005453 | 0.00 ± 0.00 | *0101 | *0101 | *0501 | *0201 | *0501 | DRB5 |
| | | *1501 | *0102 | *0602 | *0202 | *1001 | *010101 |
| BB006429 | 0.59 ± 0.14 | *1301 | *0102 | *0602 | *0103 | *0402 | DRB3 |
| | | *1501 | *0103 | *0603 | | | *0202 DRB5 |
| | | | | | | | *010101 |
| BB006423 | 0.00 ± 0.00 | *1501 | *0102 | *0502 | *0103 | *0401 | DRB5 |
| | | *1601 | | *0602 | | *0402 | *010101 DRB5 |
| | | | | | | | *0202 |
| BB006422 | 1.51 ± 0.17 | *1501 | *0102 | *0602 | *0103 | *0201 | DRB5 |
| | | | | | | *0401 | *010101 |
| BB006424 | 0.42 ± 0.10 | *0101 | *0101 | *0303 | *0103 | *0301 | DRB4 |
| | | *0701 | *0201 | *0501 | | *0401 | *01030102N |
| BB006428 | 0.01 ± 0.01 | *1301 | *0102 | *0602 | | | |
| | | *1501 | *0103 | *0603 | | | |
| BB006421 | 0.58 ± 0.06 | *0701 | *0102 | *0202 | | | |
| | | *1501 | *0201 | *0602 | | | |
| BB005458 | 0.72 ± 0.11 | *1101 | *0102 | *0301 | | | |
| | | *1501 | *0505 | *0602 | | | |
| BB006408 | 0.03 ± 0.02 | *0301 | *0505 | *0201 | | | |
| | | *1104 | *05AE | *0301 | | | |
| BB006414 | 0.03 ± 0.02 | *0301 | *0505 | *0201 | | | |
| | | *1101 | *05AE | *0301 | | | |
| BB005819 | 0.02 ± 0.01 | *0301 | *0102 | *0201 | | | |
| | | *1501 | *0501 | *0602 | | | |
| BB006427 | 0.44 ± 0.12 | *0301 | *0103 | *0201 | | | |
| | | *1301 | *0501 | *0603 | | | |
| BB006417 | 0.00 ± 0.00 | *0301 | *0505 | *0201 | | | |
| | | *12AG | *05AE | *0301 | | | |
| BB005451 | 0.01 ± 001 | *0404 | *0102 | *0302 | | | |
| | | *1302 | *0301 | *0604 | | | |
| BB005817 | 0.00 ± 0.00 | *0101 | *0101 | *0402 | | | |
| | | *0801 | *0401 | *0501 | | | |
| BB005816 | 0.03 ± 0.01 | *0801 | *0102 | *0402 | | | |
| | | *1501 | *0401 | *0602 | | | |
| BB005446 | 0.06 ± 0.00 | *0404 | *0201 | *0202 | | | |
| | | *0701 | *0301 | *0302 | | | |
| BB005815 | 0.00 ± 0.01 | *1501 | *0102 | *0602 | | | |
| | | *1501 | *0102 | *06ANK | | | |
| BB005814 | 0.36 ± 0.07 | *0401 | *0201 | *0202 | | | |
| | | *0701 | *0301 | *0302 | | | |
| BB005809 | 0.30 ± 0.08 | *1501 | *0102 | *0602 | | | |
| | | *1501 | *0102 | *06ANK | | | |
| BB005808 | 0.00 ± 0.00 | *1101 | *0102 | *0301 | | | |
| | | *1501 | *0505 | *0602 | | | |
| BB005818 | 0.13 ± 0.05 | *0101 | *0101 | *0202 | | | |
| | | *0701 | *0201 | *0501 | | | |
| BB005811 | 0.00 ± 0.00 | *0801 | *0102 | *0402 | | | |
| | | *1501 | *0401 | *0602 | | | |
| BB005449 | 4.18 ± 0.58 | *0701 | *0201 | *0202 | | | |
| | | *0701 | *0201 | *0202 | | | |

### Example 7

The predicted immunogenic correlation between MHC alleles and IFN-β was measured by in vitro vaccination (IVV). The IVV assay was performed by introducing IFN-β to the APCs. A T cell response APC was determined by measuring IFN-γ production.

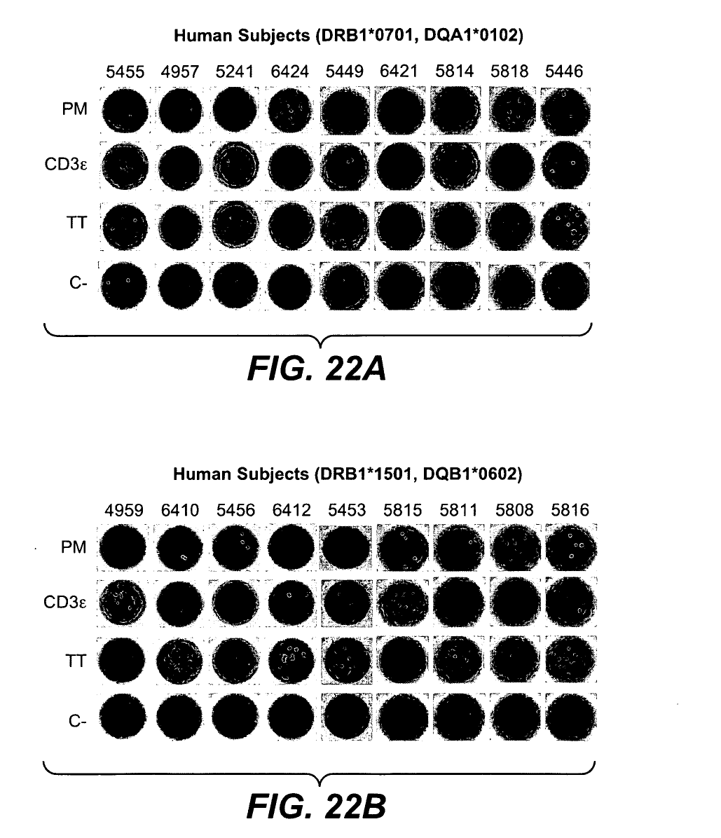

FIG. 22 shows IFN-γ production by T-cells tested with an Elispot assay. A, Human subjects containing the DRB1*0701 and DQA1*0201 alleles. B, Human subjects containing the DRB1*1501 and DQB1*0602 alleles and negative for antibody formation. IL-7 and IL-15 were added at a concentration of 2.5 ng/ml each. PM, mix of tetanus and influenzae nucleoprotein peptides; CD3ε, monoclonal antibody; TT, tetanus toxoid; C−, no antigen added.

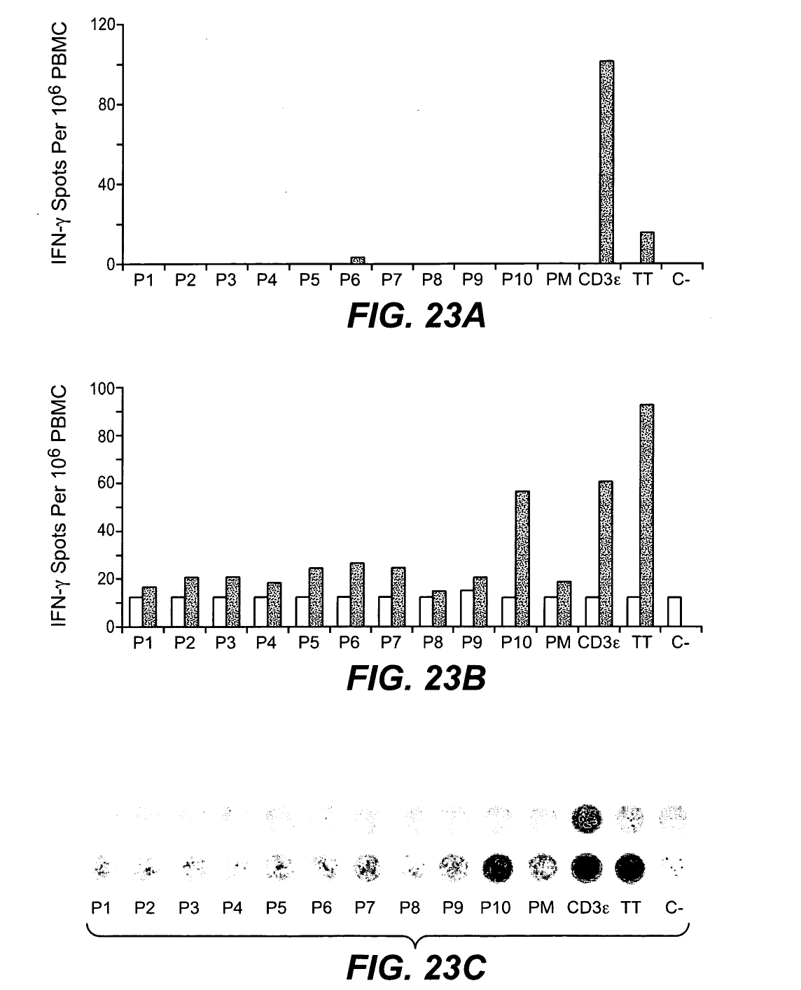

Potential epitopes of IFN-β were then mapped. FIG. 23 shows IFN-β epitope mapping using cells from human subject 4957 in an Elispot assay. IFN-γ production was measured in the presence of 0.5 ng/ml (A) or 2.5 ng/ml (B) of each IL-7 and IL-15. C, Corresponding pictures of the wells in the Elispot plate at 0.5 ng/ml (upper panel) or 2.5 ng/ml (lower panel) cytokine concentration. PM, mix of tetanus and influenzae nucleoprotein peptides; CD3E, monoclonal antibody; TT, tetanus toxoid; C−, no antigen added. P1 to P10, pools of IFN-β overlapping peptides. P1 to P9 contained 3 overlapping peptides each, starting from the N-terminus of the protein. P10 contained four overlapping peptides at the C-terminus.

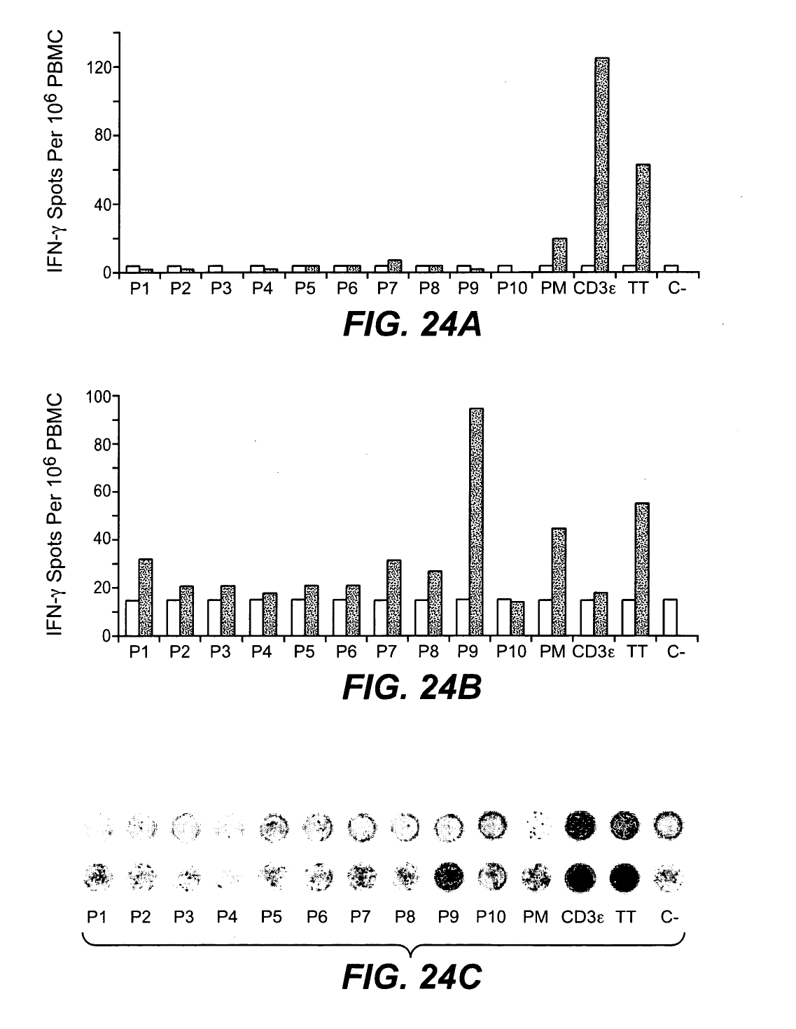

FIG. 24 shows IFN-β epitope mapping using cells from human subject 6424 in an Elispot assay. IFN-γ production was measured in the presence of 0.5 ng/ml (A) or 2.5 ng/ml (B) of each IL-7 and IL-15. C, Corresponding pictures of the wells in the Elispot plate at 0.5 ng/ml (upper panel) or 2.5 ng/ml (lower panel) cytokine concentration. PM, mix of tetanus and influenzae nucleoprotein peptides; CD3ε, monoclonal antibody; TT, tetanus toxoid; C−, no antigen added. P1 to P10, pools of IFN-β overlapping peptides. P1 to P9 contained 3 overlapping peptides each, starting from the N-terminus of the protein. P10 contained four overlapping peptides at the C-terminus.

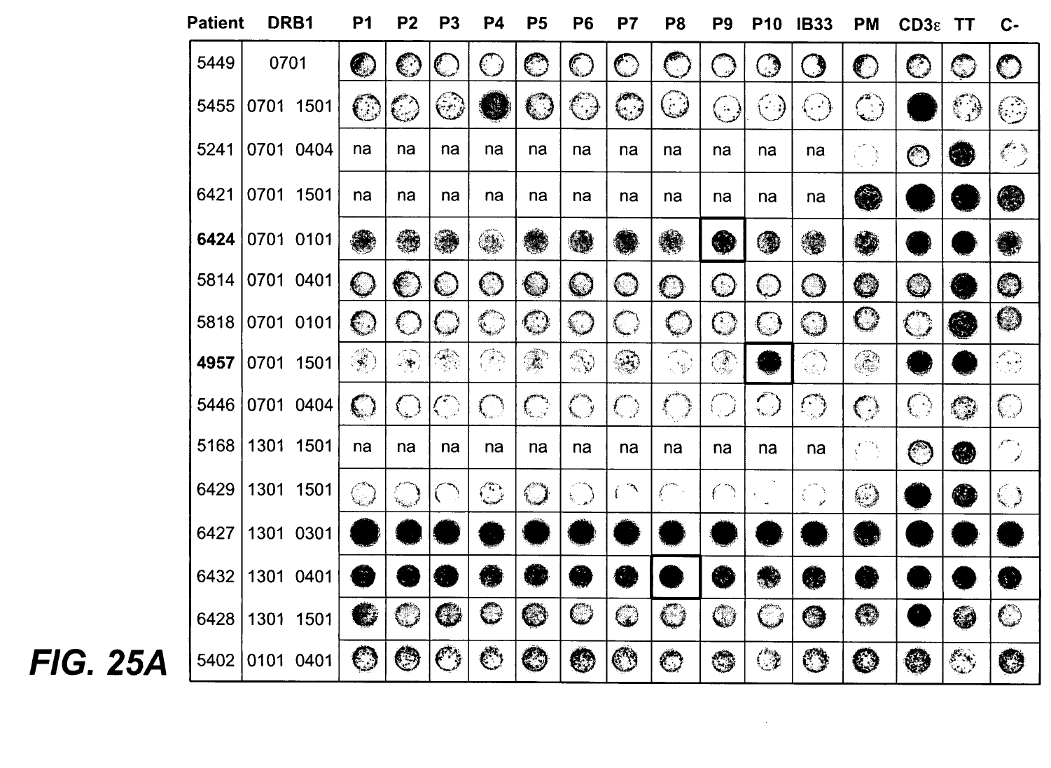

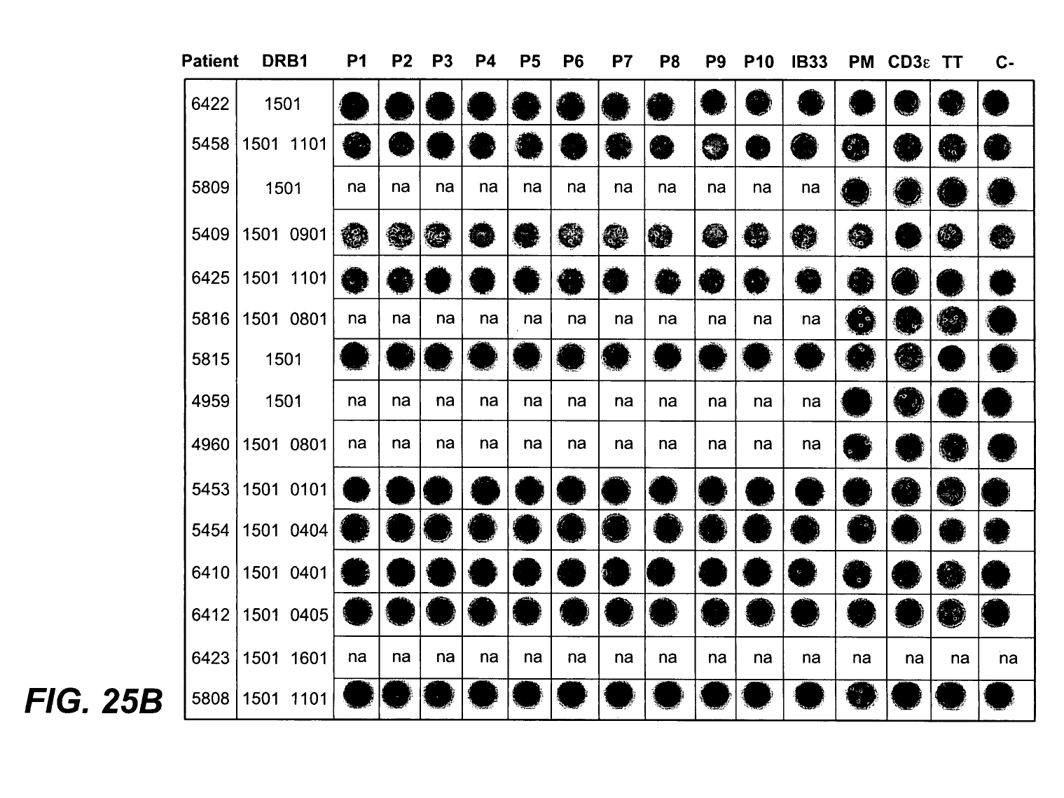

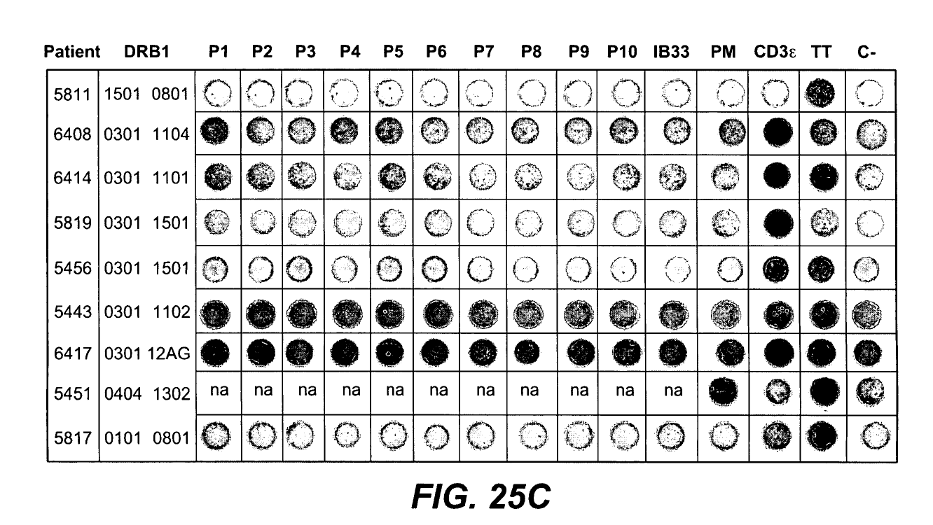

FIG. 25(A, B and C) shows results for an Elispot assay using PBMCs from both anti-interferon-beta antibody positive or negative subjects. IFN-γ production was measured in the presence of 2.5ng/ml each of IL-7 and IL-15. PM, mix of tetanus and influenzae nucleoprotein peptides; CD3ε, monoclonal antibody; TT, tetanus toxoid; C−, negative control (no antigen added).

### Example 8

### Peptide Binding Assay

Peptide binding to MHC class II proteins on the surface of Epstein-Barr virus-transformed B lymphocytes was tested by quantitative flow cytometric analysis (Busch et al. 1990), with modifications. Low resolution HLA typed B cell lines (DR2 and DR7) were obtained from the Coriell Institute for Medical Research (Camden, N.J.). Those cell lines were HLA typed at high resolution for DRB1, DQA1 and DQB1. The B cell lines GM03107, GM08605, and Jesthom were selected for binding studies. The following MHC allele and reference peptide combinations were used: DRB1*0101 and influenzae peptide HA307-319, PKYVKQNTLKLAT (SEQ ID NO:48) (Busch, R. 1991); DRB1*0701 and Epstein-Barr virus peptide EBV280-290, TVFYNIPPMPL (SEQ ID NO:49) (Omiya et al., 2002); DRB1*1501 and myelin basic protein MBP84-100, ENPWHFFANIVTPRTP (SEQ ID NO:50) (Fridkis-Hareli et al., 2001). A biotin molecule followed by a caproic acid-caproic acid spacer was added to the N-terminus of the reference peptides. The binding of biotinylated peptides to the MHC molecules was tested at various pH values. The pH 5.5 was used to test competition with IFN-β peptides. Detection of peptide binding was done with streptavidin-AlexaFluor546 (Molecular Probes, Eugene, O.R.) in a PCA-96 flow cell counter (Guava Technologies Inc., Hayward, Calif.). Antibodies against DR (Catalog number P01131 M, Biodesign International, Saco Me.), DQ (Catalog number IM0416; Beckman Coulter, Fullerton, Calif.), and DP (Catalog number 552942; BD Biosciences) were used for blocking studies.

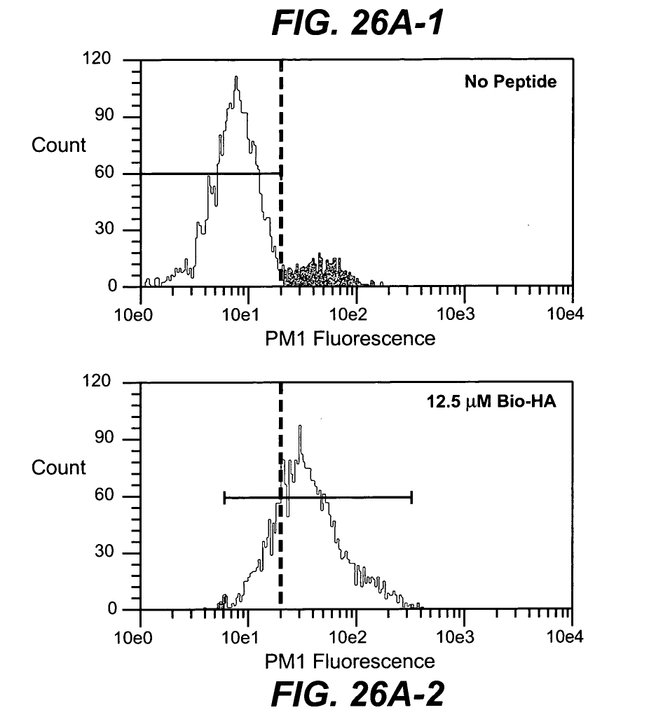

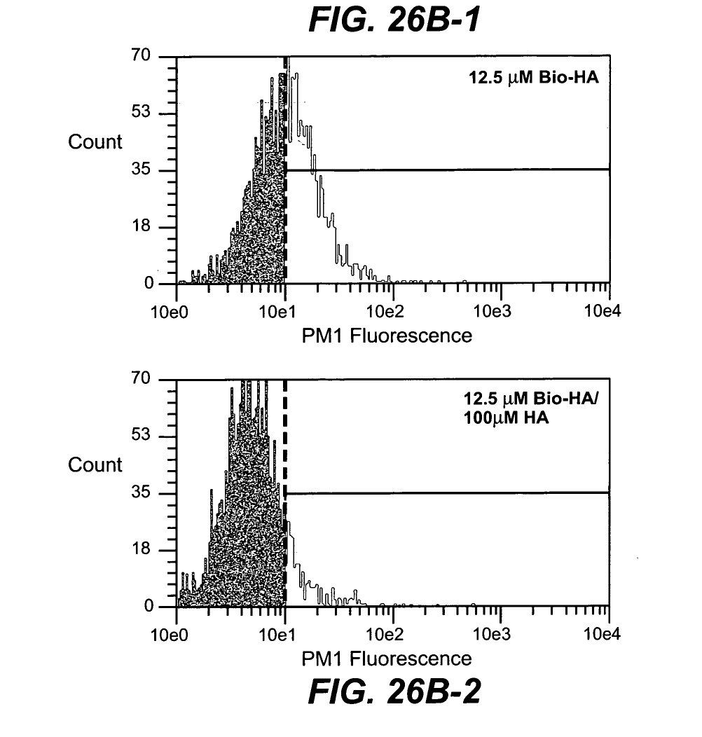

FIG. 26 shows development of a binding assay using B-cell lines with the HLA-II type DRB1*0101 and influenzae peptide HA307-319 with or without biotin label (Bio-HA or HA, respectively). (A) Binding was allowed to proceed for 2.5 hours; (B) Overnight binding. Unlabeled cells are shown at the left of the vertical dotted line, and biotin-peptide labeled cells at the right of the vertical dotted line.

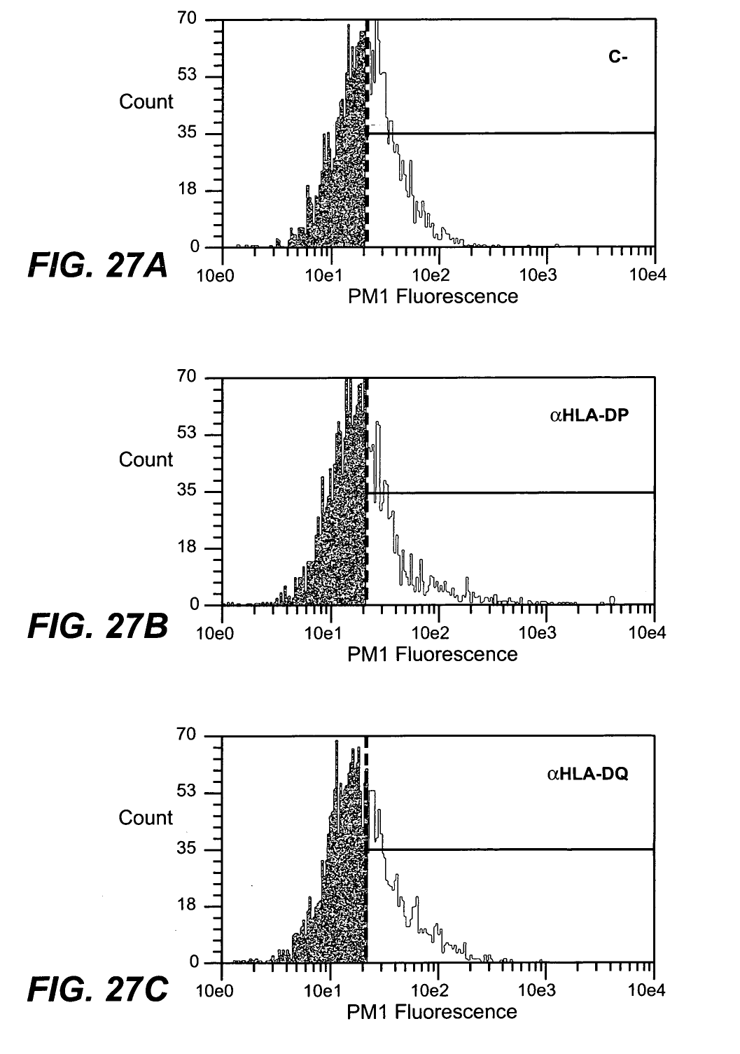

FIG. 27 shows Bio-HA peptide binding to DRB1*0101 in the presence of anti DQ and anti DP antibodies. The cells were labeled with 12.5 μM Bio-HA peptide, in the absence (C−) or presence of blocking antibodies. Unlabeled cells are shown at the left of the vertical dotted line, and biotin-peptide labeled cells at the right of the vertical dotted line.

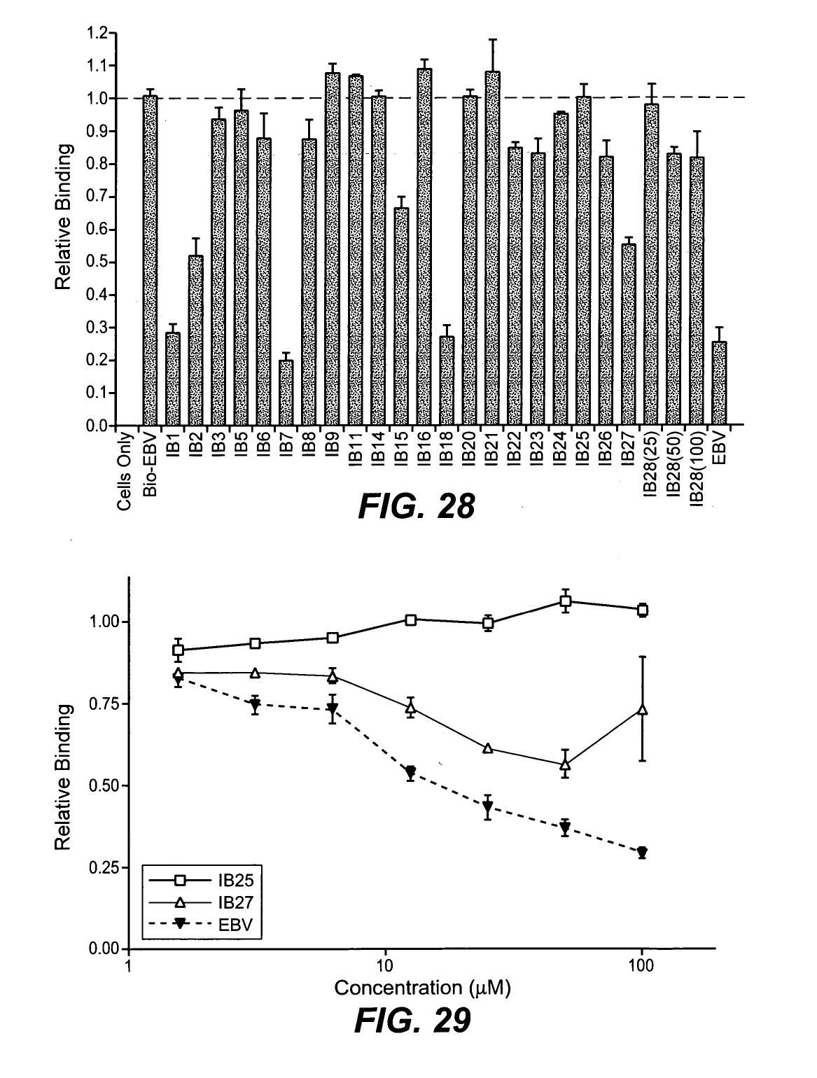

FIG. 28 shows competition of EBV280-290 peptide binding to B cells (DRB1*0701 DQA1*0201 DQB1*0202) by interferon beta peptides. The concentration of Bio-EBV was fixed at 3 μM and the interferon beta peptides tested at 100 μM. Binding was for 2.5 hours.

FIG. 29 shows dose-dependent competition of EBV280-290 peptide binding to B cells (DRB1*0701 DQA1*0201 DQB1*0202) with the interferon beta peptide HYLKAKEYSHCAWTIVR.

All references cited herein are incorporated by reference in their entirety. Whereas particular embodiments of the invention have been described herein for purposes of illustration, it is appreciated by those skilled in the art that numerous variations of the details may be made without departing from the invention as described in the appended claims.

What is claimed is:

1. A method of determining the immunological compatibility of a subject with a therapeutic agent comprising:
determining at least one MHC allele expressed by the subject; and
comparing the at least one MHC allele expressed by the subject with an immunological correlation between said MHC allele and the immunogenicity of the therapeutic agent to determine the immunological compatibility of the subject with the therapeutic agent.

2. The method of claim 1, wherein the subject is human.

3. The method of claim 1, wherein the therapeutic agent comprises a protein.

4. The method of claim 3, wherein the protein is interferon-R.

5. A method of selecting a therapeutic agent for a subject in need of treatment comprising:
determining the immunological compatibility of the subject with the therapeutic agent according to claim 1;
treating the patient with the therapeutic agent if the subject is immunologically compatible with the therapeutic agent.

6. The method of claim 5, wherein the subject is human.

7. The method of claim 5, wherein the therapeutic agent comprises a protein.

8. The method of claim 7, wherein the protein is interferon-R.

9. A method of designing a therapeutic agent with reduced immunogenicity for a subject comprising:
determining the immunological compatibility of a subject and a therapeutic agent according to the method of claim 1; and
designing a derivative of the therapeutic agent, said derivative having reduced immunogenicity in the subject.

10. The method of claim 9, wherein the subject is human.

11. The method of claim 9, wherein the therapeutic agent comprises a protein.

12. The method of claim 11, wherein the protein is interferon-R.

13. The method of claim 11, wherein at least a portion of said derivative has a decreased binding affinity to the MHC allele.

14. A method of designing a vaccine with enhanced immunogenicity for a subject comprising:
determining the immunological compatibility of a subject and a therapeutic agent according to the method of claim 1; and
designing a derivative of the therapeutic agent, said derivative having enhanced immunogenicity in the subject.

15. A method of correlating at least one MHC allele with the immunogenicity of a therapeutic agent, comprising:
determining at least some of the MHC alleles expressed by each of a plurality of subjects;
administering the therapeutic agent to the plurality of subjects;
measuring the immunogenic response of each of the plurality of subjects to the therapeutic agent;
comparing at least one MHC allele in at least one of said subjects with the immunogenic response of the plurality of subjects expressing said allele to correlate at least one allele with the immunogenicity of a therapeutic agent.

16. The method of claim 15, wherein the subject is human.

17. The method of claim 15, wherein the therapeutic agent comprises a protein.

18. The method of claim 17, wherein the protein is interferon-β.
